(19) United States
(12) Patent Application Publication
(10) Pub. No.: US 2021/0230541 A1
(43) Pub. Date: Jul. 29, 2021

(54) Sexed Sperm Bulk Separation Systems

(71) Applicant: Cytolutions, LLC, Columbus, OH (US)

(72) Inventor: Kristie Krug, Upper Arlington, OH (US)

(73) Assignee: Cytolutions, LLC, Columbus, OH (US)

(21) Appl. No.: 16/771,191

(22) PCT Filed: Nov. 9, 2018
(86) PCT No.: PCT/US2018/060178

§ 371 Date: Jun. 9, 2020

Related U.S. Application Data
(60) Provisional application No. 62/584,598, filed on Nov. 10, 2017.

(51) Int. Cl. C12N 5/071; C12N 13/00

(52) U.S. Cl. CPC C12N 5/0612; A61D 19/02; C12N 13/00

(57) ABSTRACT

A process to bulk sex-select sperm which can involve such steps as: obtaining a subject sperm sample such as a collection of cells (1); inducing a sex-based differential alteration process for sperm in the sperm sample; presenting associationally active elements near the sperm within at least some of the sperm sample perhaps such as a fluid combination (4); causing such elements to differentially associate with at least portions of the elements based upon a sperm sex-based differential alteration state; acting on the elements together with their associated sperm through a separation modality (5) to bulk separate the sperm according to their differential sex-based properties. One type of associationally active element is potentially magnetic particles or differentially associatable particles (3) which may even be magnetic particles with inherent zeta potential charges that may associationally act and perhaps bind to an opposite zeta potential or other charges that the sperm may differentially acquire or achieve. This may present a method for magnetic separation of X-bearing and Y-bearing sperm perhaps such as having different charged membranes and perhaps such as after sialic acid activation.

### TECHNICAL FIELD

This invention relates generally to the field of sex selection of sperm such as is useful in producing offspring of a desired gender. It relates to the selection of sperm using sex-based characteristics and can even apply beyond such applications. In some specifics, it involves processes whereby sex selection of sperm can occur through bulk processing of sperm. In one embodiment it can involve the use of magnetic modalities for cellular identification and separation such as for sex selection of sperm.

### BACKGROUND

The selection of sperm based on sex-related characteristics is an area that has become well developed through a particular technology. It is a field that developed largely as a result of the invention disclosed in 1992 in U.S. Pat. No. 5,135,759 to Johnson, et al. from work at the US Department of Agriculture. The Johnson patent explained the ability to utilize a particular DNA staining dye, Hoechst 33342, to individually discern DNA quantity and therefore exploit that fact that X and Y chromosome-bearing sperm have differing DNA content. As that patent noted, the differences were 3.4% in boar, 3.8% in bull, and 4.2% in ram sperm, and these differences could be detected by a flow cytometer with the individual cells then separated by the flow cytometer to yield X and Y sperm sample with over 80% purity. This individual cell detection-based process has been improved over the years but it still remains the only practically usable way in which such results are achieved. The sex selection of sperm is mostly done using a fluorescent dye that labels the DNA content and thus disparity of the sperm and individual cells are then subsequently sorted into X and Y populations of sperm using flow-based cytometry or the like. Although some such processes can yield greater than 90% purity in each sorted fraction, individual cell-based processes such as the flow cytometry sorting of sperm is inefficient and time consuming. It can also result in greatly damaged sperm due to sheer stresses inherent in a flow cytometer, the isolation of the individual cells for individual analysis, possibly the staining of the cells, and the like. These effects can be significant especially for sperm cells because they can be considered from some perspectives as more fragile cells where the desired functioning (indeed, fertilization is the desired function for a sperm cell) can be adversely impacted by large variety of conditions, treatments, or environments. In this regard, the present invention offers advantages that can be especially noticed in maintaining the viability and suitability for their desired purpose when used in practical and real-world processes that can exist such as in an agricultural and ranching setting as is often the case.

Interestingly, a bulk sex selection separation process that can occur in a matter of minutes, separate larger numbers of cells, and is more gentle on the cellular structure has been desired for years. However, this has not been able to be practically, or perhaps repeatedly, achieved even when known and studied processes and techniques were applied. For example, over twenty years ago, it was suggested by Parrish et al. that a bulk separation of bovine sperm from plasma could be achieved by either swim-up or Percoll gradient separation methods. Theriogenology 1995; 44:859-69. Expansion of this technique and application to regular sperm processing did not occur however. In fact, studies, such as a study by Roelf in 1992, and the fact that years on no techniques other than flow cytometry exist are stark testimony to the fact that such proposals were never enabled. And, at this point in time, it is simply true that no practical and repeatable alternative ways of achieving sex-based separation other than flow cytometry exist. The long-desired bulk sex-based characteristic separation for sperm has just continued to remained unavailable.

For example, in 2009 Machado et al. proposed a simple use of the DNA differences as a reason for differences in weight and density as a grounds for the desired bulk separation. Theriogenology 71:1289-97, 2009. However, while it was postulated that the known difference in DNA mass led to a difference in weight and density between X and Y chromosome bearing sperm, this may not have been correct because it did not result in a repeatable or practical bulk sex selection separation process. In fact, one of the issues that may have led to faulty conclusion may have been the type of tests used to determine if there were, indeed, different sex chromosome bearing sperm in the result. At one time, testing for an F-body was deemed indicative of the sex chromosome of the particular sperm cells. As references subsequently explained, this was either not correct or did not yield repeatable results and so often times what was believed to be an enabling disclosure of a repeatable, actually existing effect was not proven to be true either over time, by subsequent implementation for what the alleged process promised, or through subsequent attempts at verification and/or testing of the alleged procedure.

Whether applied to a discontinuous or continuous gradient, or whether used with application of Percoll density gradient centrifugation, the ability to use these technologies did not result in skewing the outcome of the X or Y sperm. Even though motility was increased, the ratios of X bearing and Y bearing sperm remained as a 1:1 ratio. Thus, in even an attempt to apply the Percoll gradient separation technique over a decade after it was previously considered, there was no actionable differential. Thus, at that time, it was concluded that Percoll use for the separation of sperm X bearing chromosomes from Y bearing chromosomes based on weight and density could not be used as it did not alter the 1:1 ratio of X to Y cells from an ejaculate.

Even the application of other bulk processing technologies to sperm themselves have not led to a bulk sex characteristic separation process. For example, the use of electrical charge, zeta charge, and even magnetic particles and/or molecules for certain biological processes or application has been known for at least some years. As disclosed in US Patent Publication US2012/0270204 to Fox et al. in 2012 the magnetic particle technique was even applied to achieve a pre-cytometer sperm sorting treatment to remove dead sperm prior to using a flow cytometer for sex selection. As this reference taught, the expectation was not to use that process for bulk sex selection, but rather to use it as a pretreatment eliminate dead or dying sperm so that the age-old flow cytometer individual cell separation process could be more efficiently achieved. Similar pretreatment was the anticipated goal and all that was indeed achieved by subsequent efforts such as those by the present inventor in US Patent Publication US2014/0234864 to Krug as there was then no known way to improve such processes to achieve sex selection of sperm. Even the general application of electric charge characteristics by Chan et al. over a decade ago in 2006 to evaluate and remove all sperm (i.e., regardless of sex characteristic) having poor DNA integrity did not provide those skilled in the art any ability to achieve a bulk sex separation process as even the use involving sperm DNA itself did not lead to any ability to use that or even the known difference in total DNA content as some type of bulk sex selection modality.

### DISCLOSURE OF INVENTION

Accordingly, the present invention provides a method whereby sperm can be practically and repeatedly separated in bulk, based on a sex characteristic. The overall invention of a bulk sex selection process is presented in a manner that includes a variety of aspects or embodiments which may be applied in different manners with differing values or attributes and in differing combinations to suit the needs of the user and as may be optimal for any type of application. At one level the invention presents a process whereby sperm can be induced to exhibit an attribute that differs between X and Y bearing sperm. This attribute can be used perhaps at the optimal time or in an optimal manner to allow one type of sperm to be preferentially selected and then separated from the other.

One general objective, of embodiments of the present invention may be to present a method whereby sperm can be bulk separated based on their sex related characteristics. Another object of the invention may be to provide a method that may allow faster separation processes as compared to the speed with which individual cell identification-based processes are achieved.

Another object of the invention may be to provide technologies whereby sperm may not be subjected to harsh or damaging environments so sperm separated can be more viable and perhaps more useful for their desired processes such as insemination or the like.

Yet another object of the invention may be to provide a sex selection process that has the ability to quickly and reliably achieve higher separation purity than existing processes.

Still another object of the invention may be to provide sex selection processes that may be less expensive and more easily achieved with less reliance on needs for expensive or complex equipment or highly trained equipment operators.

Through embodiments of the present invention, it may now be possible to induce a sex-based differential change and to use this change to bulk separate sperm based on sex-related characteristics. One embodiment of the present invention may provide a method for separating X bearing sperm from Y bearing sperm in an ejaculate. Benefits and advantages of the present invention include, but are not limited to, bulk purification of one sperm population from another in a rapid, gentle process. This process can result in a higher sperm number separated in a minimal amount of time with less cellular damage. In some embodiments, sperm can be sex selected magnetically based on a difference perhaps such as surface charge and perhaps such as during the capacitation process prior to fertilization.

As but one example of a type of process to get a bulk sex-separated result, sperm can be diluted, pH altered, incubated in a manner to induce a differential change such as capacitation, perhaps mixed with a bulk material or substance that can act on a differential change, and then separated in bulk based properties of either the cells themselves or of the materials or substance that may be mixed with those cells.

As a specific initial embodiment to facilitate capacitation, the pH and incubation time of the sperm may be altered. Sperm that is diluted perhaps such as in TRIS buffers or left to sit at room temperature can maintain a pH range of 6.5 to 6.8 for a longer period of time. A capacitation media can be applied to induce capacitation or changes that may be associated with capacitation.

Of course, as those skilled in the art would readily appreciate, different media can have different properties and one can potentially have more benefit than another for certain applications or certain cell species. Once capacitated, sperm may alter a property perhaps such as causing a net negative charge or neutral charge to become a slightly positive charge. Again, the cutoff time period and most effective pH or other environment to cause such a change or even to capacitate the sperm can be varied. As one example, capacitation time cutoff may be based on the number of X sperm remaining that still have good motility and viability.

Interestingly, in this embodiment, the way in which the sperm are handled or capacitation or other changes are induced can allow or cause the Y chromosome bearing sperm to achieve a change or an amount of change faster or to a greater degree than X chromosome bearing sperm and this difference can be exploited to achieve the desired bulk or other different type of separation. Importantly, these changes can be induced while leaving a number of viable X sperm available to process after removal of Y sperm or the like. In some embodiments, such changes can cause or use a change in surface charge of the capacitated sperm as a differential effect for separation.

To achieve the foregoing, and in accordance with some purposes of embodiments of the present invention as broadly described herein, a method for separating X bearing sperm from Y bearing sperm may include but is not limited to: using magnetic particles with inherent zeta potential charges to bind to different subpopulations of sperm, and separating the cellular or otherwise bound magnetic particles such as in the presence of a magnetic field, a negatively charged substrate such as glass or silica, dextran, or any other substrate or effect to which the negative zeta charge reacts such as in the presence of buffers or otherwise. With reference to sperm, embodiments of the invention can include, a method for separating X bearing sperm from Y bearing sperm, hereof, perhaps including: obtaining charged particles; mixing conjugated or unconjugated, perhaps charged magnetic particles with a sample of sperm; and separating the sperm bound to magnetic particles in the presence of a magnetic field or the like.

Additional objects, advantages and novel features of the invention are set forth in part in the description which follows, and in part will become apparent to those skilled in the art upon examination of the following or may be learned by practice of the invention or adaptation of its basic principles to particular situations, cells, substances, or species. The objects and advantages of the invention may be realized and attained by means of the instrumentalities and combinations particularly pointed out as well as those items shown by inference.

### BRIEF DESCRIPTION OF DRAWINGS

The accompanying figures, which are incorporated in and form a part of the specification, illustrate one or more embodiments of the present invention as related to sperm, and, together with the description, serve to explain the broad general principles of the invention. In the figures:

FIG. 1 illustrates a pH change in neat sperm over time at room temperature in various buffers with various pHs for a first bull.

FIG. 2 illustrates a pH change in neat sperm over time at room temperature in various buffers with various pHs for a second bull.

FIG. 3 illustrates a pH change in neat sperm over time at room temperature in various buffers with various pHs for a third bull.

FIG. 4 illustrates a pH change in neat sperm over time at room temperature in various buffers with various pHs for a fourth bull.

FIG. 5 illustrates an example of the pH value of frozen thawed purified X and Y sperm as well as conventional sperm in an Sp-TALP-H buffer over time.

FIG. 6 illustrates a capacitation possibly as deduced from pH change of Y sperm in increasingly concentrated heparin buffers over time.

FIG. 7 illustrates a flow cytometry histogram of Y bearing sperm for which capacitation was induced by 10 ug/ml heparin for three hours.

FIG. 8 illustrates a zeta potential of iron oxide nanoparticles that were mixed with Sp-TALP-H at the level of 10 ug/ml heparin.

FIG. 9 illustrates a zeta potential of iron oxide nanoparticles that were mixed with Sp-TALP-H at the level of 20 ug/ml heparin.

FIG. 10 illustrates a flow cytometry histogram of Y bearing sperm for which capacitation was induced by 20 ug/ml heparin for six hours prior to mixing with magnetic particles.

FIG. 11 illustrates a flow cytometry histogram of Y bearing sperm for which capacitation was similarly induced by 20 ug/ml heparin for six hours after treatment with 1.2 mg of iron nanoparticles for 20 minutes and after magnetic removal by three minutes on a magnet.

FIGS. 12-14 illustrate systems in schematic that include aspects as may be configured according to embodiments of the invention.

### MODE(S) FOR CARRYING OUT THE INVENTION

As mentioned earlier, the present invention includes a variety of aspects, which may be combined in different ways and may be applied to sex selection of sperm including but not limited to in a bulk manner, or perhaps with magnetic nanoparticles or the like. The following descriptions are provided to list elements and describe some of the embodiments of the present invention.

These elements are listed with initial embodiments and are shown in examples of an embodiment relative to sperm, however it should be understood that they may be combined in any manner and in any number to create additional embodiments. The variously described examples and preferred embodiments should not be construed to limit the present invention to only the explicitly described systems, values, amounts, techniques, and applications. The specific embodiment or embodiments shown are initial examples only that those of ordinary skill in the art will readily understand may be altered and optimized for the particular applications and results desired. The specification should be understood and is intended as supporting broad claims as well as each embodiment, and even claims where other embodiments may be excluded. Importantly, disclosure of merely exemplary embodiments is not meant to limit the breadth of other more encompassing claims that may be made where such may be only one of several methods or embodiments which could be employed in a broader claim or the like. Further, this description should be understood to support and encompass descriptions and claims of all the various embodiments, values, systems, substances, steps, elements, techniques, methods, devices, and applications with any number of the disclosed elements, with each element alone, and also with any and all various permutations and combinations of all elements in this or any subsequent application.

The initial invention is presented in reference to initial embodiments, however, these should be understood as initial embodiments only. The invention is intended to encompass changes as can be developed to allow bulk separation and the like as a more desirable technique for a variety of reasons ranging from cost, to cell impacts, to results achieved. Thus, any of the initially proposed and disclosed steps may be varied and even some eliminated and others added as should be understood. With that understanding, embodiments can involve systems and process steps such as: establishing a collection (1) of cells, perhaps sperm cells (2) such as may have both X-bearing sperm cells and Y-bearing sperm cells; utilizing a cell related differential effect that can be applied or used to distinguish somehow between a desired type of cell and ones that are not desired (or vice versa); acting on that effect; and then generally separating some portion of the cells for later use perhaps through various types of a separation modality (5) and even a particle separation modality. In such processes, the cell related differential effect can be of a variety of types. It may be a type of effect that can be considered as intrinsically actionable or exhibiting, and so can manifest or exhibit its differential character in a way that can be used for some type of separation process. Further, it may be desired that it be an effect that is intrinsic in that it need not be augmented or a matter that requires significant or potentially dangerous outside influence or chemicals (e.g., as in a stain) in order to be utilized or applied in a most commercially acceptable manner. Again, this can serve a purpose desired for some embodiments of minimizing any impact of the cells or their desired functions. Systems can involve differentially exhibiting cells, sex chromosome differentially exhibiting sperm cells, or the like.

Recognizing that a number of authors in the sperm sexing field have announced that they have achieved the long desired holy grail of a bulk sperm sex-based separation process only to have subsequently been proven wrong and to have not enabled what they suggested they did, an aspect of embodiments of the invention can be to provide repeatable, controllable processes for those in the field to practically apply. From this perspective, embodiments of the invention can include processes that can be adapted to cause, or perhaps controlling to allow or assure, that the differential effect exists precisely when desired so it can be repeatably applied. Perhaps at its simplest, this might be through a process of just affirmatively supporting the intrinsically actionable sperm cell sex chromosome related differential effect for the collection of sperm cells, or providing a differential effect affirmative support (10) such as a condition to allow thawing and transitioning in a controlled manner, an appropriate pH increasing buffer, or the like, so that it can be assured to be present when needed. This can be particularly important because the differential effect can be one of a transient nature itself. And the differential effect can come with a need to balance its effects to maintain other needed aspects such as cell viability or function or the like.

The process is desired to be highly repeatable and so embodiments can involve timing or a window of opportunity that may be critical.

Such is especially the case for sperm cells where viability is of paramount importance. Here, the aspect of possibly just allowing the differential effect to exist without significantly compromising use of any of the sperm cells for fertilization processes can be desired. Given prior announcements that were only followed by failure to prove or repeat, an important aspect of affirmatively supporting can be the act of actually supporting the differential effect such as by causing it, by giving it a favorable environment, or perhaps just by providing a transition state timer (14) or timing after a particular known event so that the entire process not only is enabled, but it works both reliably and repeatably. This can be significant for embodiments where the sex differential property of the cells such as the sperm cell sex chromosome related differential effect of sperm cells is transitory, subtly exhibited, or short-lived when considered in the window of maintaining the cells as viable or the like.

As mentioned, an aspect of embodiments of the invention can be that of utilizing the cell related differential effect so that it can be applied or used to distinguish a desired type of cell. In embodiments of the invention, it is possible to utilize the differential effect by using it to create an association such as with some substance (6), a surface, or even a particle. The association may be a differential association, so that perhaps only one type of cells is associated while the other type of cells is not associated. And the modality for associating, be it a substance, surface, particles, or the like can be differentially associatable such as when it/they are capable of establishing or having established for it/them an association with the desired or undesired type of cells as the case may be. The modality for associating, substance, surface, particles, or the like can be used to establish a suspension, mixture, colloid, or any other type of structure in a fluid combination so it can be mobile and can act so as to be proximal to most if not all the cells and this is one-way users can achieve proximally situating a substance in the vicinity of the collection of cells. For example, by suspending or otherwise combining associationally active particles in a buffer they may be easily fluidically combined with the sex chromosome differential exhibiting sperm cells whereby there can be established a fluid combination (4) of sperm cell sex chromosome differentially associatable particles and sex chromosome differentially exhibiting sperm cells. These two types of things can then or later act so that some become associated with the desired or undesired types of cells and thus can then form a differentially associated substance, differentially associated particle, differentially associated surface, or the like. Thus, there may be differentially associatable particles (3), or for the sperm sexing applications, there may be sperm cell sex chromosome differentially associatable particles that associate differently (perhaps under the appropriate conditions or such) based on the very nature of whether or not that particular sperm cell is an X-bearing sperm cell or a Y-bearing sperm cell.

For X-bearing sperm cells and Y-bearing sperm cells it can be significant that only one of the two types of cells are associated with the differentially associatable substance, surface, particles, or the like. This is because the other is essentially unaffected and has none or very little of the actually or potentially negative treatments, environments, or foreign substances that may affect its natural efficacy. Thus, embodiments of the invention can involve associating only a desired portion of the collection of sperm cells with the substance, surface, particles, or the like. As mentioned, the invention can generally involve separating some portion of the cells for later use perhaps through some type of separation modality (5), or even more specifically, a particle separation modality. Separating (as is understood from the word) involves removing one from the other. Of course, it does not matter which is removed from which within the meaning of separating; one type can be simply removed from another type. Note, however, from the perspective of viability, especially of sperm cells, cells that are associated or perhaps even bound to the separation modality such as a particle may not be in a usable form and may not ultimately be considered viable even though they may have started out that way. As a result, systems can be designed to achieve capture of one or the other types of cells where possible. And it may be that the desired type of cell and ones that are not desired are reversed in some instances.

The separation can occur from some separation modality (5) and can also be based upon a differential effect, perhaps such as an intrinsically actionable sperm cell sex chromosome related differential effect for some embodiments. For embodiments involving a use of particles, the particles can be considered one part of the separation modality. Another part of the particle separation modality can be some external actor, perhaps including but not limited to a force or application of a force to which the sperm cell sex chromosome differentially associatable particles in the fluid combination of sperm cell sex chromosome differentially associatable particles and sex chromosome differentially exhibiting sperm cells are responsive. As explained elsewhere, where there is a force, the force can be a magnetic force, centrifugal force, or perhaps just gravity or a gravitational force. It could also be an electrostatic force or even forces such as are encountered in an electrophoresis process. Regardless of the components or nature of the separation modality, the process can involve separating at least some of the differing types of cells, perhaps such as the X-bearing sperm cells and Y-bearing sperm cells.

In practice, some embodiments of the invention can involve some combination such as the following steps: 1) obtaining a sample; 2) incubating the sample in an appropriate buffer to induce a desired differential effect; 3) suspending or otherwise combining associationally active particles in a similar or perhaps identical buffer; 4) mixing an aliquot of suspended particles with suspended cells at desired ratios; 5) incubating the mixture for a desired period of time; 6) subjecting the mixture to a separation modality for a set period of time; and 7) separating, perhaps by removing, a portion of the mixture so as to yield a desired resultant selected portion of the total. Further, optional steps could include, among other options including but not limited to: alteration of the aforementioned steps; washing the sample with buffer before incubating to induce the desired effect; resuspending the sample in the same buffer after incubation or perhaps in the same buffer with the addition of bovine serum albumin (BSA) of a similar substance; achieving a desired final cell concentration such as perhaps about 160 M cells/ml; coordinating a particle amount with the cell amount in some measurable manner; using an unfrozen sample or perhaps thawing a frozen semen amount to obtain the sample prior to achieving separation on the thawed sample; incubating for differing time periods; and the like.

The present invention may include a method for separating X bearing sperm from Y bearing sperm through a bulk process or in otherwise new manners thereby enriching a sperm subpopulation. Briefly, an initial embodiment of the present invention can include a method for separating X bearing sperm from Y bearing sperm using magnetic selection as but one example.

The method can be applied to cells such as contained in freshly collected samples after dilution, during and after cooling, or during and after other cell or system procedures that are employed prior to cryopreservation, or to frozen/thawed cell samples and the like. The enriched cell populations can be used for routine procedures, prior to or after other processing techniques, prior to or after shipment of samples, and prior to or after cryopreservation or other processes. These separated samples can be used for in vitro fertilization, for all mammalian sperm or the like.

In one general manner, the invention can be understood as if exploiting the aspects of capacitation and zeta charge changes associated therewith in a new manner that allows or perhaps induces such changes differentially for the X- versus the Y-bearing sperm cells. The existence of a differentiation can arise such as by affirmatively supporting an intrinsically actionable effect for any type of cell, perhaps such as a sperm cell sex chromosome related differential effect for a collection of sperm cells. In keeping with the above, the differential effect can be a naturally occurring cellular process. If well predictable, this naturally occurring cellular process can occur spontaneously, but it may be most controllable if it is triggered or controlled by an outside influence perhaps such as an environmental condition, perhaps such as by affirmatively supporting a cellular process differential transition effect or providing a cellular process differential effect affirmative support, it may be caused such as by a separately supplied, perhaps well-accepted chemical influence that does not significantly impact the cells. As mentioned above, the naturally occurring cellular process can be a process that has a transient character in that it can involve a relatively short-term changing state. It can simply be a transition effect whereby cells may be transitioning from one state to another. An example of such a process for sperm cells is that of the capacitation process. Of course, capacitation, a process whereby sperm cells become capable of fertilizing, is a naturally occurring process. As can be understood from the natural act of fertilization, it can be triggered or effected through environmental conditions and/or chemical inducements and both can be used when fertilization is desired. Thus, the differential effect, perhaps such as a capacitation effect, can occur as a result of triggering capacitation for a collection of sperm cells or a capacitation trigger (13).

As background, it can be understood that once capacitated, sperm can be understood as altering the net negative charge found inherently, to a neutral or slightly positive net charge. The invention can exploit this in a manner that determines a cutoff time period or the like for such a change. This can be determined as perhaps the most effective pH change to capacitate Y chromosome bearing sperm faster or in a larger amount or higher total amount than X chromosome bearing sperm. In one example, this can be done while leaving a number of viable X sperm available to process after removal of Y sperm using an effect perhaps such as the change in surface charge of the capacitated sperm to be used for separation.

In keeping with the aspect of having a controlled, predictable, and repeatable process that is not dependent on just lucky timing which can be unrepeatable, a number of differential effects can be considered as the principles of the invention are applied to various cells, various systems, and various desires. A surface area effect can be involved and actions can be taken such as might predictably cause triggering of a differential surface area effect for a collection of cells, perhaps such as sperm cells and can be provided by a differential surface area effect trigger.

There can also be a charge effect, a surface charge effect, and even a zeta charge effect. And again, elements can be provided such as a differential charge effect trigger or actions can be taken that might trigger a differential charge effect for a collection of cells, perhaps such as sperm cells, trigger a differential surface charge effect for a collection of cells, or even trigger a differential zeta charge effect for a collection of cells. It is also possible to induce an effect such as by not just waiting for it to occur but by very causally affirmatively acting to make the effect occur. There can thus be a differential effect inducer (12), a sperm cell chemistry differential effect inducer, a sperm cell carbohydrate differential effect inducer, a sperm cell sialic group differential effect inducer, a polymerase based inducer, a receptor molecule inducer, a Cas9-type inducer, a CRISPR-type inducer, a DNA tag inducer, or processes such as inducing a sperm cell charge differential effect, inducing a sperm cell surface charge differential effect, generally differential effect inducing (of any types of cells for later separation), sperm cell chemistry differential effect inducing, sperm cell carbohydrate differential effect inducing, sperm cell sialic group differential effect inducing, polymerase based inducing, receptor molecule inducing, Cas9-type inducing, CRISPR-type inducing, DNA tag inducing, and even inducing a sperm cell zeta charge differential effect or providing a sperm cell zeta charge differential inducer. For embodiments where the cells exist in a fluid medium, the zeta charge changes whereby a differential is exhibited can include movement to a substantially uncharged state or perhaps just movement to a low charged state. Thus, embodiments of the invention can involve inducing a sperm cell substantially uncharged differential effect and inducing a sperm cell low charge differential effect where appropriate or provide a sperm cell substantially uncharged differential inducer or a sperm cell low charge differential inducer. Other potentially usable and designable differential effects can include: sialic changes, silane surface values, a cell sialic group effect, a cell surface cleaving effect, a cell sialic group cleaving effect, a cell chemistry effect, a cell electrical value, a cell electrostatic effect, a cell carbohydrate effect, a cell surface substance existence, a cell surface property, a cell pH value, a cell ion value, a cell membrane effect and the like. Here, systems can involve steps such as inducing a sperm cell chemistry differential effect, inducing a sperm cell carbohydrate differential effect, inducing a sperm cell sialic group differential effect, inducing a sperm cell sialic group cleaving differential effect, or the like. So, again as but one example, systems can be designed to include a sialic acid group differentially associated substance, differentially associating with cells such as sperm cells in a collection of cells such as sperm cells based upon sialic acid group content, and separating at least some of said cells, perhaps such as X-bearing sperm and Y-bearing sperm cells, based on their sialic acid group content or the like.

In some embodiments, an increase of the pH by at least about 0.36 units can be exploited as an indication when sperm start to capacitate and/or die and this can be achieved in a manner to effect a usable change such as the charge properties of the cell, such as a zeta charge effect. To achieve these changes, a media in which the sperm are stored can contribute to this increase in pH, so can a passage of time. In example 1, a test of four bull ejaculates in four conditions were analyzed over a period of six hours as explained below.

Understanding some underlying processes involved in some embodiments and how these can be varied can contribute to an understanding of the breadth of the invention described in its initial embodiments. In the basic process for sperm cells, it can be understood that as sperm capacitate, there is a loss of sialic acid groups on the surface of the cell. Potentially involved in the differential processes for X and Y sperm as explained here, it can be appreciated that there may be a disproportionate amount of sialic acid on Y sperm as opposed to X sperm, perhaps whereas X sperm have a higher amount of sialic acid groups. Living sperm contained in certain buffers as well as in the reproductive tract can be understood as having a net negative zeta potential. Perhaps as sialic acid groups are lost such as during capacitation (the penultimate step prior to fertilization) the net zeta potential charge of the sperm may reach zero to slightly positive mV. To the degree that X sperm contain more sialic acid groups than Y sperm, the process to reach a zero to slightly positive zeta potential charge can take longer or be achieved to a more significant degree than Y sperm. Therefore, a careful titration to cleave sialic acid groups and the subsequent addition of negatively charged substrate may yield a purified X bearing sperm population.

An indication of such changes or others that can be exploited can be in the pH of the media suspending the cells. This can affect the charge of proteins comprising the cells. Proteins function as dipolar ions mainly due to the ionization of the various R groups of the amino acids, which make up their primary structure. Thus, media pH may affect their protein-protein interactions. In the example of sperm, capacitation can involve the removal of seminal coating proteins absorbed on the sperm's surface membrane, thus, changing the pH of the capacitating medium from pH 7.2 through pH 8.4 could be expected to alter the binding of these proteins to the sperm's surface and aid in effecting the desired change.

Ionic components of the culture medium can influence mammalian sperm motility, capacitation, acrosomal integrity, and a sperm's ability to penetrate an oocyte. The pH of the medium can affect the ionization of substances within it, including proteins intrinsic to the sperm membrane and extrinsic, absorbed seminal plasma proteins. The pH of the medium can also determine many important aspects of the structure and function of biological macromolecules, including enzyme activity, and thus can act to determine the behavior of cells. Using these aspects, embodiments of the invention can apply processes and substances in a new way to differentially impact characteristics such as the net charge on the surface of the cell. This can be affected by the pH of the cell's surrounding environment. And the cell can become more positively or negatively charged due to the loss or gain of protons. At or near physiological sperm pH, the net surface charge can be considered as being negative. Further, biological membranes, including sperm, are considered negatively charged in physiological pH. This may be due mainly as a result of the presence of acidic phospholipids; about 10-20% of the total membrane lipids are anionic ones.

Because the membrane can be exposed to a surrounding aqueous buffer, specific interactions with outer medium components can occur or can be induced by desired processes. A resulting equilibria, in which charged groups of membrane components and solutions ions can be involved, can be affected by different factors and processes leading to a membrane surface charge density variation. The parameter can also be influenced by membrane composition, ionic strength of electrolytes, and solution pH. With this background, it can be understood that viable mammalian sperm can be considered to have a net negative surface charge bound to their plasma membrane. As sperm undergo capacitation followed by the acrosome reaction, it can be considered that their net charge can be reduced and positively charged components can be considered as appearing. Capacitation can thus be considered as characterized by the removal of coating materials from the sperm surface and as the penultimate step in either fertilization resulting in an increased permeability of the plasma membrane to Ca⁺⁺ ions, which allows the sperm to undergo the acrosome reaction or death if fertilization does not occur. Sperm cell's net negative charge increase by capacitation can be demonstrated by selecting sperm based on the negative zeta electrokinetic potential sperm acquire in a specific medium (Chan et al., “A simple zeta method for sperm selection based on membrane charge”). For purposes of understanding how to specifically adapt embodiments of this invention it can be understood that a viable mature human sperm can be understood as having a negative zeta potential of −16 to −20 mV (differential potential between the sperm membrane and its surroundings). Further, this can be understood as decreasing (less negative) upon capacitation and can be understood as becoming more positively charged or at least near zero by capacitation. As further background from which embodiments of the invention can be fine-tuned for specific applications, it should be understood that mammalian sperm are not immediately capable of fertilizing oocytes, rather they must undergo a period of preparation that normally occurs in the female reproductive tract. The changes that occur in sperm can be considered as involving at least two components: an initial sperm membrane alteration that allows the sperm to undergo the second phase, and the fusion of the plasma membrane and outer acrosomal membrane. The first phase can be considered to be the period of capacitation, and the second phase can be considered as the acrosome reaction.

Heparin is a substance that can induce capacitation in sperm leading to fertilization of oocytes. It can be used in embodiments of the invention as one of the ways to achieve inducing a sperm cell sex chromosome related differential effect, to induce capacitation and likely effect a change in the zeta potential of the sperm cells. Surprisingly, this can be a differential effect in X as opposed to Y sperm and can serve as a method to discriminate between the two types of sperm cells. Further, to additionally control the capacitation of the sperm, the pH of the surrounding media can be important. In embodiments of the invention, it can be helpful to introduce the sperm cells to a medium so that the internal pH of the sperm increases from the pH of its baseline ejaculate by some amount, perhaps such as 0.36 pH units. There can also be a time dependence factor where passage of time can cause such desired changes as well. As helpful background to aid in both understanding the invention as well as facilitating available adaptation to specific applications, it may be understood that likely the surface of the sperm during capacitation can lose sialic acid groups as they are incorporated into the sperm membrane. The loss of sialic acid may be responsible for the loss of the net negative charge of live sperm. Further, it appears that human X and Y sperm differ in sialic acid content—Y having less sialic acid than X, and therefore this patent disclosure sets out to demonstrate that sperm bearing X chromosomes can be separated from Y bearing chromosomes based on controlled capacitation of an ejaculate by raising the pH of the ejaculate over time with buffers including heparin, rendering the zeta potential charge of the capacitated sperm neutral to slightly positive and separating the neutral/positive charged sperm with a negatively charged surface or particles yielding a high percentage of X bearing chromosome sperm. Noteworthy is that is it likely that the heparin induced capacitation can cause sialic acid differences in a time period, perhaps such as a six-hour window. The sialic acid differences can contribute to sex selection perhaps based on the differences of sialic acid content and subsequent pH and zeta potential differences of the sperm cells.

As mentioned above, one can control to allow or assure that the differential effect exists when it is desired. A differential effect, such as in but one example, an intrinsically actionable sperm cell sex chromosome related differential effect can be affirmatively supported so that it can be assured to be present when needed. While it may be applied where it exists in other cells, in sperm cells, simply having a stress even can trigger, allow, or induce capacitation and a differential effect. This is true when it is the process of capacitation that is used to cause the differential effect. Stress, such as changes in environment, freezing, thawing, and others can induce the onset of the capacitation processes. Some embodiments can involve affirmatively establishing the collection of sperm cells in a frozen then thawed state. Once in this state, the process can be timed or otherwise measured to get the desired differential effect. To make the process affirmative and thus both controllable and reliably repeatable and usable such as when following the process of allowing the differential effect to occur, the change can be accomplished and timed or otherwise measured to determine if and when an actionable, appropriate, and perhaps balanced (such as with viability, cell function non-impairment, or the like) differential effect exists for use for ultimately a separation process. In this regard, embodiments of the invention can involve affirmatively establishing a collection of cells, perhaps such as sperm cells, in a timed transition state. The change in environment that is used to cause the desired differential effect can include an appropriate change of buffer; although this can potentially also be considered a chemical change. Embodiments can involve subjecting the collection of sperm cells to a differential change inducing buffer. This differential change inducing buffer can be accomplished through the step of subjecting the collection of sperm cells to a buffer containing an operative amount of a salt, or the step of subjecting the collection of sperm cells to a more basic buffer. Naturally for other cells and even for sperm cells, other types of inducements can be designed within the scope of the invention.

While the differential effect can be allowed to exist in a passive manner without significantly compromising use of any of the cells, it can also be so achieved through the process of inducing the differential effect with an inducer, perhaps such as inducing a sperm cell sex chromosome related differential effect. This step of inducing can cause the differential effect to controllably exist whenever it is desired. The step of timing for the desired effect can achieve a step of controlled difference inducing the effect perhaps such as the sperm cell sex chromosome related differential effect or a differential effect difference control. Inducing the desired effect can be achieved chemically and embodiments of the invention can include a differential effect chemical inducer agent or the step of chemically inducing the sperm cell sex chromosome related differential effect. As mentioned above, one chemical that has been discovered to induce a differential effect in sperm cells based on their sex chromosome so far is heparin. Significantly, it is now disclosed that heparin, a chemical generally considered very safe for fertility control, can be used to safely induce a desired differential effect without significant impairment of the viability of the sperm cells if it is used both properly and controlled appropriately. Not enough or too short a use, and there can be not enough of a differential effect; too much or too long and either the differential can decay or it can go away, and/or the viability of the sperm cells can be unacceptably compromised. Thus, embodiments of the invention can involve subjecting the collection of sperm cells to heparin and even having an appropriate concentration of heparin and timing or otherwise determining the amount of the change that is optimal for the amount of differential effect and other considerations.

In keeping with the above, many alternatives exist for the invention and its ability to be designed for particular applications following its principles. A variety of ranges can be used for all options disclosed as only initial possibilities throughout this patent. For uses of heparin as a differential effect inducement, subjecting the collection of sperm cells to heparin can include values perhaps including but not limited to: a concentration of about 5 ug heparin per ml of buffer, a concentration of about 10 ug heparin per ml of buffer, a concentration of about 15 ug heparin per ml of buffer, a concentration of about 20 ug heparin per ml of buffer, a concentration of about 5 ug heparin per ml of buffer for about 120 minutes for sperm cells that have not been previously frozen, a concentration of about 5 ug heparin per ml of buffer for about 180 minutes for sperm cells that have not been previously frozen, a concentration of about 5 ug heparin per ml of buffer for from about 180 minutes to about 240 minutes for sperm cells that have not been previously frozen, a concentration of about 10 ug heparin per ml of buffer for about 120 minutes for sperm cells that have not been previously frozen, a concentration of about 10 ug heparin per ml of buffer for about 180 minutes for sperm cells that have not been previously frozen, a concentration of about 10 ug heparin per ml of buffer for from about 180 minutes to about 240 minutes for sperm cells that have not been previously frozen, a concentration of about 15 ug heparin per ml of buffer for about 120 minutes for sperm cells that have not been previously frozen, a concentration of about 15 ug heparin per ml of buffer for about 180 minutes for sperm cells that have not been previously frozen, a concentration of about 15 ug heparin per ml of buffer for from about 180 minutes to about 240 minutes for sperm cells that have not been previously frozen, a concentration of about 20 ug heparin per ml of buffer for about 120 minutes for sperm cells that have not been previously frozen, a concentration of about 20 ug heparin per ml of buffer for about 180 minutes for sperm cells that have not been previously frozen, a concentration of about 20 ug heparin per ml of buffer for from about 180 minutes to about 240 minutes for sperm cells that have not been previously frozen, a concentration of about 5 ug heparin per ml of buffer for about 30 minutes for thawed previously frozen sperm cells, a concentration of about 5 ug heparin per ml of buffer for about 60 minutes for thawed previously frozen sperm cells, a concentration of about 5 ug heparin per ml of buffer for from about 45 minutes to about 60 minutes for thawed previously frozen sperm cells, a concentration of about 10 ug heparin per ml of buffer for about 30 minutes for thawed previously frozen sperm cells, a concentration of about 10 ug heparin per ml of buffer for about 60 minutes for thawed previously frozen sperm cells, a concentration of about 10 ug heparin per ml of buffer for from about 45 minutes to about 60 minutes for thawed previously frozen sperm cells, a concentration of about 15 ug heparin per ml of buffer for about 30 minutes for thawed previously frozen sperm cells, a concentration of about 15 ug heparin per ml of buffer for about 60 minutes for thawed previously frozen sperm cells, a concentration of about 15 ug heparin per ml of buffer for from about 30 minutes to about 60 minutes for thawed previously frozen sperm cells, a concentration of about 20 ug heparin per ml of buffer for about 30 minutes for thawed previously frozen sperm cells, a concentration of about 20 ug heparin per ml of buffer for about 60 minutes for thawed previously frozen sperm cells, a concentration of about 20 ug heparin per ml of buffer for from about 30 minutes to about 60 minutes for thawed previously frozen sperm cells, until exhibiting an increase of about 0.33 pH, until exhibiting an increase of about 0.36 pH, until exhibiting an increase of about 0.39 pH, until exhibiting an optimal differential effect increase in pH, until exhibiting an optimal cell viability increase in pH, until exhibiting an optimal differential effect increase in pH such as might yield the earliest usable effect, the highest viability, the maximum differential or the like, and until exhibiting an optimal cell viability increase in pH, or any combinations of the above. Caffeine can also serve to induce capacitation and a differential effect. For uses of caffeine as a differential effect inducement, subjecting the collection of sperm cells to caffeine can include designing a system with values perhaps including but not limited to: until exhibiting an increase of about 0.33 pH, until exhibiting an increase of about 0.36 pH, until exhibiting an increase of about 0.39 pH, until exhibiting an optimal differential effect increase in pH such as might yield the earliest usable effect, the highest viability, the maximum differential or the like, and until exhibiting an optimal cell viability increase in pH, or any combinations of the above. Where a pH altering buffer is used to induce capacitation and thus a differential effect, subjecting the collection of sperm cells to a pH altering buffer (15) can include designing a system with values perhaps including but not limited to: subjecting said collection of sperm cells to a buffer having a pH that increases the environment of said collection of sperm cells by about 0.33 pH, subjecting said collection of sperm cells to a buffer having a pH that increases the environment of said collection of sperm cells by about 0.36 pH, subjecting said collection of sperm cells to a buffer having a pH that increases the environment of said collection of sperm cells by about 0.39 pH, or any combinations of the above.

To illustrate the process in its different aspects and to show its likely efficacy, different examples and test events have been conducted. The first example or test is a sequence designed to illustrate the pH change and likely capacitation state variances in neat sperm over time at room temperature in various buffers with various pH's.

### Example 1: Measurement of pH of Fresh Semen in Different Buffers Over Time

This first example is disclosed to show the pH change in neat sperm over time at room temperature in various buffers with various pH's. As background, it should be understood that the ability to facilitate or induce capacitation is useful in some embodiments such as for magnetic removal of capacitated sperm. Naturally it should be understood that these examples are testing initial options only. As those of ordinary skill in the art should well understand, the pH variances and incubation times set for the sperm samples can be altered to suit particular applications and to create other embodiments that still fall within the scope of the present invention.

As part of this initial test, each of these ejaculates were either kept as neat semen (untouched), resuspended in TRIS 300WS buffer, resuspended in a clear TALP buffer, or resuspended in an Sp-TALP-H (heparin 10 ug/ml) buffer as but initial type of buffers that could be used. Because there was a little over 1 ml per ejaculate, 300 ul of ejaculate was pipetted into each of the four groups. The neat ejaculate was not diluted, whereas the other groups had 700 ul of buffer added to each tube.

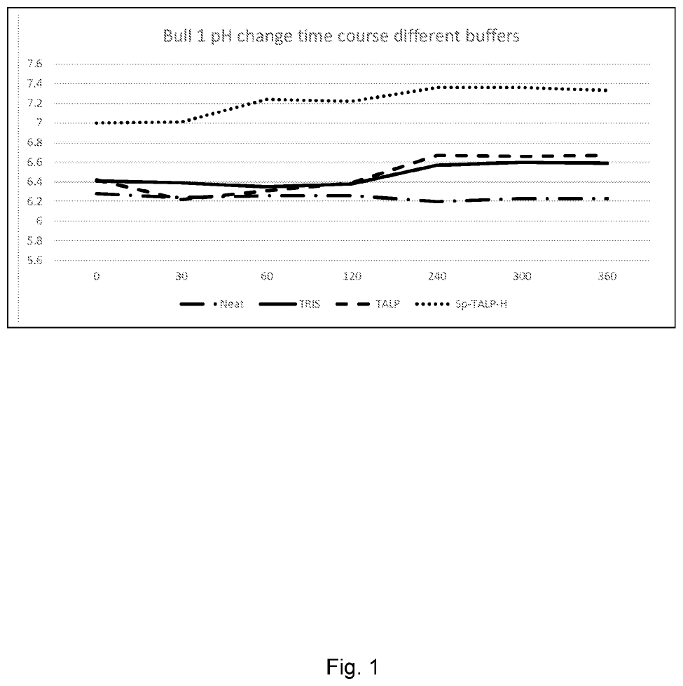

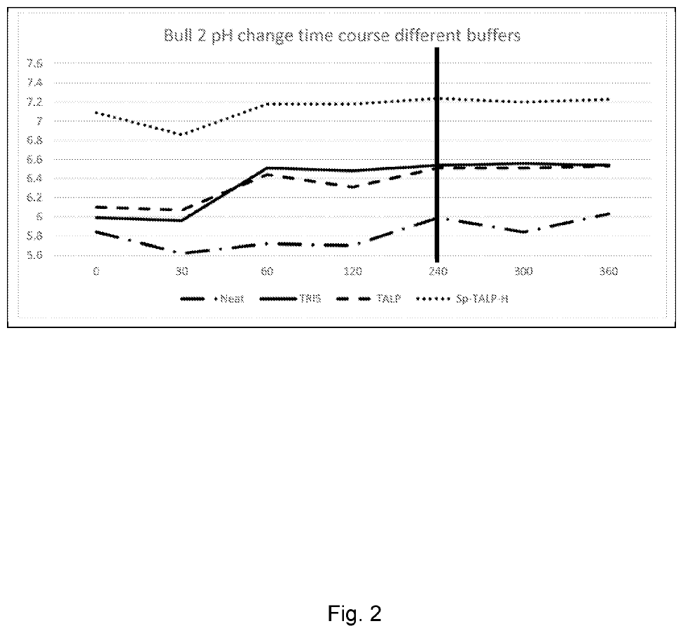

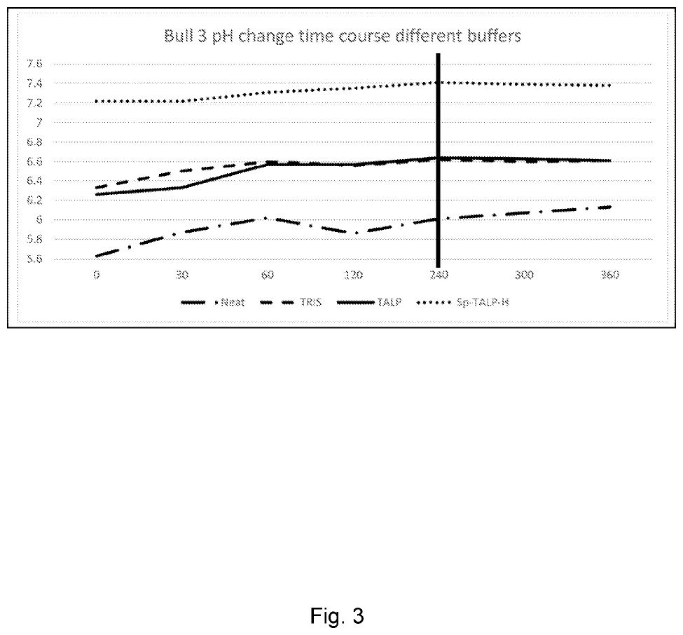

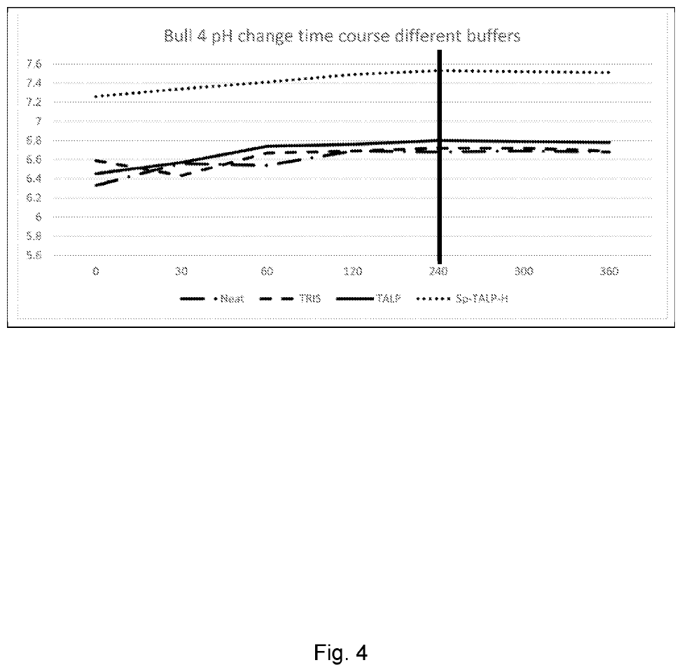

As one example, and as shown for an initial time frame in FIGS. 1-4, it may be noted that Sperm that was diluted in TRIS WS300 or left to sit at room temperature can maintain a pH range of 6.5 to 6.8 for a long period of time (˜24 hrs). Different capacitation media can have different properties and one can likely have more benefit than the other for any specific application. The goal of this experiment was to determine an initial cutoff time period and most effective pH change to alter pH and likely cleave all sialic acid groups off the Y sperm while leaving a number of X sperm available to process. This test assessed a number of variables in this experiment, namely pH over time in various buffers in neat undiluted and diluted ejaculates. An object was to quantify a change of pH in an ejaculate within a given buffer over time. From this it was shown that one step of some embodiments can be to induce capacitation, such as with heparin as in some capacitation buffers, and this step can be used to increase the pH of the overall ejaculate faster than buffers without capacitation elements.

The test protocol involved, any part of which could be used in embodiments of the present invention:
1) One ejaculate from 4 different bulls.
2) Each ejaculate was divided into four groups:
   a) Neat
   b) TRIS 300WS
   c) Clear TALP
   d) Sp-TALP-H (with heparin 10 ug/ml)
3) Because there was a little over 1 ml per ejaculate, 300 ul of ejaculate was pipetted into each of the four groups. The neat ejaculate was not diluted, whereas the other groups had 700 ul of buffer added to each tube.
4) Sampling times—allow at least 2 hrs to pass after ejaculation before processing
   a) Time 0
   b) 30 minutes
   c) 1 hour
   d) 2 hours
   e) 3 hours
   f) 4 hours
   g) 5 hours
   h) 6 hours
5) Take a pH measurement every time period indicated in the tables below. Further, the baseline pH of each buffer is shown in Table 1.

TABLE 1
pH measurements of buffers

| Buffer | Initial pH |
|---|---|
| TRIS 300 WS | 6.92 |
| Clear TALP | 7.01 |
| Sp-TALP-H | 7.44 |

Tables 2 through 5 below show the pH values in each buffer for each bull at a given time period and these are shown in FIGS. 1-4.

TABLE 2
Bull 1 - collected at 8AM sampling occurred at 11:38AM

| | | TRIS WS | Clear | |
|---|---|---|---|---|
| BULL 1 | Neat | 300 | TALP | Sp-TALP-H |
| Time 0 | 6.28 | 6.41 | 6.42 | 7.03 |

| | | | | | |
|---|---|---|---|---|---|
| 30 | minutes | 6.24 | 6.39 | 6.22 | 7.01 |
| 1 | hour | 6.26 | 6.35 | 6.31 | 7.24 |
| 2 | hours | 6.26 | 6.38 | 6.39 | 7.22 |
| 3 | hours | No Data | No Data | No Data | No Data |
| 4 | hours | 6.20 | 6.57 | 6.67 | 7.36 |
| 5 | hours | 6.23 | 6.60 | 6.66 | 7.36 |
| 6 | hours | 6.23 | 6.59 | 6.67 | 7.33 |

TABLE 3
Bull 2 - collected at 8AM sampling occurred at 11:41AM

| | | TRIS WS | Clear | |
|---|---|---|---|---|
| BULL 2 | Neat | 300 | TALP | Sp-TALP-H |
| Time 0 | 5.84 | 5.99 | 6.10 | 7.09 |

| | | | | | |
|---|---|---|---|---|---|
| 30 | minutes | 5.62 | 5.96 | 6.07 | 6.86 |
| 1 | hour | 5.72 | 6.51 | 6.44 | 7.18 |
| 2 | hours | 5.70 | 6.48 | 6.31 | 7.18 |
| 3 | hours | No Data | No Data | No Data | No Data |
| 4 | hours | 5.99 | 6.54 | 6.51 | 7.24 |
| 5 | hours | 5.84 | 6.56 | 6.51 | 7.20 |
| 6 | hours | 6.03 | 6.53 | 6.53 | 7.23 |

TABLE 4
Bull 3 - collected at 8AM sampling occurred at 11:47AM

| | | TRIS WS | Clear | |
|---|---|---|---|---|
| BULL 3 | Neat | 300 | TALP | Sp-TALP-H |
| Time 0 | 5.63 | 6.33 | 6.26 | 7.22 |

| | | | | | |
|---|---|---|---|---|---|
| 30 | minutes | 5.87 | 6.50 | 6.33 | 7.22 |
| 1 | hour | 6.02 | 6.60 | 6.57 | 7.31 |
| 2 | hours | 5.86 | 6.56 | 6.57 | 7.35 |
| 3 | hours | No Data | No Data | No Data | No Data |
| 4 | hours | 6.01 | 6.62 | 6.64 | 7.41 |
| 5 | hours | 6.07 | 6.60 | 6.63 | 7.39 |
| 6 | hours | 6.13 | 6.61 | 6.61 | 7.38 |

TABLE 5
Bull 4 - collected at 8AM sampling occurred at 11:52AM

| | | TRIS WS | Clear | |
|---|---|---|---|---|
| BULL 4 | Neat | 300 | TALP | Sp-TALP-H |
| Time 0 | 6.33 | 6.59 | 6.45 | 7.26 |

| | | | | | |
|---|---|---|---|---|---|
| 30 | minutes | 6.56 | 6.43 | 6.57 | 7.34 |
| 1 | hour | 6.54 | 6.67 | 6.74 | 7.41 |
| 2 | hours | 6.69 | 6.69 | 6.76 | 7.49 |
| 3 | hours | No Data | No Data | No Data | No Data |
| 4 | hours | 6.68 | 6.72 | 6.80 | 7.53 |
| 5 | hours | 6.69 | 6.72 | 6.79 | 7.52 |
| 6 | hours | 6.68 | 6.69 | 6.78 | 7.51 |

From this data, it can be seen that the sperm TALP buffer with added heparin is one way to increase the pH of the ejaculate by more than a desired amount, perhaps such as 1 pH unit in each ejaculate (Bull 1=1.05, Bull 2=1.4, Bull 3=1.38, Bull 4=1.18). This was reached in all ejaculates by the four-hour incubation mark. Naturally other processes and other samples can have varying times and, again, this is shown as one possible embodiment with enough information to permit those skilled in the art to readily assess how to best achieve the desired effects for their specific application within the teaching of the present invention. Noteworthy for this example, it can be seen that after an initial increase in pH, there was no significant increase in pH in any of the ejaculates after the initial time period as shown in FIGS. 1 through 4. For these initial examples, it was determined that one possible cutoff time sufficient to increase the baseline pH by a desired amount such as 0.36 units pH was between three and four hours in the Sp-TALP-H buffer. From this, it can be understood that buffers with ingredients such as heparin can induce a pH change and presumably capacitation change which can lead to a zeta charge alteration. From this initial test, it appears that the bulls had the most significant increase in pH using the Sp-TALP-H buffer among these four. The concentration of heparin in the Sp-TALP-H buffer was 10 ug/ml. This heparin concentration is also evaluated below for optimal capacitation disparity between X and Y as well as retaining viability of the X population. The increase of at least 0.36 pH units is potentially necessary for the sloughing of sialic acid groups for capacitating sperm. As noted above, this was reached in all ejaculates by the four-hour incubation mark with no increase in pH in any of the ejaculates after that time period. All of the sampling was performed at room temperature.

As mentioned, subtilities such as stress or other things can trigger the desired differential effect. Since when the needed level of the differential effect occurs can be less than immediate for some embodiments, some way of determining or measuring when is the right time to use the cells can be helpful to enable use of the invention. As mentioned, it may be desirable for the process to be timed or otherwise measured to determine when the desired differential effect exists. Not only can the level or rate of transition to the level vary based on the prior conditions of the cells or such, it can be varied based on the planned uses (immediate insemination, overnight storage, freezing, etc.) or next processing events or the like, and it can even be varied based on requirements for viability or cell function. Here, embodiments of the invention can involve determining a usable level of cell differential effect, a differential effect usable level indicator (16), a maximum differential effect difference level indicator, or a differential effect pH indicator, and affirmatively effecting that level of cell differential effect for the collection of cells, or for the sperm cell applications. Such can include determining a usable level of sperm cell sex chromosome related differential effect, and affirmatively effecting that level of sperm cell sex chromosome related differential effect for the collection of sperm cells. Determinations can be made based on a predetermined amount of time after/since a known activity (thawing, change of buffers, etc.), or they can be made based upon direct measurements or some combination of these, of course. However accomplished, embodiments can include the step of determining something, perhaps such as a maximum difference level or maximum distinction between the two types of cells, perhaps the maximum difference level of sperm cell sex chromosome related differential effect, or determining a usable pH indicated level of cell or sperm cell sex chromosome related differential effect. As explained in detail above, an increase in pH above some determined amount can be potentially necessary to enable implementation of some embodiments of the invention. Variation in pH increase determinations can include: determining a pH increase for the environment of said collection of sperm cells of determining a pH increase for the environment of said collection of sperm cells of about 0.33 pH, determining a pH increase for the environment of said collection of sperm cells of about 0.36 pH, and determining a pH increase for the environment of said collection of sperm cells of about 0.39 pH. Variation in timed differential effect determinations can include generally timing a cellular process differential transition effect for the collection of sperm cells or options including but not limited to terminating the cellular process differential transition effect immediately, terminating the cellular process differential transition effect at about 90 minutes, terminating the cellular process differential transition effect at about 120 minutes, terminating the cellular process differential transition effect at about 150 minutes, terminating the cellular process differential transition effect at up to about 150 minutes, utilizing frozen-thawed sperm cells after having been thawed for about 4 hours, utilizing frozen-thawed sperm cells after having been thawed for about 6 hours, utilizing frozen-thawed sperm cells after having been thawed for about 8 hours, utilizing frozen-thawed sperm cells after having been thawed for about 12 hours, and utilizing frozen-thawed sperm cells after having been thawed for overnight.

Returning to the example, the TRIS and clear TALP buffers maintained a pH of around 6.8 in all of the ejaculates throughout the duration of the study and therefore advantages of these for specific processes would need to be weighed against the potential that such may cause slower capacitation and thus having impacts on the separation of X from Y in a timely manner. These buffers might also be a good buffer to transfer sperm into once the capacitation reaction is completed perhaps if such were desired to be quenched as may often be desired. Quenching the induced sperm cell sex chromosome related differential effect or quenching the transition can be useful to achieving a desired cell viability. This can be achieved by a differential effect pause element (11) or pausing the effect at a desired phase such as by the step of pausing an intrinsically actionable sperm cell sex chromosome related differential effect. For example, it is possible that when the collection of sperm cells initially exists in seminal plasma, it may be desired to remove the seminal plasma from the collection of sperm cells so as to have a seminal plasma-less collection to allow their transition to occur more appropriately or perhaps even relatively immediately. In this embodiment, the step of pausing the transition can even be accomplished by the act of resubjecting the collection of sperm cells to a seminal plasma mixture (to make a seminal plasma included collection) or to BSA (bovine serum albumin) for a BSA included collection or some other appropriate substance. The aspect of pausing the differential effect can be momentary or can be complete as when pausing occurs as a stop to any further progress of the effect or transition.

All these can achieve the step of quenching capacitation of the collection of sperm cells or the element of providing a quencher (17) and embodiments can achieve subjecting the collection of sperm cells to a mixture containing substances selected from a group consisting of: seminal plasma, BSA, or the like.

In some embodiments of the present invention, sperm might be capacitated in a titrated manner perhaps to exploit differences in amino acids that may be cleaved during the capacitation process. This might reveal differences in net zeta potential charge between X bearing sperm and Y bearing sperm as well. For example, sperm could be pre-incubated such as with Sp-TALP or the like for perhaps 6 hours and 24 hours. Maturation in media such as for 24 hours, washing perhaps multiple times such as in Talp-Hepes, and in IVF-Talp and then transferal into 500 ul of IVF medium supplemented with 20 ug/ml of heparin could also be used with consideration of the specific applications as appropriate. Sperm could also be pre-incubated such as in Sp-TALP (perhaps with no heparin) such as at 39 C with 5% CO₂ for 0, 6, and 24 hours. At some point, perhaps such as the fifteen-hour mark or the like, sperm could be centrifuged and media replaced. Some period of incubation in heparin, such as a four-hour incubation with 10 ug/ml of heparin, or perhaps a four-hour incubation in heparin stock preparation of 170 units/mg dissolved in saline, with 10 ug/ml stock heparin, could be used to cause capacitation of at least bovine sperm. In addition, it is possible that shorter period of incubation could be used, for example prepurified X and Y semen were incubated in Sp-TALP-H with a heparin concentration of 10 ug/ml for three-hours at room temperature and this process showed that 50.82% of Y sperm underwent capacitation and/or death in this capacitation buffer, while only 16.88% of X sperm went through capacitation and/or death. Interestingly, the pH in both of these samples was at 7.5, while the starting pH of both samples was 6.8. Thus, as a person of ordinary skill would well understand and be able to determine for specific applications how to best apply the invention to achieve inducement of capacitation in varied manners.

As described in more detail below, ultimately, in embodiments, processes can include mixing and even incubating the collection of sperm cells with associatable particles. Particles mixed with cells can then be used as part of the separation process or modality. Here, embodiments can involve combining cells with associatable particles, and establishing a fluid combination of the associatable particles and the cells. For sperm cell applications, there can be sperm cell associatable particles with the collection of sperm cells to establish a fluid combination of sperm cell associatable particles and sperm cells. Once this combination is established, system can achieve associating a desired portion of the collection of cells or sperm cells with at least some of said associatable particles in the fluid combination. Then the step of separating at least some of the desired types of cells, perhaps the X-bearing sperm cells and Y-bearing sperm cells, can be achieved through action of the associatable particles in the fluid combination.

Having generally described the aspect of inducing capacitation to cause the desired effect, more details are presented in the following examples. Again, it should be understood that, although examples involve initial steps and even sperm cells as the initial cell item, the steps and this selection of cell or application is not intended to limit the scope of the present invention as indeed other type of cells may indeed prove valuable in applications of the general teaching of the present invention.

### Example 2: Measurement of pH of Pre-Purified X and Y Semen and Conventional Semen without Seminal Plasma in Buffers Over Time

This second example is disclosed to compare the pH change in X bearing and Y bearing sperm over time. The same buffers used in the first example were used (TRIS, Clear TALP, and the Sp-TALP-H). It shows the surprising and unanticipated differential that the two types of sperm achieve and lays a foundation for the invention whereby a difference that can be exploited and acted upon to achieve a sex related bulk separation can be created in some embodiments. As this example shows, Sp-TALP-H increased the percent capacitated Y sperm more than that of the X sperm with a heparin addition of 10 ug/ml. Naturally, other ranges of heparin concentration to induce capacitation as well as longer incubation periods to induce capacitation can be developed for each application. One goal of this experiment was to determine the cutoff time period and most effective heparin concentration to cleave all sialic acid groups off the Y sperm.

In order to determine the pH rate or amount of change in X chromosome bearing sperm versus Y chromosome bearing sperm, pre-purified X and Y semen were incubated in the aforementioned buffers over time and compared to conventional semen pH rate change. These samples were frozen thawed pre-purified X and Y sperm as was separately obtained. Additionally, capacitation rates were measured for each population with the pH increase to see how they differed from one another. Two million cells for each aliquot from each group were washed with the buffer in which they were to be incubated. Each sperm pellet was resuspended in 4 ml of the designated buffer. For each population the pH and capacitation rate or amount was measured at time 0, 30 minutes, 1 hour, 2 hours and 3 hours after incubation with the designated buffer as shown in tables 6 through 8. An object of this example is to quantify the change of pH in a given buffer from pre-purified sperm measured in different buffers over time, and also to quantify the capacitated sperm by flow cytometry correlating to the pH change and time period.

This example shows that inducing capacitation with heparin as in some capacitation buffers can increase the pH of this overall ejaculate faster than others. It also shows that this process can lead to a larger population of dead cells than seen in typical processing buffers. It supports the possibility that Y sperm may contain less sialic acid groups; therefore, the heparin inducing capacitation buffer may cause the Y sperm to capacitate more rapidly than X sperm.

This test protocol involved, any part of which could be used in embodiments of the present invention:
1) Three groups from each semen type:
   a) X (each tube contains 2 mls for a total of 4×10⁶ cells)
      i. TRIS WS 300 (2×10⁶ sperm/ml)
      ii. Clear staining TALP (2×10⁶ sperm/ml)
      iii. SP-TALP-H (2×10⁶ sperm/ml)
   b) Y (each tube contains 2 mls for a total of 4×10⁶ cells)
      i. TRIS WS 300 (2×10⁶ sperm/ml)
      ii. Clear staining TALP (2×10⁶ sperm/ml)
      iii. SP-TALP-H (2×10⁶ sperm/ml)
   c) Mixed (20×10⁶ cells divided into three groups)
      i. TRIS WS 300 (6.7×10⁶ sperm/ml)
      ii. Clear staining TALP (6.7×10⁶ sperm/ml)
      iii. SP-TALP-H (6.7×10⁶ sperm/ml)
2) Each sperm pellet was resuspended in 4 ml of the buffer they were indicated.
3) Sampling times—measure pH and fluorescence with flow cytometry
   a) Time 0
   b) 30 minutes
   c) 1 hour
   d) 2 hours
   e) 3 hours

Measurements of pH were made every time period indicated in the table below.

TABLE 6
X chromosome bearing semen.

| | | | % | | % | | % |
|---|---|---|---|---|---|---|---|
| | | TRIS | Capac- | Clear | Capac- | | Capac- |
| X semen | | WS 300 | itated | TALP | itated | Sp-TALP-H | itated |
| Time 0 | | 6.67 | 1.9 | 6.77 | 4.97 | 7.47 | 16.11 |
| 1 | hour | 6.73 | 4.12 | 6.81 | 3.54 | 7.54 | 16.6 |
| 2 | hours | 6.74 | 3.9 | 6.83 | 3.92 | 7.56 | 16.17 |
| 3 | hours | 6.74 | 4.09 | 6.82 | 4.33 | 7.58 | 16.88 |

TABLE 7
Y chromosome bearing semen.

| | | | % | | % | | % |
|---|---|---|---|---|---|---|---|
| | | TRIS | Capac- | Clear | Capac- | | Capac- |
| Y semen | | WS 300 | itated | TALP | itated | Sp-TALP-H | itated |
| Time 0 | | 6.69 | 54.35 | 6.73 | 15.81 | 7.51 | 46.11 |
| 1 | hour | 6.72 | 47.03 | 6.78 | 19.32 | 7.53 | 49.27 |
| 2 | hours | 6.72 | 45.38 | 6.77 | 18.31 | 7.54 | 45.3 |
| 3 | hours | 6.75 | 47.22 | 6.76 | 18.52 | 7.48 | 50.82 |

TABLE 8
Conventional semen (mixed X and Y sperm).

| | | | % | | % | | % |
|---|---|---|---|---|---|---|---|
| Conven- | | TRIS | Capac- | Clear | Capac- | | Capac- |
| tional | | WS 300 | itated | TALP | itated | Sp-TALP-H | itated |
| Time 0 | | 6.69 | 28.83 | 6.77 | 35.1 | 7.50 | 30.79 |
| 1 | hour | 6.72 | 18.93 | 6.77 | 31.9 | 7.57 | 28.53 |
| 2 | hours | 6.71 | 18.90 | 6.83 | 31.29 | 7.59 | 27.83 |
| 3 | hours | 6.69 | 15 | 6.81 | 32.92 | 7.58 | 28.88 |

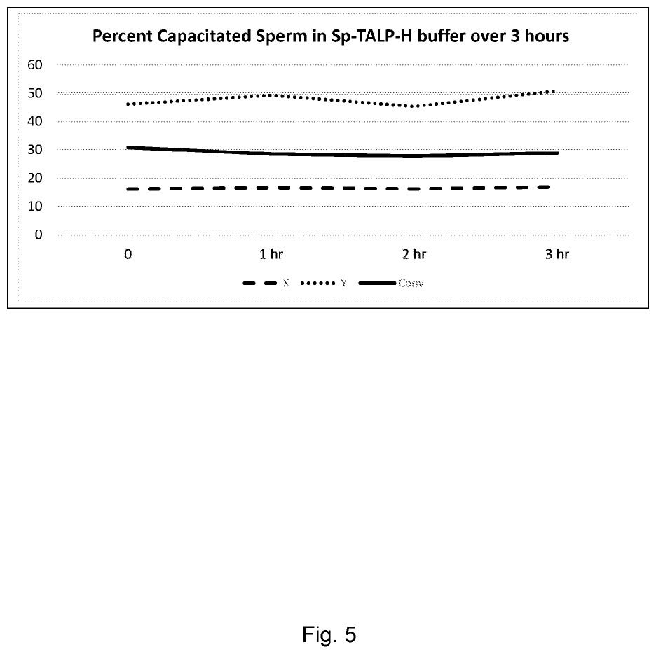

As the above data shows, surprisingly, there is a stark difference in pH and likely percent capacitation over time for X bearing sperm versus Y bearing sperm. This is shown in FIG. 5. As now disclosed, this difference in capacitation rate or amount can be used to separate the Y sperm from the X sperm. This is perhaps achieved by exploiting the overall difference in electrochemical charge on the surface of the Y sperm versus the X sperm after capacitation reaction has begun and this difference appears to be even more acute. Indeed, as shown below it may be the basis for which a total separation with complete purity can be achieved. The slower capacitation rate or total percentage of the X bearing sperm perhaps can be considered as retaining the net negative charge on the surface of the X sperm while the Y sperm lose their net negative charge perhaps by going through capacitation faster; thereby rendering the Y sperm neutral or slightly positive.

From this example, it can be seen that more Y sperm appear to be capacitated (50%) post thaw than either X sperm (17%) or conventional semen in all buffers after 3 hours. In addition, it can be seen that there is a significantly higher percent of capacitation in Y sperm than in X sperm when incubated in the Sp-TALP-H buffer. In addition, an interesting trend noticed on the dot plots generated from the flow cytometer was a change in the peaks of absolute dead sperm to more dying sperm. From this, it can be understood that the sperm might be sticking to the wall of the tube when finally dead over incubation periods. This might be utilized to refine the process for some applications. Noteworthy is that the pH may not alter that much from the buffer the frozen thawed semen in which the sperm are resuspended. This may be due to the sperm having no seminal fluid and/or buffer to alter the pH given that they are washed prior to resuspension. Further, it may be desirable to remove seminal plasma from neat ejaculates in order to control the capacitation reaction more precisely.

### Example 3: Measurement of Pre-Purified Y Semen Capacitation Via pH in Differing Concentrations of Heparin in Sp-TALP-H Buffer Over Time

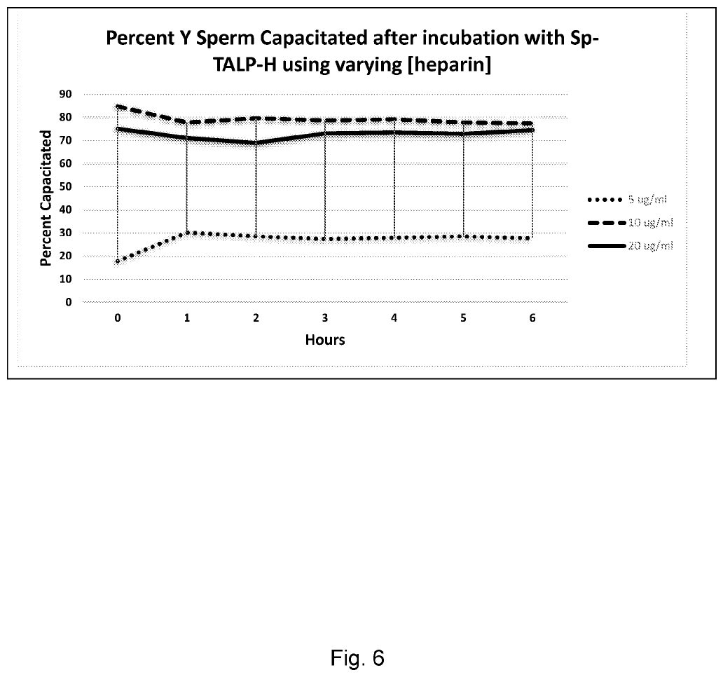

This third example is disclosed to compare the pH change in Y bearing sperm over time for differing concentrations of heparin. There are different medias that contain differing concentrations of heparin to induce capacitation. Varying concentrations of heparin were added to the Sp-TALP-H buffer and incubated with Y to determine higher or lower capacitation rates or amounts in the same amount of time. Three groups were analyzed, 4 million Y sperm each in: 1) Sp-TALP-H (5 ug/ml heparin), 2) Sp-TALP-H (10 ug/ml heparin), 3) SP-TALP-H (20 ug/ml heparin). The cells were then washed with the TRIS WS300 buffer. Each sperm pellet was resuspended in 4 ml of the buffer they were indicated. The pH and capacitation rate were measured over 6 hours as shown in Table 9 and FIG. 6.

As shown, it can be seen that by inducing capacitation such as with heparin or some otherwise appropriate capacitation buffer, the pH of the overall ejaculate can be increased faster than merely by the passage of time or the like. Such processes can also lead to a larger population of dead cells than seen in typical processing buffers. As mentioned above, it may be that Y sperm perhaps contain less sialic acid groups; therefore, the heparin inducing capacitation buffer may cause the Y sperm to have capacitated populations more rapidly than X sperm. It is also shown that the number of Y sperm capacitated may not be proportional to the concentration of heparin added to the capacitation buffer within the ranges of interest.

This test protocol involved the following processes, any part of which could be used in embodiments of the present invention:
1) Three groups:
   a) Y (each tube contains 2 mls for a total of 4×10⁶ cells)
      i. Sp-TALP-H (5 ug/ml heparin)
      ii. Sp-TALP-H (10 ug/ml heparin)
      iii. SP-TALP-H (20 ug/ml heparin)
2) Each straw was thawed and placed into a 50 ml tube. The cells were then washed with the TRIS WS300 buffer. Each sperm pellet was resuspended in 4 mls of the buffer indicated. 3) Sampling times—measure pH and measure fluorescence with flow cytometry
   a) Time 0
   b) 1 hour
   c) 2 hours
   d) 3 hours
   e) 4 hours
   f) 5 hours
   g) 6 hours

A fluorescence measurement was done by taking a 600 ul aliquot from each stock solution and adding 5 ul of propidium iodide.

TABLE 9
Capacitation rate of Y semen incubated
with varying concentrations of heparin.

| | | Sp-TALP-H | Sp-TALP-H | Sp-TALP-H |
|---|---|---|---|---|
| | | 5 ug heparin % | 10 ug heparin % | 20 ug heparin % |
| Y semen | | Capacitated | Capacitated | Capacitated |
| Time 0 | | 33.73 | 84.75 | 75.06 |
| 1 | hour | 30.12 | 77.76 | 71.06 |
| 2 | hours | 28.54 | 79.64 | 68.98 |
| 3 | hours | 27.34 | 78.70 | 73.02 |
| 4 | hours | 27.94 | 79.18 | 73.44 |
| 5 | hours | 28.50 | 77.78 | 72.80 |
| 6 | hours | 27.66 | 77.36 | 74.52 |

As can be seen, the concentration difference of heparin between 10 ug/ml and 20 ug/ml did not cause a remarkable difference in capacitation over time for these samples and these species; however, the increase of heparin from 5 ug/ml to 10 ug/ml and 20 ug/ml did make a significant difference in the percent capacitated. There is also some possibility that optimization of other buffers and the combinations of buffers may be applied in developing processes for specific applications. For example, having too large an amount of other buffer, perhaps such as TRIS buffer, remaining in the sperm might act to stabilize the pH, thus preventing or slowing capacitation. As can also be seen, in these initial examples, the amount of time does not appear to alter the percent capacitated. Considerations of a frozen thawed, washed effect might also be utilized. For example, it may be considered that the sperm may already be damaged from the sorting, freezing, thawing process and may be more susceptible to capacitation initially than Y sperm from a neat ejaculate. As those of ordinary skill in this art would well understand, this data can be used to optimize the invention for specific applications.

### Example 4: Zeta Potential Measurement of Carboxy Terminated Iron Oxide Particles in Sp-TALP-H Buffer with Either 10 ug/ml of Heparin or 20 ug/ml of Heparin

In various embodiments of the present invention, some type of separation modality can be used. These modalities can include, but are not limited to, the use of items in solution or even surfaces. An example of one type of item that can be used in solution is magnetic nanoparticles. Magnetic nanoparticles can also be associationally active with the cells to affect a separation of those cells. To the extent that the cells differentially achieve zeta potentials, magnetic nanoparticles can be used because they have a net negative zeta potential without further surface manipulation. This property can be used to bind sperm perhaps as they begin to capacitate and die. As mentioned, because sperm as they begin to capacitate have an increase in intracellular pH which appears to cause the membrane of the capacitated and/or dead sperm to lose their net negative zeta potential, the shift towards a neutral (zero) or more positive zeta potential can be used with magnetic nanoparticles. This can allow the negatively charged magnetic particles to bind specifically to those sperm that are becoming less negative and more neutral or positively charged. In general, processes can be used to titrate such differences in X bearing and Y bearing sperm.

The fourth example shows these features comparing effects for two different concentrations of heparin in the Sp-TALP buffer. It shows the zeta potential of iron nanoparticles in Sp-TALP-H in 10 ug/ml of heparin and the zeta potential of iron nanoparticles in Sp-TALP-H in 20 ug/ml of heparin. And this shows that both serve as a potential collection and/or separation mechanism. The particles demonstrate the appropriate properties desired to provide an embodiment that can be used to remove the Y population from the X bearing sperm. As mentioned, these sperm can be induced to display differential properties, potentially based on a difference in capacitation rate. This difference can be the cell's zeta charge, and in this example, the magnetic particles used to collect the capacitated sperm have an opposite zeta potential charge of one of the sperm, the collected capacitated sperm. This creates a “biological salt” to achieve separation.

As background, it may be noted that a particle with a negatively induced charge such as the carboxyl modified silane surface coating on the magnetic nanoparticles can act to bind to the membrane of the sperm through the apparent electrical charge interaction known as zeta potential. In one embodiment the fact that the material can spontaneously acquire a surface electrical charge when brought into contact with a polar medium (i.e. water) is used. Generally, an interface in deionized water is negatively charged, but there are materials that can be positively charged. An ionization of surface groups whereby a surface gains an electrical charge is observed with all metal oxide surfaces (M-OH) as well as materials that contain carboxyl and/or amino groups. This latter category includes proteins, ionic polymers, and polyelectrolytes. The ionization and/or dissociation of these groups (degree of charge development) and the net molecular charge (and thus the sign, either positive or negative) can depend strongly on the pH of the dispersion media and this can be used to advantage in embodiments of the present invention. In an embodiment of the invention, silane containing carboxyl groups may be used to perhaps result in negative zeta potential magnetic particles in capacitation buffer containing heparin perhaps such as with a pH of 7.5. These may result in the binding of capacitated and dead cells in a composition of mixed sex sample of cells. Through such processes, the Y cells may became more positively charged than the X cells in the capacitation buffer over time allowing for their removal and purification of the X sperm population.

For example, in embodiments of the present invention exploiting the zeta charge of the cells, separation can be effected with any type of magnetically identifying separating apparatus, including but not limited to devices incorporating columns, such as the Miltenyi magnetic-activated cell sorting (MACS) products, devices using simple magnetic fields applied to test tubes or containers, or other high throughput magnetic devices. As those of ordinary skill in the art would realize, any other substrate or device having a charged surface whereby the capacitated sperm may bind can also be used, including but not limited to silanes, glass surfaces, dextrans, sephacryl beads, and the like.

In considering magnetic nanoparticles for such a process, it can be understood that with the minimal difference in capacitation rate of the Y sperm in the presence of heparin at a concentration of 10 ug/ml or 20 ug/ml in Sp-TALP-H buffer, the zeta potential of iron oxide particles resuspended in either of these buffers can be used. Further, it can be understood that there is not significant zeta potential measurement difference for such particles between these two buffers. Particles that were carboxy terminated at an iron concentration such as about 20 mg/ml with a mean diameter such as about 0.67 microns can be collected magnetically. A portion, perhaps such as 2 mg can then be resuspended in 1 ml of an appropriate buffer. In this example, two such buffers were used, Sp-TALP-H, heparin 10 ug/ml and Sp-TALP-H, heparin 20 ug/ml. These were measured on a zeta sizer.

The zeta potential of these carboxyl functionalized iron oxide nanoparticles resuspended in the Sp-TALP-H buffer at either concentration of heparin showed no remarkable difference. They both have strong zeta signals which means they will not aggregate in solution but will flocculate when added to what is likely capacitating sperm to create a biological salt. Noteworthy is that the particles resuspended in Sp-TALP-H with 10 ug/ml of heparin had a mV reading of −21.1 and particles resuspended in Sp-TALP-H with 20 ug/ml of heparin had a mV reading of −23.1 so both can likely be used appropriately for these purposes.

As those of ordinary skill in the art would understand, zeta potential is described as including colloidal particles dispersed in a solution which are electrically charged due to their ionic characteristics and dipolar attributes. Each particle that is dispersed in solution can be considered as surrounded by oppositely charged ions called the fixed layer. Outside the fixed layer, there is likely varying compositions of ions of opposite polarities—forming a cloud like area. This area is called the diffuse double layer, and the whole area is often considered electrically neutral. When a voltage is applied to the dispersed particle solution, the particles are attracted to the electrode of the opposite polarity, accompanied by the fixed layer and importantly only a part of the diffuse double layer, or internal side of the “sliding surface”. This aspect suggests that some incubation to achieve the desired charge may be appropriate in particular applications. The zeta potential can therefore be considered to be the electric potential of this inner area including the conceptual “sliding surface”. As this electric potential approaches zero, particles may aggregate over a varying amount of time.

While the above particular application is explained in the context of sperm cells and in the context of zeta charge differentials, cleaving, or other surface changes, it should be understood that the invention is founded on principles that can be altered and designed for various cells, combinations with various substances, and including various separation modalities. Adaptation to particular cells, systems, or needs are within the intended scope of the invention as all should be alterations that a person of ordinary skill should be able to achieve without undue experimentation. For example, the aspect of associating a desired portion of a collection of cells with a substance is can encompass a variety of aspects within its requirement of associating. In can, of course encompass associating a desired portion of a collection of cells with a substance, associating a desired portion of cells with cell associatable particles, associating a desired portion of a collection of sperm cells with at least some sperm cell associatable particles, differentially associating a specific chromosome bearing type of sperm cell with said sperm cell associatable particles, as well as associating a desired portion of a collection of sperm cells with sperm cell associatable particles in a fluid combination, to name just a few options. The association mechanisms can vary as well to suit available substances, desired substances, varying differential effects, or the like. The association can occur due to surface effect, charge effects, chemistry, organic chemistry, or other means. It can involve electrostatically associating a desired portion of a collection of cells or sperm cells with associatable substances, particles, or surfaces, as well as chemically associating a desired portion of a collection of cells or sperm cells with substances, particles, or surfaces. Naturally the associations themselves can eb differential in that they exist for one type of cell and not another and in this manner, embodiments can involve differentially associating with the cells in the collection of cells. Charges giving rise to the association can be based upon sialic acid group content and the step of separating, such as for at least some of the X-bearing sperm cells and Y-bearing sperm cells, can be based on a sialic acid group content of the cells or sperm cells.

Associations involving combinations with antibodies (perhaps bound to particles or such) can be designed into systems as well. Importantly, while for one embodiment it is explained that likely X-bearing sperm have a larger surface area than Y-bearing sperm and therefore X-bearing sperm may have more sialic acid groups which contribute to their net negative charge prior to capacitation and death, this is not to be a limitation as to how the invention can be applied. Similarly, the explanation that as the sperm capacitate and lose their net negative charge they become positively charged in certain buffers and situations, and the Y-bearing sperm become positively charged faster than the X-bearing sperm and therefore can be collected at a time interval before X-bearing sperm become or mostly become positively charged as well are not limiting. Even embodiments focusing on steps such as having a pH change (for sperm likely at least 0.36 pH units from the initial starting pH in neat semen), there being sialic acid cleavage and/or binding of groups (other carbohydrates) that contribute net negative charge on non-capacitated living sperm, and even there being particles with an opposite charge (perhaps negative) that have an ability to bind such as to positively charged sperm as they capacitate are to be understood as only a type of embodiment that can be accomplished. Factors or variations that can be implemented in a practical application of the invention can include: a removal of sialic acid groups (as in capacitation), removal that can be accomplished with or without seminal plasma in solution, a removal that can occur naturally with time (for sperm perhaps up to 24 hours just sitting in buffer or seminal plasma), a activities that can occur at room temperature or perhaps somewhat lower, activities that can occur in a water bath up to 39 C or perhaps higher, activities that can occur just from thawing frozen cells (for example for sperm freezing does have an effect on the sperm membrane and Y-bearing sperm appear more susceptible to a faster capacitation rate post thaw potentially due to that damage), activities that can involve first freezing and then thawing to separate X-bearing and Y-bearing sperm based on capacitation rate and loss of the negatively bearing sialic acid or carbohydrate groups or such, activities where the changes such as capacitation can be induced or perhaps just amplified by certain buffers including but not limited to buffer with additives such as salts, more basic buffers (increasing the pH), Heparin additives, caffeine additives, including particles that can bind to sialic acid groups or other negatively bearing groups on the sperm surface, particles that include coating such as SNA ligands, antibodies, particles that have the opposite electrical charge (negative zeta) of capacitated sperm (positive charge).

### Example 5: Removal of Capacitated and Semen with Iron Oxide Particles in Escalating Concentrations in Sp-TALP-H Buffer with 20 ug/ml of Heparin

Of course, the ultimate goal of embodiments of the present invention is to effectively separate X and Y bearing sperm. The fifth example shows processes to achieve such a separation. In this example, Y semen was incubated with Sp-TALP-H buffer with 20 ug/ml heparin for six hours at room temperature to capacitate the sperm. An increasing amount of particles was added to the aliquots to show an increase in particles at a level that removed all capacitated and dead sperm. As background and as mentioned above, healthy viable sperm can have a net negative zeta potential. As they begin to capacitate, they can lose this negative charge or can be considered as more prone to becoming zeta resultant slightly positive and can become neutral and/or slightly positive. With negatively charged particles in a buffer which may induce capacitation of Y sperm more rapidly or to a greater degree than X sperm, the potentially more capacitated Y sperm may be attracted to the negatively charged particles or surface with a greater percentage and allow a purified X population to remain in the supernatant (nonmagnetic fraction).

This test protocol involved the following processes, any part of which could be used in embodiments of the present invention:
1) One straw of Y semen was thawed and washed to address extender present by adding 4 ml of Sp-TALP-H buffer and centrifuging.
2) The semen was resuspended in 6 ml of Sp-TALP-H buffer with 20 ug/ml heparin and BSA.
3) One ml of each resuspended cell pellet was then pipetted into six different 50 ml conical tubes.
   a) Unlabeled—control with PI
   b) 0.3 mg particles
   c) 0.6 mg particles
   d) 1.2 mg of particles
   e) 2 mg of particles
   f) 4 mg of particles
4) The appropriate amount of particles to each aliquot so that the total of the particles added equals 100 ul of the total volume.
5) Aliquots were incubated for 20 minutes.
6) After the 20-minute incubation period perhaps at room temperature (this might be done at an elevated temperature, or at some other appropriate temperature) expired for those samples containing particles, they were placed in front of a magnet for three minutes. The nonmagnetic fraction was then aspirated out of the tube and placed into a 12×75 mm FACS tube.
7) From each sample after each magnetic separation was complete, aliquots were analyzed by flow cytometry for percent capacitated prior to particle treatment as well as the percent capacitated after particle treatment by adding 10 ul of propidium iodide into the sample and looking for uptake of fluorescence.

In the above, the pre-incubation of Y sperm and X sperm was performed in the Sp-TALP-H buffer with 20 ug/ml for 3 hours. Semen was resuspended in 4 ml of Sp-TALP-H buffer with 20 ug/ml heparin and BSA, so that the final cell concentration was 160 million cells/ml. Eight milligrams of magnetic particles were removed from dH₂O, magnetically collected, and resuspended in 1 ml of Sp-TALP-H buffer with 20 ug/ml of heparin. The semen and particles were incubated for 20 minutes at room temperature. After the 20-minute incubation period had expired, for those samples containing particles—they were placed in front of a magnet to potentially induce a desired net zeta charge and also to allow migration for three minutes. The results of the aliquots analyzed by flow cytometry for the percent capacitated cell removal after particle treatment are presented in Table 10.

TABLE 10
Percent of cells removed after capacitation
with magnetic nanoparticles.

| Sample ID - | Percent Cells in | Sample ID - | Percent Cells in |
|---|---|---|---|
| Y sperm | Supernatant | X sperm | Supernatant |
| Control | 100 | Control | 100 |
| 1.2 mg particles | 0 | 1.2 mg particles | 83 |
| 2 mg particles | 0 | 2 mg particles | 80 |
| 4 mg particles | 0 | 4 mg particles | 72 |

As can be seen from Table 10, there is a noticeable and remarkable difference in the numbers of cells in the supernatant. In each of these tests, the supernatants contained diametrically opposite results for the Y versus the X sperm. No Y sperm cells remained in the supernatant, whereas virtually all of the X sperm cells (likely less either an insignificant portion or perhaps only the dead or dying cells) remained in the supernatant Likely this is due to the differing capacitation rates or total percentage of capacitated cells for Y sperm versus X sperm in the capacitation buffer with 20 ug/ml of heparin. This result was not anticipated, nor an obvious outcome. Further, when these samples are mixed, it is expected that the identical results will combine, namely, that the result will be the sum or 83/200, 80/200, 72/200 each presented as pure X sperm results with likely only some significant portion of the X sperm remaining unassociated with the magnetic nanoparticles and therefore remaining in the post magnetic separation supernatant. This will provide a truly bulk sex related separation process that can be optimized and adapted to the various species, applications, and situations according to the teachings of the present invention.

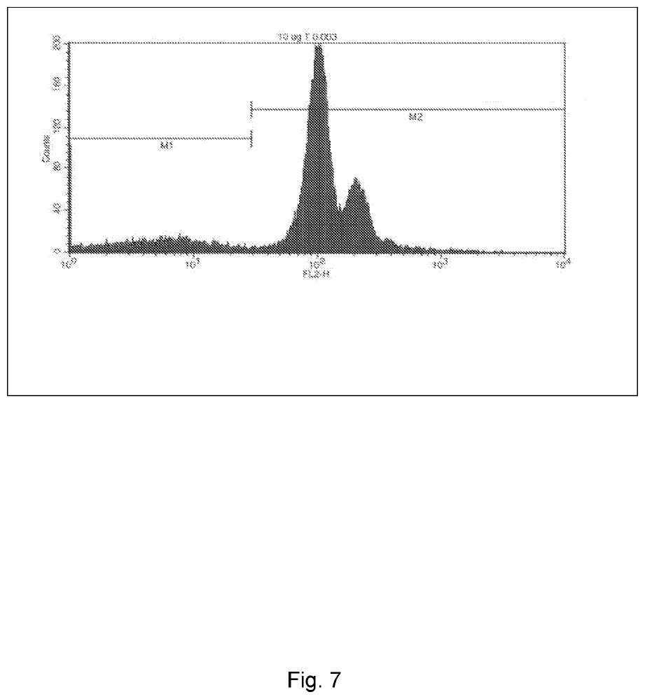

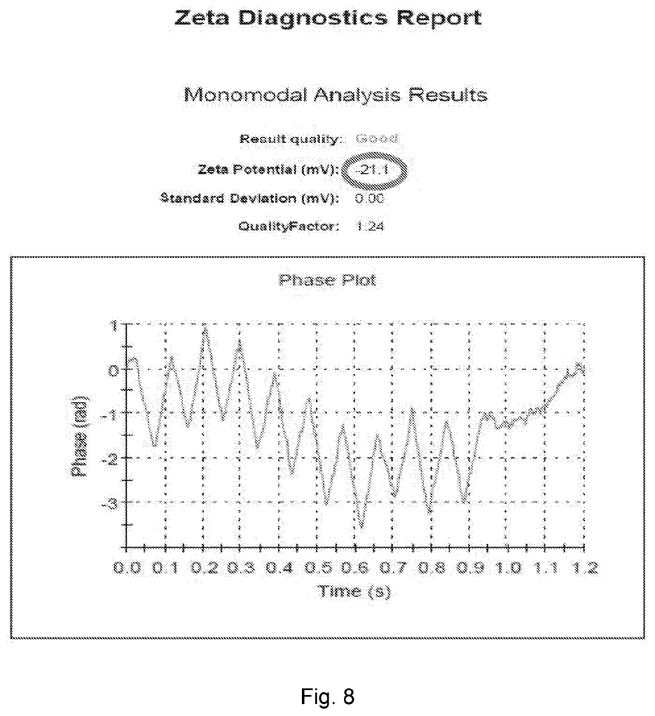

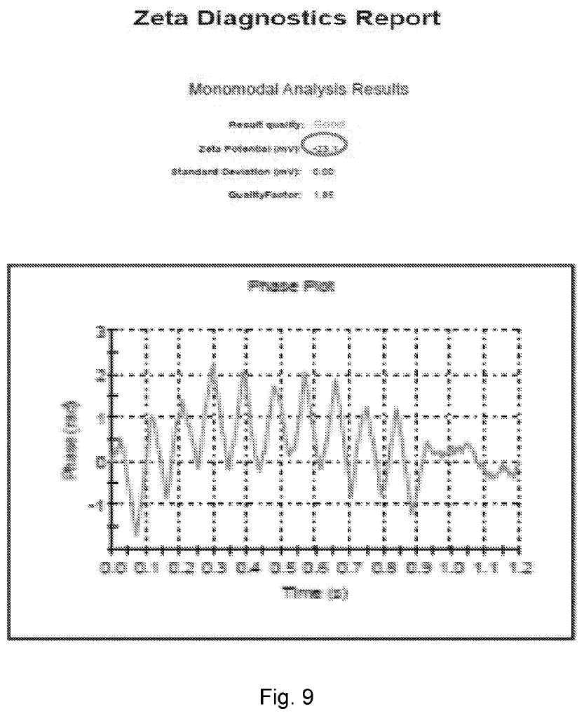

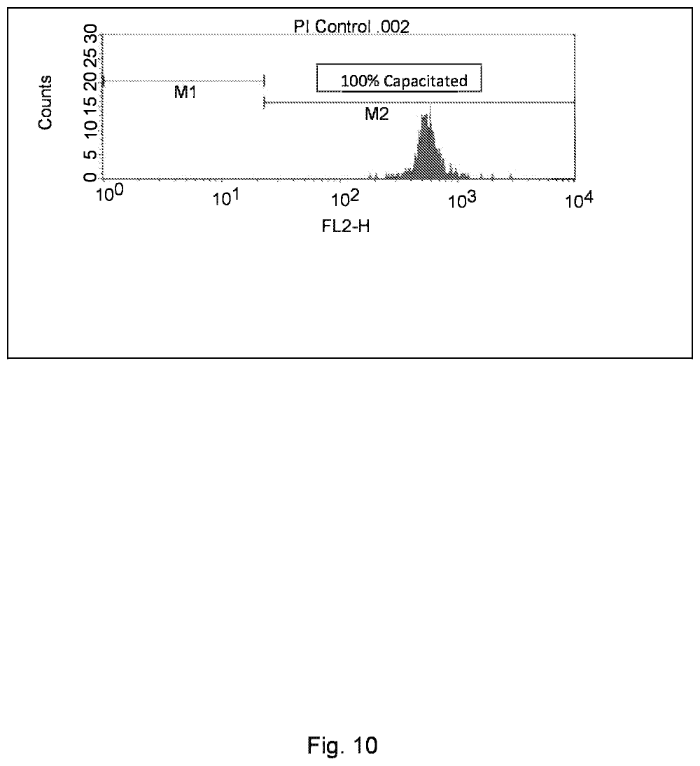

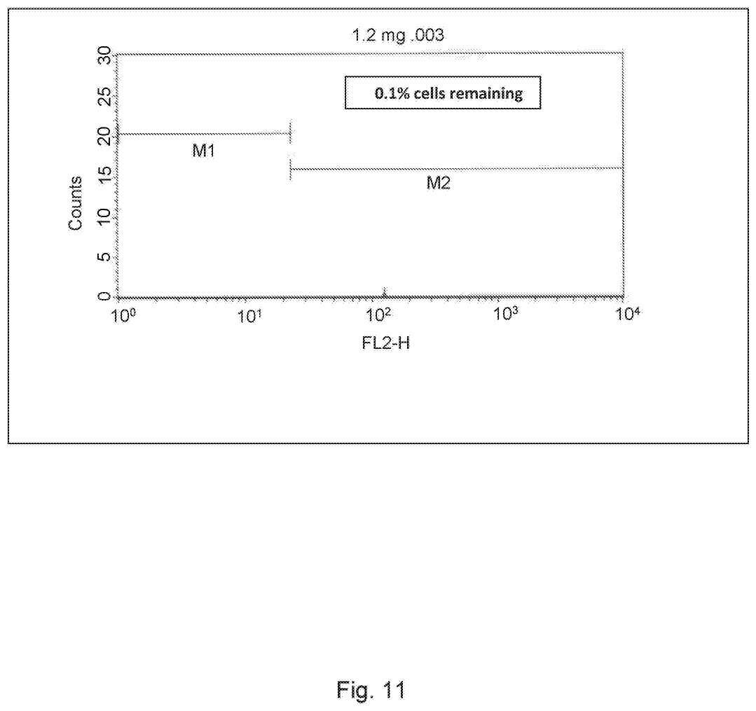

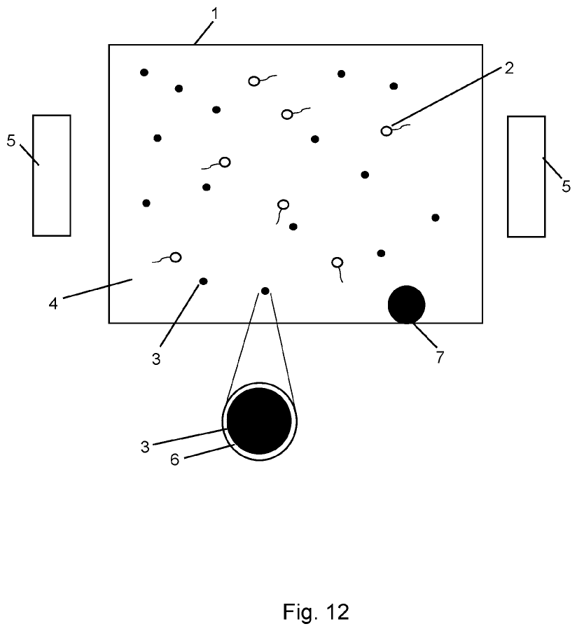

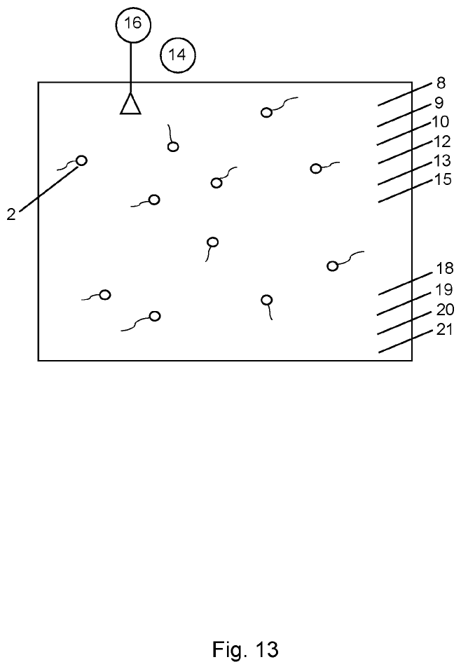

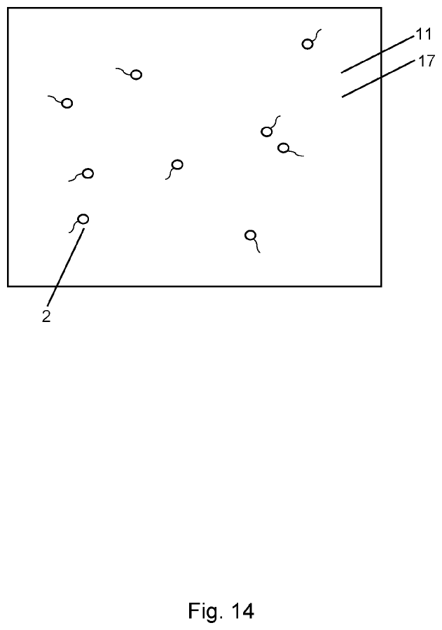

In addition, as can be seen, adding the particles at various concentrations may result in no improvement in the percent of capacitated Y sperm removed (FIGS. 7 and 8), but may result in an increase in the number of X sperm removed. A higher concentration of magnetic particles in this example may also not be necessary to remove all capacitated cells; while leaving a higher concentration of X cells available for processing and freezing for future artificial insemination use. Other substrates containing a negative zeta potential charge or surfaces containing an external electric charge potential can be used for the removal of these pretreated cells. To effect a separation of the two different types of cells, different mechanisms are possible. In contrast to the non-enabled techniques proposed whereby the different sexed sperm ought to simply swim apart or turn into some type of a 2-phase liquid, embodiments of this invention generally involve an external force or the like as the separation modality. Such can include separating such as magnetically separating at least some of one type of cells from the collection of cells, electrostatically separating at least some of one type of cells from the collection of cells, separating at least some of one type of cells from the collection of cells by electrophoresis, gravimetrically separating at least some of one type of cells from said collection of cells (perhaps with the right particle or such), and separating at least some of one type of cells from the collection of cells by centrifugation. Thus for sperm, the step of separating at least some of said X-bearing sperm and Y-bearing sperm cells can be through action of the sperm cell associatable particles in the fluid combination of sperm cell associatable particles and sperm cells where the particles (to which a desired portion of the cells are bound) are what is moved by the external force to effect the separation and thus the step of separating is through action of the cell or sperm cell associatable particles.

As mentioned above, where particles are used as a separation modality, embodiments can involve combining sperm cell associatable particles with the collection of sperm cells to establish a fluid combination (4) of sperm cell associatable particles and sperm cells. Similarly, a system can involve a plurality of sperm cell sex chromosome differentially associatable particles (3) fluidically combined with the collection of sex chromosome differential exhibiting sperm cells so as to establish a fluid combination of sperm cell sex chromosome differentially associatable particles and sex chromosome differentially exhibiting sperm cells. The particles can be nano sized such as might establish a suspension or even micro sized such as might establish a settle-able mixture or such. Here, the process can involve combining cell or sperm cell associatable nanoparticles with the collection of cells or sperm cells to establish a fluid combination of cell or sperm cell associatable nanoparticles and sperm cells which can then be separated.

The sizes can be varied and designed as desired. Using sperm cells as but one example, systems can involve sperm cell associatable nanoparticles, particles can be: sperm cell associatable particles having an mean diameter of between 10 nm and 999 nm, sperm cell associatable microparticles, sperm cell associatable particles having an mean diameter of between 100 nm and 100 um, sperm cell associatable particles having an mean diameter of not more than about 1000 nm, sperm cell associatable particles having an mean diameter of about 670 nm, sperm cell associatable particles having an mean diameter at least about 100 nm, sperm cell associatable particles having an mean diameter at least about 300 nm, sperm cell associatable particles having an mean diameter at least about 500 nm, sperm cell associatable particles having an mean diameter at least about 600 nm, of the like.

Particles can be made of a great variety of substances such as: iron oxide particles, glass particles, silica particles (sol-gel), silica with aluminum substitution particles (such as can be employed to increase the negative colloidal charge, especially when it is evaluated at pH below the neutral point perhaps because of their very small size, the surface area of colloidal silica is very high), borosilicate particles, plastic particles, PVP particles, styrofoam, polyvinlypropylene particles, polyvinylpyrrolidone particles, polystyrene particles, melamine particles (polymethylenemelamine nanospheres and microspheres are made from crosslinked melamine and can have some advantages depending on the application compared to polystyrene particles: they can have a higher density (˜1.51 g/cm³), can be very stable, can be stored indefinitely, can be resuspended in water, do not swell or shrink in most organic solvents, and can be heat resistant up to 300° C. Further, the surface of plain melamine microparticles are often terminated with methylol groups, which could be readily functionalized in a desired manner), PMMA particles, (polymethymethacrylate is a synthetic resin produced from the polymerization of methyl methacrylate, represents a transparent and rigid plastic), polylactide particles (poly or polylactic acid or polylactide is a biodegradable and bioactive thermoplastic aliphatic polyester derived from renewable resources, such as corn starch, cassava roots, chips or starch, or sugarcane that at one time had the second highest consumption volume of any bioplastic of the world), particles bound to polar molecules (so as to change the zeta potential to bind to cells that are positively charged), dextran particles, functionalized surface particles, magnetic or such particles coated with any of the above materials, or functionalized surfaces for all the above particles. These particles can be mobile in the fluid, can be as a packed column perhaps such as micro or micron sized particles used in packed columns, or as an item that can achieve settling in a solution.

Further, the invention includes the aspect of achieving artificial insemination and production of animals using the resultant products. Artificial insemination using semen with this type of preselected sex could have several potential benefits such as: higher production levels with reduced costs, improvement in animal health and welfare, reduction of environmental impact due to the elimination of the unwanted sex before they grow to adulthood, and faster genetic progress, and the like. The present invention may provide a more commercially viable technique with advantages such as: (i) capital equipment intensive free, to reduce costs and exclude the need for highly trained technicians; (ii) non-destructive, to avoid any vitality alteration of the separated sperm; (iii) bulk separations to produce more separated sperm in a shorter amount of time.

As mentioned, embodiments can involve processes such as proximally situating a substance in the vicinity of a collection of sperm cells; associating a desired portion of the collection of sperm cells with a substance; and separating types of cells, perhaps X-bearing sperm cells and Y-bearing sperm cells, through action of the substance. As mentioned, embodiments can establish a packed column, suspension, a mixture, or such. For a packed column, the column may be flooded with a buffer inducing a net negative charge on the surface of the particles. X-bearing sperm would be collected as the effluent using either gravity or pressure to force the fluid through the column. A full range of option are available for a system design including involving: suspending sperm cell associatable particles with a collection of sperm cells to create a suspension of sperm cell associatable particles in a collection of sperm cells, combining likely heavier or larger sperm cell associatable particles with a collection of sperm cells to create an unsuspended collection of sperm cell associatable particles in the collection of sperm cells, mixing sperm cell associatable particles with a collection of sperm cells to create a mixture of sperm cell associatable particles in the collection of sperm cells, moving sperm cell associatable particles through a collection of sperm cells, employing a packed column that may provide a sperm cell passageway past said sperm cell associatable particles or sperm cell associatable particles passageway past said sperm cells, and moving a collection of sperm cells through the sperm cell associatable particles. Larger particles can be employed to specifically avoid establishing a suspension and even forcing the particles to settle out once associated and here cell or perhaps sperm cell associatable particles having a mean diameter at least about 1000 nm can be used.

The particles, surfaces, or substances can be manufactured with, perhaps coated with, a desired substance so as to be coated sperm cell associatable particles. These coated or otherwise treated particles can include a great variety of substances to suit the particular application, including but not limited to: carboxyl modified silane coated sperm cell associatable particles, carbohydrate coated sperm cell associatable particles, ligand coated sperm cell associatable particles, Sambucus nigra agglutinin (SNA) coated sperm cell associatable particles, Monosaccharide coated sperm cell associatable particles, antibody coated sperm cell associatable particles, polymerase associatable particles, receptor molecule associatable particles, Cas9-type associatable particles, CRISPR-type associatable particles, DNA tag associatable particles, sperm cell differentiatable condition active sperm cell associatable particles, or the like. They can be manufactured using known techniques so the steps of combining can involve: combining particles stabilized by pH adjustment with a collection of sperm cells to establish a fluid combination of sperm cell associatable particles and sperm cells, combining a pH particle combination stabilized particle containing fluid with a collection of sperm cells to establish a fluid combination of sperm cell associatable particles and sperm cells, combining a particle concentrated particle fluid with a collection of sperm cells to establish a fluid combination of sperm cell associatable particles and sperm cells, combining a stable particle fluid with a collection of sperm cells to establish a fluid combination of sperm cell associatable particles and sperm cells, combining a stable concentration level particle fluid with a collection of sperm cells to establish a fluid combination of sperm cell associatable particles and sperm cells, combining a particle size-concentration level coordinated stable particle fluid with a collection of sperm cells to establish a fluid combination of sperm cell associatable particles and sperm cells, combining a 50 nm particle size-50% wt solids particle fluid with a collection of sperm cells to establish a fluid combination of sperm cell associatable particles and sperm cells, combining a 10 nm particle size-30% wt solids particle fluid with a collection of sperm cells to establish a fluid combination of sperm cell associatable particles and sperm cells, or the like. In manufacture, the colloidal or other suspension can be stabilized by pH adjustment and then concentrated, perhaps by evaporation. The maximum concentration obtainable can depend on the particle size. For example, 50 nm particles can be concentrated to greater than 50 wt % solids while 10 nm particles can only be concentrated to approximately 30 wt % solids before the suspension becomes too unstable.

In some embodiments of a bulk process, targeted, perhaps capacitated, cells labeled with ‘charged’ magnetic particles and subjected to magnetic cell separation in an ‘open’ (columnless) magnetic system can be removed more efficiently and in greater numbers per time unit compared to flow cytometry. Magnetic cell separation requires no internal operating pressure or if pressurized, a lower internal operating pressure and the stream of fluid containing the cells does not have to be broken into cell damaging droplets as that for flow cytometry. Further, the sheath fluid required for flow cytometry is generally a salt-based, lipo-protein deficient physiological medium. Magnetic cell separation can allow some cells, such as sperm in but one, non-limiting example, to be bathed in nutrient-rich buffers that may promote and prolong cell viability during the separation procedure. Further, for some embodiments and applications, it may also be beneficial to separate the cells in a buffer, at a temperature, or cause situations that otherwise might render them less mobile or even immobile for a time so that normal swimming does not impact a desired separation process. In other cell separation applications, embodiments of the present invention can be used to magnetically label and ultimately remove capacitated sperm and/or Y sperm through magnetic cell separation procedures. The resultant desired sub-population of harvested cells can be selected for viable X sperm as well as for specific cellular attributes.

As mentioned throughout, the present invention can be adapted in a broad number of ways. As just one type of the many examples of adaptation of these teachings, even the previously tried but unsuccessful Percoll method might be an adaptation. As should be understood, Percoll might present another type of associationally active element. It may not have worked before because Percoll with the buffer in it is a negatively charged environment. It may have removed the dead and dying sperm but not altered sex ratio unless it were applied in a manner according to the present invention whereby the sperm were appropriately or perhaps forcibly capacitated such as with a capacitation agent to increase the pH and to affirmatively establish the differential effect. With this, it is likely that one could even adapt Percoll to separate X from Y.

As mentioned above, the viability of the cells can be important—especially for sperm cells. For this aspect, embodiments of the invention can involve selectively impacting substantially only one type of cells from said collection of cells while leaving the other type of cells from said collection of cells substantially unimpacted, such as, for sperm cells, selectively impacting substantially only one type of sex chromosome bearing sperm cells from said collection of sperm cells while leaving the other type of sex chromosome bearing sperm cells from said collection of sperm cells substantially unimpacted, and acting on at least a portion of said only one type of sex chromosome bearing sperm cells that have been selectively impacted. By acting on only one type of cell (e.g., unlike the flow cytometry act of staining both types), the other, perhaps desired type of cell is nearly totally unimpacted through processes that cause no substantial effect on one type of cells from the collection of cells. Those unaffected cells can only have natural effects and by the act of subjecting that one type of cells from the collection of cells to only natural effects they exist in a better condition. This can be done by avoiding actions on or condition for such cells including but not limited to steps such as: avoiding associating any unnatural substance with one type of cells from the collection of cells perhaps, avoiding staining one type of cells from the collection of cells, avoiding subjecting one type of cells from the collection of cells to any non-naturally occurring external forces, non-cell motility separating at least some of one type of cells from the collection of cells, avoiding hyperactivity in one type of cells from the collection of cells such as can be caused by the acrosome reaction of the like, avoiding any physical activity for one type of cells from the collection of cells so they are not stressed, tired, or weakened. It can also be done by providing a no unnatural substance for one type of cells collection of cells (18), a no stain collection of cells (19), a no hyperactivity collection of cells (20), a no physical activity collection of cells (21), or the like.

In considering the example of embodiments involving avoiding subjecting one type of cells from the collection of cells to any non-naturally occurring external forces, it can be interesting to note that if designed with some of the disclosed separation modalities, the step of separating at least some of one type of cells from the collection of cells can occur while acting substantially passively with respect to another type of the cells. The external force or external force separation modality acts on the type of cells associated with particles, not the other type of cells and so when external force separating at least some of one type of cells from the collection of cells, the other type of cells is substantially undisturbed. Once acted upon to achieve separation, embodiments can capture a desired (or for that matter in the overall use scheme an undesired) type of cells in the collection of cells or can provide a desired type of cell capture element. All of these aspects can contribute to eventually providing, as an end result of separation, cells that are more viable which is certainly a desire for sperm cells to be used in fertilization processes.

This feature of embodiments of the invention can be critical to ultimate application of the invention. Using sperm cells as one example, the initial collection of cells may be established with both the X-bearing and Y-bearing sperm cells in the collection prior to separation as being functionally viable X-bearing and Y-bearing sperm cells. These cells should be functionally viable cells that are usable in practical application for fertilization processes be them AI, IVF, or otherwise. For artificial insemination purposes, both going in and coming out of the overall separation system, the desired cells should be functionally viable cells usable in practical application for artificial insemination processes. Even the removed cells should be cells, at least prior to being associated and separated, that are or were functionally viable cells usable in practical application for artificial insemination processes. Allowing capacitation to proceed too far, as one example, as when in the acrosome reaction, can destroy viability in the sense that the cells will have substantial losses, and so embodiments of the invention can require that both the X-bearing and Y-bearing sperm cells in the collection prior to separation be sperm cells in a state where they are practically usable for fertilization without substantial loss of those cells. Thus, embodiments can include using pre-acrosome reaction initiation sperm cells. Other embodiments of the invention can, of course, be applied to remove non-viable cells and so embodiments can include dying or functionally impaired cells, perhaps such as sperm cells, in novel ways. However, using viable cells in general can include having the cells, perhaps sperm cells, in a state where they are or would be viable in the senses stated above: after overnight storage, after shipping in a natural state, after freezing, shipping and thawing, after having been frozen, after having been frozen and then thawed, after at least about 8 hours for cells held in an unfrozen state in seminal plasma, after at least about 16 hours for cells held in an unfrozen state in seminal plasma, after at least about 24 hours for cells held in an unfrozen state in seminal plasma, after at least about 30 minutes after thawing for cells frozen and then thawed, after at least about 45 minutes after thawing for cells frozen and then thawed, after at least about 1 hour after thawing for cells frozen and then thawed, after at least about 2 hours after thawing for cells frozen and then thawed, cells that remain practically usable for fertilization without a loss of more than 20% of such cells, cells that remain practically usable for fertilization without a loss of more than 30% of such cells, cells that remain practically usable for fertilization without a loss of more than 40% of such cells, or the like.

As mentioned, control and repeatability of embodiments, and perhaps viability, can be accomplished by timing such as of a differential transition effect for the collection of cells and for sperm cells experiencing a capacitation effect, values can include using and acting on: sperm cells less than 90 minutes after having been subjected to a capacitation change effect, sperm cells less than 120 minutes after having been subjected to a capacitation change effect, sperm cells less than 150 minutes after having been subjected to a capacitation change effect, sperm cells less than 180 minutes after having been subjected to a capacitation change effect, sperm cells less than 210 minutes after having been subjected to a capacitation change effect, sperm cells less than 90 minutes after having been subjected to a heparin activation, sperm cells less than 120 minutes after having been subjected to a heparin activation, sperm cells less than 150 minutes after having been subjected to a heparin activation, sperm cells less than 180 minutes after having been subjected to a heparin activation, sperm cells less than 210 minutes after having been subjected to a heparin activation, sperm cells in a state of from 180-240 minutes after having been subjected to a heparin, activation, or the like.

Further, to aid in assuring the cells are as viable as possible, embodiments can involve maintaining the cells, perhaps sperm cells, in a nurturing environment throughout all or mostly all of the steps of the separation system. In this way, systems can provide one or more nurturing environments (8) for said cells that exist in all aspects of the system. This can involve avoiding subjecting the cells to buffers, conditions (e.g., for sperm cells even isolating those cells from the collection such as in flow cytometry), or other influences that are less than optimal, not generally or universally accepted as safe or non-detrimental, or perhaps just undesired by users. In this regard it may be noted that for sperm cells, using heparin—at least at the initial stages of capacitation such as with pre-acrosome reaction initiation sperm cells—is generally accepted as non-detrimental as merely causing the natural process of capacitation at controlled times external to a natural fertilization process. Nurturing environments can involve including a protein source mixed with the sperm cells throughout all or most of the steps or one or more protein sources (9) for cells that exist in all aspects of the system.

From the above disclosure, it should be understood that the present invention encompasses fundamental processes as well as specific implementations and adaptations that may be developed by a mere application of ordinary skill to the teachings contained herein. An overall process and all reasonable adaptations using these teachings should be understood as covered by this initial disclosure. With this understanding, one more general embodiment of the current process can involve some combination of the following steps: 1) obtaining a sample; 2) incubating the sample in an appropriate buffer to induce a desired differential effect; 3) suspending associationally active particles in a similar or perhaps identical buffer; 4) mixing an aliquot of suspended particles with suspended cells at desired ratios; 5) incubating the mixture for a desired period of time; 6) subjecting the mixture to a separation modality for a set period of time; and 7) separating, perhaps by removing, a portion of the mixture so as to yield a desired resultant selected portion of the total. In addition, a more detailed embodiment can include aspects such as: a) pre-incubation of Y sperm and X sperm in the Sp-TALP-H buffer perhaps such as with 20 ug/ml for 3 hours; b) resuspension of the cells perhaps such as in 4 ml of Sp-TALP-H buffer with 20 ug/ml heparin and BSA; c) achieving a desired cell concentration perhaps such as 160 million cells/ml; removing from dH₂O or the like, magnetically collected particles; d) resuspension of iron oxide nanoparticles perhaps such as in 1 ml of Sp-TALP-H buffer with 20 ug/ml of heparin; e) adding an appropriate amount of particles to an aliquot perhaps such as so that the total of the particles added equals 100 ul of the total volume; incubating the semen and particles perhaps such as for 20 minutes at room temperature; f) placing the mixture in front of a magnet to potentially induce a desired net zeta charge and also to allow migration perhaps such as for three minutes; g) aspirating out the nonmagnetic fraction; and h) using that fraction for desired purposes perhaps such as for artificial insemination. Of course, additional optional steps disclosed above throughout this disclosure can be used or substituted, including but not limited to using and even thawing a frozen semen amount to obtain the sample prior to achieving separation on the thawed sample, and the like.

As mentioned, it should be understood that the principles of the invention can be applied in a great variety of adaptations. Systems can be designed without undue experimentation for sperm cells, and for non-sperm cells that exhibit appropriate properties. Using just the subset of cells that are sperm cells as but one example, embodiments can be designed and adapted to involve selecting sperm cells that have never been frozen for the collection of sperm cells, selecting sperm cells that have been frozen and then thawed for the collection of sperm cells, selecting human sperm cells for the collection of sperm cells, selecting non-human sperm cells for the collection of sperm cells, selecting bovine sperm cells for the collection of sperm cells, selecting porcine sperm cells for the collection of sperm cells, separating at least some X-bearing sperm cells and Y-bearing sperm cells. Particular embodiments can involve using: sperm cells, X-bearing sperm cells and Y-bearing sperm cells, X-bearing sperm cells and Y-bearing sperm cells for artificial insemination use, dying or functionally impaired sperm cells, and even dying or functionally impaired cells in general.

One aspect where the invention provides a long desired, but never repeatable or enabled to be achieved, feature is that of providing a bulk separation process especially for sexed sperm cells. For sexed sperm cells, by providing the advantages of speed of separation and cost of separation, bulk separating the cells has the potential of literally changing the industry as compared to the only practical, repeatable, enabled sex separation process, namely, flow cytometry processing which is done one-by-one and with rather harsh treatment of the sperm cells. As a bulk separation process, embodiments of the invention can involve acting on at least some of the collection of sperm cells in a manner that allows bulk separating at least some of the X-bearing sperm cells and Y-bearing sperm cells based upon said sperm cell sex chromosome related differential effect. Further, such as for the viability concerns mentioned above, the act of bulk separating cells can be accomplished while the cells are still in the collection of cells. Further, bulk separating the cells can involve separating the cells more than one-at-a-time, simultaneously separating a significant quantity of said cells in the collection of cells, simultaneously separating the majority of the desired type of said cells in the collection of cells, simultaneously separating substantially all of the desired type of said cells in the collection of cells, simultaneously separating at least ten thousand of the cells at a time, a more than one-at-a-time separation modality, a simultaneous significant quantity cell separation modality, a simultaneous majority of the desired type of cell separation modality, a simultaneous substantially all of the desired type of cell separation modality, an at least ten thousand of said cells at a time simultaneous cell separation modality, or the like.

These bulk processes can even be accomplished to repeatably to separate at remarkable purities both for the new existence of a bulk process and even as compared to the flow cytometry-based processes. These can include purities such as: separating substantially all of a desired type of said cells in said collection of cells, separating substantially all of a desired type of said cells in said collection of cells, separating at least about 70% of a desired type of said cells in said collection of cells, separating at least about 80% of a desired type of said cells in said collection of cells, separating at least about 90% of a desired type of said cells in said collection of cells, separating at least about 95% of a desired type of said cells in said collection of cells, separating at least about 97% of a desired type of said cells in said collection of cells, separating at least about 98% of a desired type of said cells in said collection of cells, separating at least about 99% of a desired type of said cells in said collection of cells, separating so as to leave no appreciable viable cells of a desired type of said cells in said collection of cells, separating at flow cytometry achievable levels of purity, or the like.

As mentioned, gravimetric processes can be used for the step of separating the cells. This can involve combining cell associatable particles with the collection of cells to establish a fluid combination of cell associatable particles and sperm cells, associating a desired portion of the cells with the cell associatable particles, and gravimetrically separating at least some of the cells through action of the cell associatable particles. As can be appreciated, embodiments can involve gravitationally separating at least some of one type of cells from the collection of cells. It can also include enhanced force separating at least some of one type of cells from the collection of cells, perhaps such as or also, centrifugationally separating at least some of one type of cells from the collection of cells, and separating at least some of the cells by centrifugation. Such processes can involve using cell associatable microparticles, a gravimetric separation modality (7) and gravimetrically separating with a gravimetric force greater than any cell motility force, a greater than cell motility effect gravimetric cell separation modality, and even cell associatable particles having a mean diameter at least about 1000 nm so as to not be suspended but to more likely settle out or be gravimetrically forced out of the collection of cells.

While the invention has been described in connection with some preferred embodiments, it is not intended to limit the scope of the invention to the particular form set forth, but on the contrary, it is intended to cover such alternatives, modifications, and equivalents as may be included within the spirit and scope of the invention as defined by the statements of inventions. Examples of alternative claims may include:
1. A method for separation of X-bearing sperm cells and Y-bearing sperm cells comprising the steps of:
   establishing a collection of sperm cells that has both X-bearing sperm cells and Y-bearing sperm cells in said collection;
   inducing a sperm cell sex chromosome related differential effect in said collection of sperm cells;
   combining sperm cell associatable particles with said collection of sperm cells to establish a fluid combination of sperm cell associatable particles and sperm cells;
   associating a desired portion of said collection of sperm cells with at least some of said sperm cell associatable particles in said fluid combination of sperm cell associatable particles and sperm cells;
   separating at least some of said X-bearing sperm cells and Y-bearing sperm cells through action of said sperm cell associatable particles in said fluid combination of sperm cell associatable particles and sperm cells.
2. A method for separation of X-bearing sperm cells and Y-bearing sperm cells comprising the steps of:
   establishing a collection of sperm cells that has both X-bearing sperm cells and Y-bearing sperm cells in said collection;
   proximally situating a substance in the vicinity of said collection of sperm cells;
   associating a desired portion of said collection of sperm cells with said substance; and
   separating at least some of said X-bearing sperm cells and Y-bearing sperm cells through action of said substance.
3. A method for separation of X-bearing sperm cells and Y-bearing sperm cells comprising the steps of:
   establishing a collection of sperm cells that has both viable X-bearing sperm cells and viable Y-bearing sperm cells in said collection;
   affirmatively supporting an intrinsically actionable sperm cell sex chromosome related differential effect for said collection of sperm cells;
   allowing said intrinsically actionable sperm cell sex chromosome related differential effect to exist in said collection of sperm cells without compromising use of any of such sperm cells for fertilization processes;
   acting on said intrinsically actionable sperm cell sex chromosome related differential effect in said collection of sperm cells; and
   separating at least some of said X-bearing sperm cells and Y-bearing sperm cells based upon said intrinsically actionable sperm cell sex chromosome related differential effect.
4. A method for separation of X-bearing sperm cells and Y-bearing sperm cells comprising the steps of:
   establishing a collection of sperm cells that has both X-bearing sperm cells and Y-bearing sperm cells in said collection;
   differentially associating with sperm cells in said collection of sperm cells based upon sialic acid group content; and
   separating at least some of said X-bearing sperm cells and Y-bearing sperm cells based on said sialic acid group content of said sperm cells.
5. A method for separation of X-bearing sperm cells and Y-bearing sperm cells comprising the steps of:
   establishing a collection of sperm cells that has both X-bearing sperm cells and Y-bearing sperm cells in said collection;
   selectively impacting substantially only one type of sex chromosome bearing sperm cells from said collection of sperm cells while leaving the other type of sex chromosome bearing sperm cells from said collection of sperm cells substantially unimpacted;
   acting on at least a portion of said only one type of sex chromosome bearing sperm cells that have been selectively impacted; and
   separating at least some of said X-bearing sperm cells and Y-bearing sperm cells.
6. A method for bulk separation of X-bearing sperm cells and Y-bearing sperm cells comprising the steps of:
   establishing a collection of sperm cells that has both X-bearing sperm cells and Y-bearing sperm cells in said collection;
   acting on at least some of said collection of sperm cells based on a sex differential property of said sperm cells; and
   bulk separating at least some of said X-bearing sperm cells and Y-bearing sperm cells based upon said sperm cell sex chromosome related differential property.
7. A method for separation of cells comprising the steps of:
   establishing a collection of cells;
   combining cell associatable particles with said collection of cells to establish a fluid combination of cell associatable particles and cells;
   associating a desired portion of said cells with said cell associatable particles; and
   gravimetrically separating at least some of said cells through action of said cell associatable particles.
8. A method for bulk separation of cells comprising the steps of:
   establishing a collection of cells;
   inducing a differential effect for said establishing a collection of cells to establish a collection of differentially exhibiting cells;
   acting on at least some of said collection of differentially exhibiting cells based on a differential property of said cells; and
   bulk separating at least some of said cells based upon said differential property.
9. A method of separating cells as described in clauses 1, 2, 3, 4, 5, 6, or any other clause, wherein both said X-bearing and Y-bearing sperm cells in said collection prior to separation comprise functionally viable X-bearing and Y-bearing sperm cells.
10. A method of separating cells as described in clause 9, or any other clause, wherein said functionally viable X-bearing and Y-bearing sperm cells comprise functionally viable cells usable in practical application for fertilization processes.
11. A method of separating cells as described in clause 10, or any other clause, wherein said functionally viable cells usable in practical application for fertilization processes comprise functionally viable cells usable in practical application for artificial insemination processes.
12. A method of separating cells as described in clause 9, or any other clause, wherein both said X-bearing and Y-bearing sperm cells in said collection prior to separation comprise sperm cells in a state where they are practically usable for fertilization without substantial loss of such cells selected from a group consisting of:
   after overnight storage,
   after shipping in a natural state,
   after freezing, shipping and thawing,
   after having been frozen,
   after having been frozen and then thawed,
   after at least about 8 hours for cells held in an unfrozen state in seminal plasma,
   after at least about 16 hours for cells held in an unfrozen state in seminal plasma,
   after at least about 24 hours for cells held in an unfrozen state in seminal plasma,
   after at least about 30 minutes after thawing for cells frozen and then thawed,
   after at least about 45 minutes after thawing for cells frozen and then thawed,
   after at least about 1 hour after thawing for cells frozen and then thawed,
   after at least about 2 hours after thawing for cells frozen and then thawed,
   cells that remain practically usable for fertilization without a loss of more than 20% of such cells,
   cells that remain practically usable for fertilization without a loss of more than 30% of such cells,
   cells that remain practically usable for fertilization without a loss of more than 40% of such cells,
   and all permutations and combinations of each of the above.
13. A method of separating cells as described in clause 9, or any other clause, wherein said functionally viable X-bearing and Y-bearing sperm cells comprise capacitation triggered sperm cells.
14. A method of separating cells as described in clause 13, or any other clause, wherein said capacitation triggered sperm cells comprise pre-acrosome reaction initiation sperm cells.
15. A method of separating cells as described in clause 13, or any other clause, wherein said capacitation triggered sperm cells comprise sperm cells selected from a group consisting of:
   sperm cells less than 90 minutes after having been subjected to a capacitation change effect,
   sperm cells less than 120 minutes after having been subjected to a capacitation change effect,
   sperm cells less than 150 minutes after having been subjected to a capacitation change effect,
   sperm cells less than 180 minutes after having been subjected to a capacitation change effect,
   sperm cells less than 210 minutes after having been subjected to a capacitation change effect,
   sperm cells less than 90 minutes after having been subjected to a heparin activation,
   sperm cells less than 120 minutes after having been subjected to a heparin activation,
   sperm cells less than 150 minutes after having been subjected to a heparin activation,
   sperm cells less than 180 minutes after having been subjected to a heparin activation,
   sperm cells less than 210 minutes after having been subjected to a heparin activation,
   and all permutations and combinations of each of the above.
16. A method of separating cells as described in clause 13, or any other clause, wherein said capacitation triggered sperm cells comprise sperm cells in a state of from 180-240 minutes after having been subjected to a heparin activation.
17. A method of separating cells as described in clause 9, or any other clause, and further comprising maintaining said sperm cells in a nurturing environment throughout all said steps.
19. A method of separating cells as described in clause 9, or any other clause, and further comprising the step of including a protein source mixed with said sperm cells throughout all said steps.
19. A method of separating cells as described in clauses 1, 2, 3, 4, 5, 6, 9, or any other clause, and further comprising the step of selecting sperm cells that have never been frozen for said collection of sperm cells.
20. A method of separating cells as described in clauses 1, 2, 3, 4, 5, 6, 9, or any other clause, and further comprising the step of selecting sperm cells that have been frozen and then thawed for said collection of sperm cells.
21. A method of separating cells as described in clauses 1, 2, 3, 4, 5, 6, 9, or any other clause, and further comprising the step of selecting human sperm cells for said collection of sperm cells.
22. A method of separating cells as described in clauses 1, 2, 3, 4, 5, 6, 9, or any other clause, and further comprising the step of selecting non-human sperm cells for said collection of sperm cells.
23. A method of separating cells as described in clause 22, or any other clause, and further comprising the step of selecting bovine sperm cells for said collection of sperm cells.
24. A method of separating cells as described in clauses 22, or any other clause, and further comprising the step of selecting porcine sperm cells for said collection of sperm cells.
25. A method of separating cells as described in clauses 1, 2, 3, 4, 5, 6, 9, or any other clause, and further comprising the step of affirmatively supporting an intrinsically actionable sperm cell sex chromosome related differential effect for said collection of sperm cells.
26. A method of separating cells as described in clause 25, or any other clause, wherein said step of affirmatively supporting an intrinsically actionable sperm cell sex chromosome related differential effect for said collection of sperm cells comprises the step of affirmatively supporting a cellular process differential transition effect for said collection of sperm cells.
27. A method of separating cells as described in clause 25, or any other clause, wherein said step of the step of affirmatively supporting an intrinsically actionable sperm cell sex chromosome related differential effect for said collection of sperm cells comprises the step of pausing said intrinsically actionable sperm cell sex chromosome related differential effect.
28. A method of separating cells as described in clause 25, or any other clause, wherein said step of affirmatively supporting an intrinsically actionable sperm cell sex chromosome related differential effect for said collection of sperm cells comprises a differential effect selected from a group consisting of:
   sialic changes,
   silane surface value effects,
   a cell sialic group effect,
   a cell surface cleaving effect,
   a cell sialic group cleaving effect,
   a cell chemistry effect,
   a cell electrical value,
   a cell electrostatic effect,
   a cell carbohydrate effect,
   a cell surface substance existence,
   a cell surface property,
   a cell pH value,
   a cell ion value,
   a cell membrane effect,
   and all permutations and combinations of each of the above.
29. A method of separating cells as described in clauses 1, 2, 3, 4, 5, 6, 7, 10, or any other clause, and further comprising the step of inducing a differential effect selected from a group consisting of:
   inducing a sperm cell charge differential effect,
   inducing a sperm cell surface charge differential effect,
   inducing a sperm cell chemistry differential effect,
   inducing a sperm cell carbohydrate differential effect,
   inducing a sperm cell sialic group differential effect,
   inducing a polymerase based differential effect,
   inducing a receptor molecule differential effect,
   inducing a Cas9-type differential effect,
   inducing a CRISPR-type differential effect,
   inducing a DNA tag differential effect,
   and all permutations and combinations of each of the above.
30. A method of separating cells as described in clauses 3, 25, or any other clause, and further comprising the step of inducing a sperm cell sex chromosome related differential effect.
31. A method of separating cells as described in clause 30, or any other clause, wherein said step of inducing a sperm cell sex chromosome related differential effect comprises the step of controlled difference inducing a sperm cell sex chromosome related differential effect.
32. A method of separating cells as described in clause 31, or any other clause, wherein said step of controlled difference inducing a sperm cell sex chromosome related differential effect comprises the step of triggering capacitation for said collection of sperm cells.
33. A method of separating cells as described in clause 31, or any other clause, wherein said step of controlled difference inducing a sperm cell sex chromosome related differential effect comprises the step of triggering a differential surface area effect for said collection of sperm cells.
34. A method of separating cells as described in clause 31, or any other clause, wherein said step of controlled difference inducing a sperm cell sex chromosome related differential effect comprises the step of triggering a differential charge effect for said collection of sperm cells.
35. A method of separating cells as described in clauses 1, 30, or any other clause, and further comprising the step of affirmatively establishing said collection of sperm cells in a timed transition state.
36. A method of separating cells as described in clause 35, or any other clause, wherein said step of affirmatively establishing said collection of sperm cells in a timed transition state comprises the step of affirmatively establishing said collection of sperm cells in a frozen then thawed state.
37. A method of separating cells as described in clause 31, or any other clause, wherein said step of controlled difference inducing a sperm cell sex chromosome related differential effect comprises the step of chemically inducing a sperm cell sex chromosome related differential effect.
38. A method of separating cells as described in clause 31, or any other clause, and further comprising the step of subjecting said collection of sperm cells to heparin.
39. A method of separating cells as described in clause 38, or any other clause, wherein said step of subjecting said collection of sperm cells to heparin comprises the step of subjecting said collection of sperm cells to heparin under conditions selected from a group consisting of:

a concentration of about 5 ug heparin per ml of buffer,
a concentration of about 10 ug heparin per ml of buffer,
a concentration of about 15 ug heparin per ml of buffer,
a concentration of about 20 ug heparin per ml of buffer,
a concentration of about 5 ug heparin per ml of buffer for about 120 minutes for sperm cells that have not been previously frozen,
a concentration of about 5 ug heparin per ml of buffer for about 180 minutes for sperm cells that have not been previously frozen,
a concentration of about 5 ug heparin per ml of buffer for from about 180 minutes to about 240 minutes for sperm cells that have not been previously frozen,
a concentration of about 10 ug heparin per ml of buffer for about 120 minutes for sperm cells that have not been previously frozen,
a concentration of about 10 ug heparin per ml of buffer for about 180 minutes for sperm cells that have not been previously frozen,
a concentration of about 10 ug heparin per ml of buffer for from about 180 minutes to about 240 minutes for sperm cells that have not been previously frozen,
a concentration of about 15 ug heparin per ml of buffer for about 120 minutes for sperm cells that have not been previously frozen,
a concentration of about 15 ug heparin per ml of buffer for about 180 minutes for sperm cells that have not been previously frozen,
a concentration of about 15 ug heparin per ml of buffer for from about 180 minutes to about 240 minutes for sperm cells that have not been previously frozen,
a concentration of about 20 ug heparin per ml of buffer for about 120 minutes for sperm cells that have not been previously frozen,
a concentration of about 20 ug heparin per ml of buffer for about 180 minutes for sperm cells that have not been previously frozen,
a concentration of about 20 ug heparin per ml of buffer for from about 180 minutes to about 240 minutes for sperm cells that have not been previously frozen,
a concentration of about 5 ug heparin per ml of buffer for about 30 minutes for thawed previously frozen sperm cells,
a concentration of about 5 ug heparin per ml of buffer for about 60 minutes for thawed previously frozen sperm cells,
a concentration of about 5 ug heparin per ml of buffer for from about 45 minutes to about 60 minutes for thawed previously frozen sperm cells,
a concentration of about 10 ug heparin per ml of buffer for about 30 minutes for thawed previously frozen sperm cells,
a concentration of about 10 ug heparin per ml of buffer for about 60 minutes for thawed previously frozen sperm cells,
a concentration of about 10 ug heparin per ml of buffer for from about 45 minutes to about 60 minutes for thawed previously frozen sperm cells,
a concentration of about 15 ug heparin per ml of buffer for about 30 minutes for thawed previously frozen sperm cells,
a concentration of about 15 ug heparin per ml of buffer for about 60 minutes for thawed previously frozen sperm cells,
a concentration of about 15 ug heparin per ml of buffer for from about 30 minutes to about 60 minutes for thawed previously frozen sperm cells,
a concentration of about 20 ug heparin per ml of buffer for about 30 minutes for thawed previously frozen sperm cells,
a concentration of about 20 ug heparin per ml of buffer for about 60 minutes for thawed previously frozen sperm cells,
a concentration of about 20 ug heparin per ml of buffer for from about 30 minutes to about 60 minutes for thawed previously frozen sperm cells,
until exhibiting an increase of about 0.33 pH,
until exhibiting an increase of about 0.36 pH,
until exhibiting an increase of about 0.39 pH,
until exhibiting an optimal differential effect increase in pH,
until exhibiting an optimal cell viability increase in pH,
and all permutations and combinations of each of the above.
40. A method of separating cells as described in clause 31, or any other clause, and further comprising the step of subjecting said collection of sperm cells to caffeine.
41. A method of separating cells as described in clause 40, or any other clause, wherein said step of subjecting said collection of sperm cells to caffeine comprises the step of subjecting said collection of sperm cells to caffeine selected from a group consisting of:
   until exhibiting an increase of about 0.33 pH,
   until exhibiting an increase of about 0.36 pH,
   until exhibiting an increase of about 0.39 pH,
   until exhibiting an optimal differential effect increase in pH, and
   until exhibiting an optimal cell viability increase in pH.
42. A method of separating cells as described in clause 10, or any other clause, and further comprising the step of subjecting said collection of sperm cells to a pH altering buffer.
43. A method of separating cells as described in clause 42, or any other clause, wherein said step of subjecting said collection of sperm cells to a pH altering buffer comprises the step of subjecting said collection of sperm cells to a pH altering buffer selected from a group consisting of:
   subjecting said collection of sperm cells to a buffer having a pH that increases the environment of said collection of sperm cells by about 0.33 pH,
   subjecting said collection of sperm cells to a buffer having a pH that increases the environment of said collection of sperm cells by about 0.36 pH, and
   subjecting said collection of sperm cells to a buffer having a pH that increases the environment of said collection of sperm cells by about 0.39 pH.
44. A method of separating cells as described in clause 10, or any other clause, wherein said step of controlled difference inducing a sperm cell sex chromosome related differential effect comprises the step of inducing a sperm cell charge differential effect.
45. A method of separating cells as described in clause 44, or any other clause, wherein said step of inducing a sperm cell charge differential effect comprises the step of inducing a sperm cell surface charge differential effect.
46. A method of separating cells as described in clause 45, or any other clause, wherein said step of step of inducing a sperm cell surface charge differential effect comprises the step of inducing a sperm cell zeta charge differential effect.
47. A method of separating cells as described in clause 46, or any other clause, wherein said step of inducing a sperm cell zeta charge differential effect comprises the step of inducing a sperm cell zeta charge differential effect selected from a group consisting of:
   inducing a sperm cell substantially uncharged differential effect, and
   inducing a sperm cell low charge differential effect.
48. A method of separating cells as described in clause 10, or any other clause, and further comprising the step of subjecting said collection of sperm cells to a differential change inducing buffer.
49. A method of separating cells as described in clause 48, or any other clause, wherein said step of subjecting said collection of sperm cells to a differential change inducing buffer comprises the step of subjecting said collection of sperm cells to a buffer containing an operative amount of a salt.
50. A method of separating cells as described in clause 48, or any other clause, wherein said step of subjecting said collection of sperm cells to a differential change inducing buffer comprises the step of subjecting said collection of sperm cells to a more basic buffer.
51. A method of separating cells as described in clause 10, or any other clause, and further comprising the step of inducing a sperm cell chemistry differential effect.
52. A method of separating cells as described in clause 51, or any other clause, wherein said step of inducing a sperm cell chemistry differential effect comprises the step of inducing a sperm cell carbohydrate differential effect.
53. A method of separating cells as described in clause 51, or any other clause, wherein said step of inducing a sperm cell chemistry differential effect comprises the step of inducing a sperm cell sialic group differential effect.
54. A method of separating cells as described in clause 52, or any other clause, wherein said step of inducing a sperm cell sialic group differential effect comprises the step of inducing a sperm cell sialic group cleaving differential effect.
55. A method of separating cells as described in clause 26, or any other clause, and further comprising the steps of:
   determining a usable level of sperm cell sex chromosome related differential effect, and
   affirmatively effecting said level of sperm cell sex chromosome related differential effect for said collection of sperm cells.
56. A method of separating cells as described in clause 55, or any other clause, wherein said step of determining a usable level of sperm cell sex chromosome related differential effect comprises the step of determining a maximum difference level of sperm cell sex chromosome related differential effect.
57. A method of separating cells as described in clause 55, or any other clause, wherein said step of determining a usable level of sperm cell sex chromosome related differential effect comprises the step of determining a usable pH indicated level of sperm cell sex chromosome related differential effect.
58. A method of separating cells as described in clause 57, or any other clause, wherein said step of determining a usable pH indicated level of sperm cell sex chromosome related differential effect comprises the step of determining a usable pH indicated level of sperm cell sex chromosome related differential effect selected from a group consisting of:
   determining a pH increase for the environment of said collection of sperm cells of about 0.33 pH,
   determining a pH increase for the environment of said collection of sperm cells of about 0.36 pH, and
   determining a pH increase for the environment of said collection of sperm cells of about 0.39 pH.
59. A method of separating cells as described in clause 55, or any other clause, wherein said step of determining a usable level of sperm cell sex chromosome related differential effect comprises the step of timing a cellular process differential transition effect for said collection of sperm cells.
60. A method of separating cells as described in clause 59, or any other clause, wherein said step of timing a cellular process differential transition effect for said collection of sperm cells comprises the step of timing a cellular process differential transition effect for said collection of sperm cells selected from a group consisting of:
   terminating said cellular process differential transition effect at about 90 minutes,
   terminating said cellular process differential transition effect at about 120 minutes,
   terminating said cellular process differential transition effect at about 150 minutes, and
   terminating said cellular process differential transition effect at up to about 150 minutes.
61. A method of separating cells as described in clause 13, or any other clause, and further comprising the step of utilizing said sperm cells after having experienced said cellular process differential transition effect for between about 180 to 240 minutes.
62. A method of separating cells as described in clause 59, or any other clause, wherein said step of timing a cellular process differential transition effect for said collection of sperm cells comprises the step of timing a cellular process differential transition effect for said collections of sperm cells selected from a group consisting of:
   utilizing frozen-thawed sperm cells after having been thawed for about 4 hours,
   utilizing frozen-thawed sperm cells after having been thawed for about 6 hours,
   utilizing frozen-thawed sperm cells after having been thawed for about 8 hours,
   utilizing frozen-thawed sperm cells after having been thawed for about 12 hours, and
   utilizing frozen-thawed sperm cells after having been thawed for overnight.
63. A method of separating cells as described in clause 10, or any other clause, and further comprising the step of incubating said collection of sperm cells with associatable particles.
64. A method of separating cells as described in clauses 1, 2, 3, 4, 5, 6, 9, or any other clause, wherein said collection of sperm cells initially exist in seminal plasma, and further comprising the step of removing said seminal plasma from said collection of sperm cells.
65. A method of separating cells as described in clause 64, or any other clause, and further comprising the step of resubjecting said collection of sperm cells to a seminal plasma mixture
66. A method of separating cells as described in clause 64, or any other clause, and further comprising the step of resubjecting said collection of sperm cells to a mixture containing substances selected from a group consisting of:
   seminal plasma, and
   BSA.
67. A method of separating cells as described in clause 10, or any other clause, and further comprising the step of quenching said induced sperm cell sex chromosome related differential effect.
68. A method of separating cells as described in clause 67, or any other clause, and further comprising the step of quenching capacitation of said collection of sperm cells.
69. A method of separating cells as described in clause 68, or any other clause, wherein said step of quenching capacitation of said collection of sperm cells comprises the step of subjecting said collection of sperm cells to a mixture containing substances selected from a group consisting of:
   seminal plasma, and
   BSA.
70. A method of separating cells as described in clauses 1, 2, 3, 4, 5, 6, or any other clause, and further comprising the step of suspending sperm cell associatable particles with said collection of sperm cells to create a suspension of sperm cell associatable particles in said collection of sperm cells.
71. A method of separating cells as described in clauses 1, 2, 3, 4, 5, 6, 7, or any other clause, and further comprising the step of combining sperm cell associatable particles with said collection of sperm cells to create an unsuspended collection of sperm cell associatable particles in said collection of sperm cells.
72. A method of separating cells as described in clauses 1, 2, 3, 4, 5, 6, 7, or any other clause, and further comprising the step of mixing sperm cell associatable particles with said collection of sperm cells to create a mixture of sperm cell associatable particles in said collection of sperm cells.
73. A method of separating cells as described in clauses 1, 2, 3, 4, 5, 6, 7, or any other clause, and further comprising the step of moving said sperm cell associatable particles through said collection of sperm cells.
74. A method of separating cells as described in clauses 1, 2, 3, 4, 5, 6, 7, or any other clause, and further comprising the step of moving said collection of sperm cells through said sperm cell associatable particles.
75. A method of separating cells as described in clauses 1, 2, 3, 4, 5, 6, 7, or any other clause, and further comprising the step of sperm cell associatable particles having a mean diameter at least about 1000 nm.
76. A method of separating cells as described in clauses 3, 5, 6, or any other clause, and further comprising the step of associating a desired portion of said collection of sperm cells with a substance.
77. A method of separating cells as described in clause 7, 76, or any other clause, wherein said step of associating comprises the step of differentially associating a specific chromosome bearing type of sperm cell with said sperm cell associatable particles.
78. A method of separating cells as described in clauses 1, 2, 4, 7, 76, or any other clause, wherein said step of associating comprises the step of electrostatically associating a desired portion of said collection of sperm cells with said sperm cell associatable particles.
79. A method of separating cells as described in clauses 1, 2, 4, 7, 76, or any other clause, wherein said step of associating comprises the step of chemically associating a desired portion of said collection of sperm cells with said sperm cell associatable particles.
80. A method of separating cells as described in clauses 78, 79, or any other clause, wherein said sperm cell associatable particles comprise coated sperm cell associatable particles.
81. A method of separating cells as described in clause 80, or any other clause, wherein said coated sperm cell associatable particles comprise coated sperm cell associatable particles selected from a group consisting of:
   carboxyl modified silane coated sperm cell associatable particles,
   carbohydrate coated sperm cell associatable particles,
   ligand coated sperm cell associatable particles,
   Sambucus nigra agglutinin (SNA) coated sperm cell associatable particles,
   Monosaccharide coated sperm cell associatable particles,
   antibody coated sperm cell associatable particles,
   sperm cell differentiatable condition active sperm cell associatable particles,
   polymerase associatable particles,
   receptor molecule associatable particles,
   Cas9-type associatable particles,
   CRISPR-type associatable particles,
   DNA tag associatable particles,
   and all permutations and combinations of each of the above.
82. A method of separating cells as described in clauses 1, 2, 3, 6, or any other clause, and further comprising the step of separating through action of sperm cell associatable particles.
83. A method of separating cells as described in clause 7, 8, 29, or any other clause, and further comprising cell associatable particles selected from a group consisting of:
   carboxyl modified silane coated sperm cell associatable particles,
   carbohydrate coated sperm cell associatable particles,
   ligand coated sperm cell associatable particles,
   Sambucus nigra agglutinin (SNA) coated sperm cell associatable particles,
   Monosaccharide coated sperm cell associatable particles,
   antibody coated sperm cell associatable particles,
   sperm cell differentiatable condition active sperm cell associatable particles,
   polymerase associatable particles,
   receptor molecule associatable particles,
   Cas9-type associatable particles,
   CRISPR-type associatable particles,
   DNA tag associatable particles,
   and all permutations and combinations of each of the above.
84. A method of separating cells as described in clauses 2, 3, 4, 5, 6, or any other clause, and further comprising the steps of combining sperm cell associatable particles with said collection of sperm cells to establish a fluid combination of sperm cell associatable particles and sperm cells.
85. A method of separating cells as described in clauses 1, 7, 83, 84, or any other clause, and further comprising the step of combining cell associatable nanoparticles with said collection of cells to establish a fluid combination of cell associatable nanoparticles and cells.
86. A method of separating cells as described in clauses 1, 7, 83, 84 or any other clause, and further comprising the step of combining cell associatable particles selected from a group consisting of:
   cell associatable nanoparticles,
   cell associatable particles having a mean diameter of between 10 nm and 999 nm,
   cell associatable microparticles,
   cell associatable particles having a mean diameter of between 100 nm and 100 um,
   cell associatable particles having a mean diameter of not more than about 1000 nm,
   cell associatable particles having a mean diameter of about 670 nm,
   cell associatable particles having a mean diameter at least about 100 nm,
   cell associatable particles having a mean diameter at least about 300 nm,
   cell associatable particles having a mean diameter at least about 500 nm, and
   cell associatable particles having a mean diameter at least about 600 nm.
87. A method of separating cells as described in clause 80, 83, or any other clause, wherein said step of separating through action of said cell associatable particles comprises the step of separating through action of said cell associatable particles selected from a group consisting of:
   iron oxide particles,
   glass particles,
   silica particles,
   silica with aluminum substitution particles,
   borosilicate particles,
   plastic particles,
   PVP particles,
   polyvinlypropylene particles,
   polyvinylpyrrolidone particles,
   polystyrene particles,
   melamine particles,
   PMMA particles,
   polylactide particles,
   particles bound to polar molecules,
   dextran particles,
   functionalized surface particles,
   and all permutations and combinations of each of the above.
88. A method of separating cells as described in clause 85, or any other clause, wherein said step of step of combining cell associatable nanoparticles with said collection of cells to establish a fluid combination of cell associatable nanoparticles and cells comprises the step of combining cell associatable nanoparticles with said collection of cells to establish a fluid combination of cell associatable nanoparticles and cells selected from a group consisting of:
   combining particles stabilized by pH adjustment with said collection of cells to establish a fluid combination of sperm cell associatable particles and cells,
   combining a pH particle combination stabilized particle containing fluid with said collection of cells to establish a fluid combination of cell associatable particles and cells,
   combining a particle concentrated particle fluid with said collection of cells to establish a fluid combination of cell associatable particles and cells,
   combining a stable particle fluid with said collection of cells to establish a fluid combination of cell associatable particles and cells,
   combining a stable concentration level particle fluid with said collection of cells to establish a fluid combination of cell associatable particles and cells,
   combining a particle size-concentration level coordinated stable particle fluid with said collection of cells to establish a fluid combination of cell associatable particles and cells,
   combining a 50 nm particle size-50% wt solids particle fluid with said collection of cells to establish a fluid combination of cell associatable particles and cells,
   combining a 10 nm particle size-30% wt solids particle fluid with said collection of cells to establish a fluid combination of cell associatable particles and cells,
   and all permutations and combinations of each of the above.
89. A method of separating cells as described in clauses 1, 7, 83, 84, or any other clause, wherein said step of separating comprises the step of magnetically separating at least some of said cells.
90. A method of separating cells as described in clauses 1, 7, 83, 84, or any other clause, wherein said step of separating comprises the step of electrostatically separating at least some of said cells.
91. A method of separating cells as described in clauses 1, 7, 83, 84, or any other clause, wherein said step of separating comprises the step of separating at least some of said cells by electrophoresis.
92. A method of separating cells as described in clauses 1, 7, 83, 84 or any other clause, wherein said step of separating comprises the step of gravimetrically separating at least some of said cells.
93. A method of separating cells as described in clause 92, or any other clause, wherein said step of gravimetrically separating at least some of said cells comprises the step of utilizing cell associatable microparticles.
94. A method of separating cells as described in clause 93, or any other clause, wherein said wherein said step of gravimetrically separating at least some of said cells comprises the step of utilizing cell associatable particles having a mean diameter at least about 1000 nm.
95. A method of separating cells as described in clause 92, or any other clause, wherein said step of gravimetrically separating at least some of said cells comprises the step of gravimetrically separating with a gravimetric force greater than any cell motility force.
96. A method of separating cells as described in clause 92, or any other clause, wherein said step of gravimetrically separating with a gravimetric force greater than any cell motility force comprises the step of separating at least some of said cells by centrifugation.
97. A method of separating cells as described in clauses 1, 2, 3, 4, 5, 7, or any other clause, wherein said step of separating comprises the step of bulk separating said cells.
98. A method of separating cells as described in clauses 6, 8, 29, 97, or any other clause, wherein said step of bulk separating comprises the step of bulk separating cells while still in said collection of cells.
99. A method of separating cells as described in clauses 6, 8, 29, 97, or any other clause, wherein said step of bulk separating comprises the step of bulk separating selected from a group consisting of:
   separating said cells more than one-at-a-time,
   simultaneously separating a significant quantity of said cells in said collection of cells,
   simultaneously separating the majority of the desired type of said cells in said collection of cells,
   simultaneously separating substantially all of the desired type of said cells in said collection of cells, and
   simultaneously separating at least ten thousand of said cells at a time.
100. A method of separating cells as described in clauses 1, 2, 3, 4, 6, 7, or any other clause, and further comprising the step of selectively impacting substantially only one type of cells from said collection of cells while leaving the other type of cells from said collection of cells substantially unimpacted.
101. A method of separating cells as described in clauses 5, 100, or any other clause, wherein said step of selectively impacting comprises the step of causing no substantial effect on one type of cells from said collection of cells.
102. A method of separating cells as described in clause 101, or any other clause, wherein said step of causing no substantial effect on one type of cells from said collection of cells comprises the step of subjecting one type of cells from said collection of cells to only natural effects.
103. A method of separating cells as described in clause 101, or any other clause, wherein said step of causing no substantial effect on one type of cells from said collection of cells comprises the step of causing no substantially effect on one type of cells from said collection of cells selected from a group consisting of:
   avoiding associating any unnatural substance with one type of cells from said collection of cells,
   avoiding staining one type of cells from said collection of cells,
   avoiding subjecting one type of cells from said collection of cells to any non-naturally occurring external forces,
   avoiding hyperactivity in one type of cells from said collection of cells,
   avoiding any physical activity for one type of cells from said collection of cells,
   and all permutations and combinations of each of the above.
104. A method of separating cells as described in clause 9, or any other clause, wherein said step of separating comprises the step of separating at least some X-bearing sperm cells and Y-bearing sperm cells.
105. A method of separating cells as described in clauses 1, 2, 3, 4, 5, 6, 101, or any other clause, and further comprising the step of separating at least some of one type of cells from said collection of cells while acting substantially passively with respect to another type of said cells.
106. A method of separating cells as described in clause 105, or any other clause, wherein said step of separating at least some of one type of cells from said collection of cells while acting substantially passively with respect to another type of said cells comprises the step of non-cell motility separating at least some of one type of cells from said collection of cells.
107. A method of separating cells as described in clause 106, or any other clause, wherein said step of separating at least some of one type of cells from said collection of cells while acting substantially passively with respect to another type of said cells comprises the step of external force separating at least some of one type of cells from said collection of cells.
108. A method of separating cells as described in clause 107, or any other clause, wherein said step of external force separating at least some of one type of cells from said collection of cells comprises the step of external force separating at least some of one type of cells from said collection of cells selected from a group consisting of:
   magnetically separating at least some of one type of cells from said collection of cells,
   electrostatically separating at least some of one type of cells from said collection of cells,
   separating at least some of one type of cells from said collection of cells by electrophoresis,
   gravimetrically separating at least some of one type of cells from said collection of cells, and
   separating at least some of one type of cells from said collection of cells by centrifugation.
109. A method of separating cells as described in clauses 1, 2, 3, 4, 5, 6, 7, or any other clause, wherein said step of separating comprises the step of separating selected from a group consisting of:
   separating substantially all of a desired type of said cells in said collection of cells,
   separating at least about 70% of a desired type of said cells in said collection of cells,
   separating at least about 80% of a desired type of said cells in said collection of cells,
   separating at least about 90% of a desired type of said cells in said collection of cells,
   separating at least about 95% of a desired type of said cells in said collection of cells,
   separating at least about 97% of a desired type of said cells in said collection of cells,
   separating at least about 98% of a desired type of said cells in said collection of cells,
   separating at least about 99% of a desired type of said cells in said collection of cells, and
   separating so as to leave no appreciable viable cells of a desired type of said cells in said collection of cells.
110. A method of separating cells as described in clauses 1, 2, 3, 4, 5, 6, 7, or any other clause, wherein said step of separating comprises the step of capturing a desired type of said cells in said collection of cells.
111. A method of separating cells as described in clauses 1, 2, 3, 4, 5, 6, 7, or any other clause, wherein said step of separating comprises the step of gravimetrically separating at least some of one type of cells from said collection of cells.
112. A method of separating cells as described in clauses 7, 111, or any other clause, wherein said step of gravimetrically separating comprises the step of gravitationally separating at least some of one type of cells from said collection of cells.
113. A method of separating cells as described in clauses 7, 111, or any other clause, wherein said step of gravimetrically separating comprises the step of enhanced force separating at least some of one type of cells from said collection of cells.
114. A method of separating cells as described in clauses 7, 111, or any other clause, wherein said step of gravimetrically separating comprises the step of centrifugationally separating at least some of one type of cells from said collection of cells.
115. A method of separating cells as described in clause 7, or any other clause, wherein said cells comprise sperm cells.
116. A method of separating cells as described in clause 115, or any other clause, wherein said sperm cells comprise X-bearing sperm cells and Y-bearing sperm cells.
117. A method of separating cells as described in clause 116, or any other clause, wherein said X-bearing sperm cells and Y-bearing sperm cells comprise X-bearing sperm cells and Y-bearing sperm cells for artificial insemination use.
118. A method of separating cells as described in clause 115, or any other clause, wherein said sperm cells comprise dying or functionally impaired sperm cells.
119. A method of separating cells as described in clause 7, or any other clause, wherein said cells comprise dying or functionally impaired cells.
120. A system for separation of X-bearing sperm and Y-bearing sperm cells comprising:
   a collection of sex chromosome differentially exhibiting sperm cells with both X-bearing sperm cells and Y-bearing sperm cells in said collection;
   a plurality of sperm cell sex chromosome differentially associatable particles fluidically combined with said collection of sex chromosome differential exhibiting sperm cells so as to establish a fluid combination of sperm cell sex chromosome differentially associatable particles and sex chromosome differentially exhibiting sperm cells; and
   a particle separation modality to which said sperm cell sex chromosome differentially associatable particles in said fluid combination of sperm cell sex chromosome differentially associatable particles and sex chromosome differentially exhibiting sperm cells are responsive.
121. A system for separation of X-bearing sperm and Y-bearing sperm cells comprising:
   a collection of sex chromosome differentially exhibiting sperm cells with both X-bearing sperm cells and Y-bearing sperm cells in said collection;
   a sperm cell sex chromosome differentially associatable substance situated proximate to said collection of sex chromosome differentially exhibiting sperm cells; and
   a sperm cell separation modality to which at least some of said sex chromosome differentially exhibiting sperm cells are responsive.
122. A system for separation of X-bearing sperm and Y-bearing sperm cells comprising:
   a collection of sex chromosome differentially exhibiting sperm cells with both X-bearing sperm cells and Y-bearing sperm cells in said collection;
   a sialic acid group differentially associated substance; and
   a sperm cell separation modality to which at least some of said sex chromosome differentially exhibiting sperm cells are responsive.
123. A system for bulk separation of X-bearing sperm and Y-bearing sperm cells comprising:
   a collection of sex chromosome differentially exhibiting sperm cells with both X-bearing sperm cells and Y-bearing sperm cells in said collection; and
   a bulk sperm cell separation modality to which at least some of said sex chromosome differentially exhibiting sperm cells are responsive.
124. A system for separation of cells comprising:
   a collection of differentially exhibiting cells;
   a plurality of cell differentially associatable particles fluidically combined with said collection of cells so as to establish a fluid combination of cell differentially associatable particles and differentially exhibiting cells; and
   a gravimetric separation modality to which said cell differentially associatable particles in said fluid combination of cell differentially associatable particles and differentially exhibiting cells are responsive.
125. A system for bulk separation of cells comprising:
   a differential effect inducer to which cells are responsive;
   a collection of differentially exhibiting cells; and
   a bulk cell separation modality to which at least some of said differentially exhibiting cells are responsive.
126. A system for separation of cells as described in clauses 120, 121, 122, 123, 124, or any other clause, wherein both said X-bearing and Y-bearing sperm cells in said collection prior to separation comprise functionally viable X-bearing and Y-bearing sperm cell.
127. A system for separation of cells as described in clause 126, or any other clause, wherein said functionally viable X-bearing and Y-bearing sperm cells comprise functionally viable cells usable in practical application for fertilization processes.
128. A system for separation of cells as described in clause 126, or any other clause, wherein said functionally viable cells usable in practical application for fertilization processes comprise functionally viable cells usable in practical application for artificial insemination processes.
129. A system for separation of cells as described in clause 126, or any other clause, wherein both said X-bearing and Y-bearing sperm cells in said collection prior to separation comprise sperm cells in a state where they are practically usable for fertilization without substantial loss of such cells selected from a group consisting of:
   after overnight storage,
   after shipping in a natural state,
   after freezing, shipping and thawing,
   after having been frozen,
   after having been frozen and then thawed,
   after at least about 8 hours for cells held in an unfrozen state in seminal plasma,
   after at least about 16 hours for cells held in an unfrozen state in seminal plasma,
   after at least about 24 hours for cells held in an unfrozen state in seminal plasma,
   after at least about 30 minutes after thawing for cells frozen and then thawed,
   after at least about 45 minutes after thawing for cells frozen and then thawed,
   after at least about 1 hour after thawing for cells frozen and then thawed,
   after at least about 2 hours after thawing for cells frozen and then thawed,
   cells that remain practically usable for fertilization without a loss of more than 20% of such cells,
   cells that remain practically usable for fertilization without a loss of more than 30% of such cells,
   cells that remain practically usable for fertilization without a loss of more than 40% of such cells,
   and all permutations and combinations of each of the above.
130. A system for separation of cells as described in clause 126, or any other clause, wherein said functionally viable X-bearing and Y-bearing sperm cells comprise capacitation triggered sperm cells.
131. A system for separation of cells as described in clause 130, or any other clause, wherein said capacitation triggered sperm cells comprise pre-acrosome reaction initiation sperm cells.
132. A system for separation of cells as described in clause 130, or any other clause, wherein said capacitation triggered sperm cells comprise sperm cells selected from a group consisting of:
   sperm cells less than 90 minutes after having been subjected to a capacitation change effect,
   sperm cells less than 120 minutes after having been subjected to a capacitation change effect,
   sperm cells less than 150 minutes after having been subjected to a capacitation change effect,
   sperm cells less than 180 minutes after having been subjected to a capacitation change effect,
   sperm cells less than 210 minutes after having been subjected to a capacitation change effect,
   sperm cells less than 90 minutes after having been subjected to a heparin activation,
   sperm cells less than 120 minutes after having been subjected to a heparin activation,
   sperm cells less than 150 minutes after having been subjected to a heparin activation,
   sperm cells less than 180 minutes after having been subjected to a heparin activation,
   sperm cells less than 210 minutes after having been subjected to a heparin activation, and
   and all permutations and combinations of each of the above.
133. A system for separation of cells as described in clause 130, or any other clause, wherein said capacitation triggered sperm cells comprise sperm cells in a state of from 180-240 minutes after having been subjected to a heparin activation.
134. A system for separation of cells as described in clause 126, or any other clause, and further comprising one or more nurturing environments for said cells that exist in all aspects of the system.
135. A system for separation of cells as described in clause 126, or any other clause, and further comprising one or more protein sources for said cells that exist in all aspects of the system.
136. A system for separation of cells as described in clauses 120, 121, 122, 123, 126, or any other clause, and further comprising sperm cells that have never been frozen for said collection of sperm cells.
137. A system for separation of cells as described in clauses 120, 121, 122, 123, 126, or any other clause, and further comprising sperm cells that have been frozen and thawed for said collection of sperm cells.
138. A system for separation of cells as described in clauses 120, 121, 122, 123, 126, or any other clause, and further comprising human sperm cells.
139. A system for separation of cells as described in clauses 120, 121, 122, 123, 126, or any other clause, and further comprising non-human sperm cells.
140. A system for separation of cells as described in clause 139, or any other clause, and further comprising bovine sperm cells.
141. A system for separation of cells as described in clause 139, or any other clause, and further comprising porcine sperm cells.
142. A system for separation of cells as described in clauses 120, 121, 122, 123, 124, 126, or any other clause, and further comprising a differential effect affirmative support.
143. A system for separation of cells as described in clause 142, or any other clause, wherein said differential effect affirmative support comprises a cellular process differential effect affirmative support.
144. A system for separation of cells as described in clause 142, or any other clause, wherein said differential effect affirmative support comprises a differential effect pause element.
145. A system for separation of cells as described in clause 142, or any other clause, wherein said differential effect affirmative support comprises a differential effect selected from a group consisting of:
   sialic changes,
   silane surface value effects,
   a cell sialic group effect,
   a cell surface cleaving effect,
   a cell sialic group cleaving effect,
   a cell chemistry effect,
   a cell electrical value,
   a cell electrostatic effect,
   a cell carbohydrate effect,
   a cell surface substance existence,
   a cell surface property,
   a cell pH value,
   a cell ion value,
   a cell membrane effect,
   and all permutations and combinations of each of the above.
146. A system for separation of cells as described in clauses 120, 121, 122, 123, 124, 125, and further comprising a differential effect inducer selected from a group consisting of:
   a sperm cell chemistry differential effect inducer,
   a sperm cell carbohydrate differential effect inducer,
   a sperm cell sialic group differential effect inducer,
   a polymerase-based inducer,
   a receptor molecule inducer,
   a Cas9-type inducer,
   a CRISPR-type inducer,
   a DNA tag inducer,
   and all permutations and combinations of each of the above.
147. A system for separation of cells as described in clause 142, or any other clause, and further comprising a differential effect inducer.
148. A system for separation of cells as described in clause 147, or any other clause, wherein said differential effect inducer comprises a differential effect difference control.
149. A system for separation of cells as described in clause 148, or any other clause, wherein said differential effect difference control comprises a capacitation trigger.
150. A system for separation of cells as described in clause 148, or any other clause, wherein said differential effect difference control comprises a differential surface area effect trigger.
151. A system for separation of cells as described in clause 148, or any other clause, wherein said differential effect difference control comprises a differential charge effect trigger.
152. A system for separation of cells as described in clauses 120, 147, or any other clause, and further comprising a transition state timer.
153. A system for separation of cells as described in clause 152, or any other clause, wherein said transition state timer comprises affirmatively frozen then thawed sperm cells.
154. A system for separation of cells as described in clause 148, or any other clause, wherein said differential effect difference control comprises a differential effect chemical inducer agent.
155. A system for separation of cells as described in clause 148, or any other clause, and further comprising a collection of sperm cells that have been subjected to heparin.
156. A system for separation of cells as described in clause 155, or any other clause, wherein said collection of sperm cells that have been subjected to heparin comprises a collection of sperm cells that have been subjected to heparin selected from a group consisting of:
   a concentration of about 5 ug heparin per ml of buffer,
   a concentration of about 10 ug heparin per ml of buffer,
   a concentration of about 15 ug heparin per ml of buffer,
   a concentration of about 20 ug heparin per ml of buffer,
   a concentration of about 5 ug heparin per ml of buffer for about 120 minutes for sperm cells that have not been previously frozen,
   a concentration of about 5 ug heparin per ml of buffer for about 180 minutes for sperm cells that have not been previously frozen,
   a concentration of about 5 ug heparin per ml of buffer for from about 180 minutes to about 240 minutes for sperm cells that have not been previously frozen,
   a concentration of about 10 ug heparin per ml of buffer for about 120 minutes for sperm cells that have not been previously frozen,
   a concentration of about 10 ug heparin per ml of buffer for about 180 minutes for sperm cells that have not been previously frozen,
   a concentration of about 10 ug heparin per ml of buffer for from about 180 minutes to about 240 minutes for sperm cells that have not been previously frozen,
   a concentration of about 15 ug heparin per ml of buffer for about 120 minutes for sperm cells that have not been previously frozen,
   a concentration of about 15 ug heparin per ml of buffer for about 180 minutes for sperm cells that have not been previously frozen,
   a concentration of about 15 ug heparin per ml of buffer for from about 180 minutes to about 240 minutes for sperm cells that have not been previously frozen,
   a concentration of about 20 ug heparin per ml of buffer for about 120 minutes for sperm cells that have not been previously frozen,
   a concentration of about 20 ug heparin per ml of buffer for about 180 minutes for sperm cells that have not been previously frozen,
   a concentration of about 20 ug heparin per ml of buffer for from about 180 minutes to about 240 minutes for sperm cells that have not been previously frozen,
   a concentration of about 5 ug heparin per ml of buffer for about 30 minutes for thawed previously frozen sperm cells,
   a concentration of about 5 ug heparin per ml of buffer for about 60 minutes for thawed previously frozen sperm cells,
   a concentration of about 5 ug heparin per ml of buffer for from about 45 minutes to about 60 minutes for thawed previously frozen sperm cells,
   a concentration of about 10 ug heparin per ml of buffer for about 30 minutes for thawed previously frozen sperm cells,
   a concentration of about 10 ug heparin per ml of buffer for about 60 minutes for thawed previously frozen sperm cells,
   a concentration of about 10 ug heparin per ml of buffer for from about 45 minutes to about 60 minutes for thawed previously frozen sperm cells,
   a concentration of about 15 ug heparin per ml of buffer for about 30 minutes for thawed previously frozen sperm cells,
   a concentration of about 15 ug heparin per ml of buffer for about 60 minutes for thawed previously frozen sperm cells,
   a concentration of about 15 ug heparin per ml of buffer for from about 30 minutes to about 60 minutes for thawed previously frozen sperm cells,
   a concentration of about 20 ug heparin per ml of buffer for about 30 minutes for thawed previously frozen sperm cells,
   a concentration of about 20 ug heparin per ml of buffer for about 60 minutes for thawed previously frozen sperm cells,
   a concentration of about 20 ug heparin per ml of buffer for from about 30 minutes to about 60 minutes for thawed previously frozen sperm cells,
   until exhibiting an increase of about 0.33 pH,
   until exhibiting an increase of about 0.36 pH,
   until exhibiting an increase of about 0.39 pH,
   until exhibiting an optimal differential effect increase in pH,
   until exhibiting an optimal cell viability increase in pH,
   and all permutations and combinations of each of the above.
157. A system for separation of cells as described in clause 148, or any other clause, and further comprising a collection of sperm cells that have been subjected to caffeine
158. A system for separation of cells as described in clause 157, or any other clause, wherein said caffeine comprises a collection of sperm cells that have been subjected to caffeine under conditions selected from a group consisting of:
   until exhibiting an increase of about 0.33 pH,
   until exhibiting an increase of about 0.36 pH,
   until exhibiting an increase of about 0.39 pH,
   until exhibiting an optimal differential effect increase in pH, and
   until exhibiting an optimal cell viability increase in pH.
159. A system for separation of cells as described in clause 148, or any other clause, and further comprising collection of sperm cells that have been subjected a pH altering buffer.
160. A system for separation of cells as described in clause 159, or any other clause, wherein said collection of sperm cells that have been subjected a pH altering buffer comprises a collection of sperm cells that have been subjected a pH altering buffer selected from a group consisting of:
   a buffer having a pH that increases the environment of said collection of sperm cells by about 0.33 pH,
   a buffer having a pH that increases the environment of said collection of sperm cells by about 0.36 pH, and
   a buffer having a pH that increases the environment of said collection of sperm cells by about 0.39 pH.
161. A system for separation of cells as described in clause 148, or any other clause, wherein said a differential effect difference control comprises a sperm cell charge differential inducer.
162. A system for separation of cells as described in clause 161, or any other clause, wherein said sperm cell charge differential inducer comprises a sperm cell surface charge differential inducer.
163. A system for separation of cells as described in clause 162, or any other clause, wherein said sperm cell charge differential inducer comprises a sperm cell zeta charge differential inducer.
164. A system for separation of cells as described in clause 163, or any other clause, wherein said sperm cell zeta charge differential inducer comprises a sperm cell zeta charge differential inducer selected from a group consisting of:
   a sperm cell substantially uncharged differential inducer, and
   a sperm cell low charge differential inducer.
165. A system for separation of cells as described in clause 148, or any other clause, and further comprising a sperm cell differential change inducing buffer.
166. A system for separation of cells as described in clause 165, or any other clause, wherein said sperm cell differential change inducing buffer comprises a sperm cell buffer containing an operative amount of salt.
167. A system for separation of cells as described in clause 165, or any other clause, wherein said a sperm cell differential change inducing buffer comprises a sperm cell more basic buffer.
168. A system for separation of cells as described in clause 148, or any other clause, and further comprising a sperm cell chemistry differential effect inducer.
169. A system for separation of cells as described in clause 168, or any other clause, wherein said sperm cell chemistry differential effect inducer comprises a sperm cell carbohydrate differential effect inducer.
170. A system for separation of cells as described in clause 168, or any other clause, wherein said sperm cell chemistry differential effect inducer comprises a sperm cell sialic group differential effect inducer.
171. A system for separation of cells as described in clause 170, or any other clause, wherein said sperm cell sialic group differential effect inducer comprises a sperm cell sialic group cleaving inducer.
172. A system for separation of cells as described in clause 142, or any other clause, and further comprising a sperm cell sex chromosome differential effect usable level indicator.
173. A system for separation of cells as described in clause 72, or any other clause, wherein said a sperm cell sex chromosome differential effect usable level indicator comprises a sperm cell sex chromosome maximum differential effect difference level indicator.
174. A system for separation of cells as described in clause 72, or any other clause, wherein said a sperm cell sex chromosome differential effect usable level indicator comprises a sperm cell sex chromosome differential effect pH indicator.
175. A system for separation of cells as described in clause 174, or any other clause, wherein said sperm cell sex chromosome differential effect pH indicator comprises a sperm cell sex chromosome differential effect pH indicator selected from the group consisting of:
   a sperm cell sex chromosome differential effect of about 0.33 pH increase indicator,
   a sperm cell sex chromosome differential effect of about 0.36 pH increase indicator, and
   a sperm cell sex chromosome differential effect of about 0.39 pH increase indicator.
176. A system for separation of cells as described in clause 172, or any other clause, wherein said sperm cell sex chromosome differential effect usable level indicator comprises a cellular process differential transition effect timer.
177. A system for separation of cells as described in clause 176, or any other clause, wherein said a cellular process differential transition effect timer comprises a cellular process differential transition effect timer selected from a group consisting of:
   a cellular process 90-minute differential transition effect timer,
   a cellular process 120-minute differential transition effect timer,
   a cellular process 150-minute differential transition effect timer, and
   a cellular process at up to 150-minute differential transition effect timer.
178. A system for separation of cells as described in clause 130, or any other clause, and further comprising a cellular process 180 to 240-minute differential transition effect timer.
179. A system for separation of cells as described in clause 176, or any other clause, wherein said cellular process differential transition effect timer comprises a cellular process differential transition effect timer selected from a group consisting of:
   sperm cells that have been frozen and thawed for about 4 hours for said collection of sperm cells,
   sperm cells that have been frozen and thawed for about 6 hours for said collection of sperm cells,
   sperm cells that have been frozen and thawed for about 8 hours for said collection of sperm cells,
   sperm cells that have been frozen and thawed for about 12 hours for said collection of sperm cells, and
   sperm cells that have been frozen and thawed overnight for said collection of sperm cells.
180. A system for separation of cells as described in clause 176, or any other clause, wherein said cellular process differential transition effect timer comprises a cellular process differential transition effect timer selected from a group consisting of:
   sperm cells that have been subjected to a differential change inducing buffer for about 30 minutes for said collection of sperm cells,
   sperm cells that have been subjected to a differential change inducing buffer for about 45 minutes for said collection of sperm cells,
   sperm cells that have been subjected to a differential change inducing buffer for about 60 minutes for said collection of sperm cells,
   sperm cells that have been subjected to a differential change inducing buffer for about 90 minutes for said collection of sperm cells, and
   and all permutations and combinations of each of the above.
181. A system for separation of cells as described in clauses 120, 121, 122, 123, 126, or any other clause, wherein said collection of cells initially exist in seminal plasma, and further comprising a seminal plasma-less collection of sperm cells for said initial collection of sperm cells.
182. A system for separation of cells as described in clause 181, or any other clause, and further comprising a seminal plasma included collection of sperm cells for said collection of sperm cells at the time of accomplishing separation.
183. A system for separation of cells as described in clause 181, or any other clause, and further comprising a collection of sperm cells is selected from a group consisting of:
   a seminal plasma included collection of sperm cells for said collection of sperm cells at the time of accomplishing separation, and
   a BSA included collection of sperm cells for said collection of sperm cells at the time of accomplishing separation.
184. A system for separation of cells as described in clause 148, or any other clause, and further comprising an induced sperm cell sex chromosome related differential effect quencher.
185. A system for separation of cells as described in clause 174, or any other clause, and further comprising a sperm cell induced capacitation quencher.
186. A system for separation of cells as described in clause 185, or any other clause, wherein said sperm cell induced capacitation quencher comprises a sperm cell induced capacitation quencher selected from a group consisting of:
   a seminal plasma included collection of sperm cells for said collection of sperm cells at the time of accomplishing separation, and
   a BSA included collection of sperm cells for said collection of sperm cells at the time of accomplishing separation.
187. A system for separation of cells as described in clauses 120, 121, 122, 123, or any other clause, and further comprising a suspension of sperm cell associatable particles in said collection of sperm cells.
188. A system for separation of cells as described in clauses 120, 121, 122, 123, 124, or any other clause, and further comprising an unsuspended collection of sperm cell associatable particles in said collection of sperm cells.
189. A system for separation of cells as described in clauses 120, 121, 122, 123, 124, or any other clause, and further comprising a mixture of sperm cell associatable particles in said collection of sperm cells.
190. A system for separation of cells as described in clauses 120, 121, 122, 123, 124, or any other clause, and further comprising A sperm cell passageway past said sperm cell associatable particles.
191. A system for separation of cells as described in clauses 120, 121, 122, 123, 124, or any other clause, and further comprising A sperm cell associatable particles passageway past said sperm cells.
192. A system for separation of cells as described in clauses 120, 121, 122, 123, 124, or any other clause, and further comprising sperm cell associatable particles having a mean diameter at least about 1000 nm.
193. A system for separation of cells as described in clause 123, or any other clause, and further comprising a sperm cell associatable substance.
194. A system for separation of cells as described in clauses 124, 193 or any other clause, wherein said substance or particles comprise specific chromosome bearing type of sperm cell associatable particles.
195. A system for separation of cells as described in clauses 120, 121, 122, 123, 193, or any other clause, wherein said substance or particles comprise electrostatically sperm cell associatable particles.
196. A system for separation of cells as described in clauses 120, 121, 122, 123, 193, or any other clause, wherein said substance or particles comprise chemically sperm cell associatable particles.
197. A system for separation of cells as described in clauses 195, 196, or any other clause, wherein said particles comprise coated sperm cell associatable particles.
198. A system for separation of cells as described in clause 197, or any other clause, wherein said coated sperm cell associatable particles comprise coated sperm cell associatable particles selected from a group consisting of:
   carboxyl modified silane coated sperm cell associatable particles,
   carbohydrate coated sperm cell associatable particles,
   ligand coated sperm cell associatable particles,
   Sambucus nigra agglutinin (SNA) coated sperm cell associatable particles,
   Monosaccharide coated sperm cell associatable particles,
   antibody coated sperm cell associatable particles,
   sperm cell differentiatable condition active sperm cell associatable particles,
   polymerase associatable particles,
   receptor molecule associatable particles,
   Cas9-type associatable particles,
   CRISPR-type associatable particles,
   DNA tag associatable particles,
   and all permutations and combinations of each of the above.
199. A system for separation of cells as described in clause 123, or any other clause, and further comprising differentially associatable particles.
200. A system for separation of cells as described in clauses 121, 122, 123, or any other clause, and further comprising a fluid combination of sperm cell associatable particles and sperm cells.
201. A system for separation of cells as described in clause 124, 125, 146, or any other clause, and further comprising cell associatable particles selected from a group consisting of:
   carboxyl modified silane coated sperm cell associatable particles,
   carbohydrate coated sperm cell associatable particles,
   ligand coated sperm cell associatable particles,
   Sambucus nigra agglutinin (SNA) coated sperm cell associatable particles,
   Monosaccharide coated sperm cell associatable particles,
   antibody coated sperm cell associatable particles,
   sperm cell differentiatable condition active sperm cell associatable particles,
   polymerase associatable particles,
   receptor molecule associatable particles,
   Cas9-type associatable particles,
   CRISPR-type associatable particles,
   DNA tag associatable particles,
   and all permutations and combinations of each of the above.
202. A system for separation of cells as described in clauses 120, 124, 200, 201, or any other clause, wherein said fluid combination comprises fluid combination of cell associatable nanoparticles and cells.
203. A system for separation of cells as described in clause 120, 124, 200, 201, or any other clause, wherein said fluid combination comprises a fluid combination containing items selected from a group consisting of:
   cell associatable nanoparticles,
   cell associatable particles having a mean diameter of between 10 nm and 999 nm,
   cell associatable microparticles,
   cell associatable particles having a mean diameter of between 100 nm and 100 um,
   cell associatable particles having a mean diameter of not more than about 1000 nm,
   cell associatable particles having a mean diameter of about 670 nm,
   cell associatable particles having a mean diameter at least about 100 nm,
   cell associatable particles having a mean diameter at least about 300 nm,
   cell associatable particles having a mean diameter at least about 500 nm,
   cell associatable particles having a mean diameter at least about 600 nm, and
   all permutations and combinations of each of the above.
204. A system for separation of cells as described in clause 120, 121, 122, 123, 199, 201 or any other clause, wherein said separation modality comprises a separation modality selected from a group consisting of:
   iron oxide particles,
   glass particles,
   silica particles,
   silica with aluminum substitution particles,
   borosilicate particles,
   plastic particles,
   PVP particles,
   polyvinlypropylene particles,
   polyvinylpyrrolidone particles,
   polystyrene particles,
   melamine particles,
   PMMA particles,
   polylactide particles,
   particles bound to polar molecules,
   dextran particles,
   functionalized surface particles, and
   and all permutations and combinations of each of the above.
205. A system for separation of cells as described in clause 201, 202, or any other clause, wherein said fluid combination of cell associatable nanoparticles and cells comprises fluid combination of cell associatable nanoparticles and sperm cells selected from a group consisting of:
   particles stabilized by pH adjustment that establish a fluid combination of cell associatable particles and cells,
   pH particle stabilized particles that establish a fluid combination of cell associatable particles and cells,
   particle concentrated particles fluid that establish a fluid combination of cell associatable particles and cells,
   a stable particle fluid that establishes a fluid combination of cell associatable particles and cells,
   a stable concentration level particle fluid that establishes a fluid combination of cell associatable particles and cells,
   a particle size-concentration level coordinated stable particle fluid that establishes a fluid combination of cell associatable particles and cells,
   a 50 nm particle size-50% wt solids particle fluid that establishes a fluid combination of cell associatable particles and cells,
   a 10 nm particle size-30% wt solids particle fluid that establishes a fluid combination of cell associatable particles and cells, and
   and all permutations and combinations of each of the above.
206. A system for separation of cells as described in clauses 120, 124, 200, 201, or any other clause, wherein said separation modality comprises a magnetic cell separation modality.
207. A system for separation of cells as described in clauses 120, 124, 200, 201, or any other clause, wherein said separation modality comprises an electrostatic cell separation modality
208. A system for separation of cells as described in clauses 120, 124, 200, 201, or any other clause, wherein said separation modality comprises an electrophoretic cell separation modality
209. A system for separation of cells as described in clauses 120, 124, 200, 201, or any other clause, wherein said separation modality comprises a gravimetric cell separation modality.
210. A system for separation of cells as described in clause 209, or any other clause, wherein said separation modality comprises cell associatable microparticles.
211. A system for separation of cells as described in clause 210, or any other clause, wherein said cell associatable particles comprise cell associatable particles having a mean diameter at least about 1000 nm.
212. A system for separation of cells as described in clause 209, or any other clause, wherein said gravimetric cell separation modality comprises a greater than cell motility effect gravimetric cell separation modality.
213. A system for separation of cells as described in clause 209, or any other clause, wherein said gravimetric cell separation modality comprises a centrifugation cell separation modality.
214. A system for separation of cells as described in clauses 120, 121, 112, 124, or any other clause, wherein said separation modality comprises a bulk cell separation modality.
215. A system for separation of cells as described in clauses 123, 125, 146, 214, or any other clause, wherein said bulk separation modality comprises a bulk cell separation modality that acts while said cells are in said collection of cells.
216. A system for separation of cells as described in clauses 123, 125, 146, 214, or any other clause, wherein said bulk separation modality comprises a bulk separation modality selected from a group consisting of:
   a more than one-at-a-time separation modality,
   a simultaneous significant quantity cell separation modality,
   a simultaneous majority of the desired type of cell separation modality,
   a simultaneous substantially all of the desired type of cell separation modality, and
   an at least ten thousand of said cells at a time simultaneous cell separation modality.
217. A system for separation of cells as described in clauses 120, 112, 122, 123, 124, or any other clause, wherein said collection of cells comprises a collection of cells selected from a group consisting of:
   a no unnatural substance for one type of cells collection of cells,
   a no stain collection of cells,
   a no hyperactivity collection of cells,
   a no physical activity collection of cells, and
   and all permutations and combinations of each of the above.
218. A system for separation of cells as described in clauses 120, 112, 122, 123, 124, or any other clause, wherein said separation modality comprises a non-cell motility separation modality.
219. A system for separation of cells as described in clauses 123, 124, or any other clause, wherein said separation modality comprises an external force separation modality.
220. A system for separation of cells as described in clause 219, or any other clause, wherein said external force separation modality comprises an external force separation modality selected from a group consisting of:
   a magnetic cell separation modality,
   an electrostatic cell separation modality,
   an electrophoretic cell separation modality, and
   a gravimetric cell separation modality.
221. A system for separation of cells as described in clauses 120, 112, 122, 123, 124, or any other clause, wherein said separation modality comprises a separation modality selected from a group consisting of:
   an at least about 70% yield of a desired type of cell separation modality,
   an at least about 80% yield of a desired type of cell separation modality,
   an at least about 90% yield of a desired type of cell separation modality,
   an at least about 95% yield of a desired type of cell separation modality,
   an at least about 97% yield of a desired type of cell separation modality,
   an at least about 98% yield of a desired type of cell separation modality, and
   an at least about 99% yield of a desired type of cell separation modality.
222. A system for separation of cells as described in clauses 120, 112, 122, 123, 124, or any other clause, wherein said separation modality comprises a desired type of cell capture element.
223. A system for separation of cells as described in clauses 120, 112, 122, 123, 124, or any other clause, wherein said separation modality comprises a gravimetric cell separation modality.
224. A system for separation of cells as described in clauses 124, 223, or any other clause, wherein said gravimetric cell separation modality comprises a gravitational cell separation modality.
225. A system for separation of cells as described in clauses 124, 223, or any other clause, wherein said gravimetric cell separation modality comprises an enhanced force cell separation modality.
226. A system for separation of cells as described in clauses 124, 223, or any other clause, wherein said gravimetric cell separation modality comprises a centrifugation cell separation modality.
227. A system for separation of cells as described in clause 124, or any other clause, wherein said cells comprise sperm cells.
228. A system for separation of cells as described in clause 227, or any other clause, wherein said sperm cells comprise X-bearing sperm cells and Y-bearing sperm cells.
229. A system for separation of cells as described in clause 228, or any other clause, wherein said X-bearing sperm cells and Y-bearing sperm cells comprise X-bearing sperm cells and Y-bearing sperm cells for artificial insemination use.
230. A system for separation of cells as described in clause 227, or any other clause, wherein said sperm cells comprise dying or functionally impaired sperm cells.
231. A system for separation of cells as described in clause 124, or any other clause, wherein said cells comprise dying or functionally impaired cells.

As can be easily understood from the foregoing, the basic concepts of the present invention may be embodied in a variety of ways. It involves both separation techniques as well as devices to accomplish the appropriate substances and equipment described. In this application, the separation techniques are disclosed as part of the results shown to be achieved by the various steps and utilized devices described and as steps which are inherent to utilization. They are simply the natural result of utilizing the devices as intended and described. In addition, while some devices are disclosed, it should be understood that these not only accomplish certain methods but also can be varied in a number of ways. Importantly, as to all of the foregoing, all of these facets should be understood to be encompassed by this disclosure.

The discussion included in this application is intended to serve as a basic description. The reader should be aware that the specific discussion may not explicitly describe all embodiments possible; many alternatives are implicit. It also may not fully explain the generic nature of the invention and may not explicitly show how each feature or element can actually be representative of a broader function or of a great variety of alternative or equivalent elements. Again, these are implicitly included in this disclosure. Where the invention is described in device-oriented terminology, each element of the device implicitly performs a function. Apparatus claims may not only be included for the device described, but also method or process claims may be included to address the functions the invention and each element performs. Neither the description nor the terminology is intended to limit the scope of the claims that will be included in any subsequent patent application.

It should also be understood that a variety of changes may be made without departing from the essence of the invention. Such changes are also implicitly included in the description. They still fall within the scope of this invention. A broad disclosure encompassing both the explicit embodiment(s) shown, the great variety of implicit alternative embodiments, and the broad methods or processes and the like are encompassed by this disclosure and may be relied upon when drafting the claims for any subsequent patent application. It should be understood that such language changes and broader or more detailed claiming may be accomplished at a later date (such as by any required deadline) or in the event the applicant subsequently seeks a patent filing based on this filing. With this understanding, the reader should be aware that this disclosure is to be understood to support any subsequently filed patent application that may seek examination of as broad a base of claims as deemed within the applicant's right and may be designed to yield a patent covering numerous aspects of the invention both independently and as an overall system. Further, each of the various elements of the invention and claims may also be achieved in a variety of manners. Additionally, when used or implied, an element is to be understood as encompassing individual as well as plural structures that may or may not be physically connected. This disclosure should be understood to encompass each such variation, be it a variation of an embodiment of any apparatus embodiment, a method or process embodiment, or even merely a variation of any element of these. Particularly, it should be understood that as the disclosure relates to elements of the invention, the words for each element may be expressed by equivalent apparatus terms or method terms—even if only the function or result is the same. Such equivalent, broader, or even more generic terms should be considered to be encompassed in the description of each element or action. Such terms can be substituted where desired to make explicit the implicitly broad coverage to which this invention is entitled. As but one example, it should be understood that all actions may be expressed as a means for taking that action or as an element which causes that action. Similarly, each physical element disclosed should be understood to encompass a disclosure of the action which that physical element facilitates. Regarding this last aspect, as but one example, the disclosure of an “extender” should be understood to encompass disclosure of the act of “extending”—whether explicitly discussed or not—and, conversely, were there effectively disclosure of the act of “extending”, such a disclosure should be understood to encompass disclosure of an “extender” and even a “means for extending”. Such changes and alternative terms are to be understood to be explicitly included in the description. Further, each such means (whether explicitly so described or not) should be understood as encompassing all elements that can perform the given function, and all descriptions of elements that perform a described function should be understood as a non-limiting example of means for performing that function.

Any patents, publications, or other references mentioned in this application for patent are hereby incorporated by reference. Any priority case(s) claimed by this application is hereby appended and hereby incorporated by reference. In addition, as to each term used it should be understood that unless its utilization in this application is inconsistent with a broadly supporting interpretation, common dictionary definitions should be understood as incorporated for each term and all definitions, alternative terms, and synonyms such as contained in the Random House Webster's Unabridged Dictionary, second edition are hereby incorporated by reference. Finally, all references listed in the list of References To Be Incorporated By Reference In Accordance With The Provisional Patent Application or other information statement filed with the application are hereby appended and hereby incorporated by reference, however, as to each of the above, to the extent that such information or statements incorporated by reference might be considered inconsistent with the patenting of this/these invention(s) such statements are expressly not to be considered as made by the applicant(s).

REFERENCES TO BE INCORPORATED BY REFERENCE
I. US PATENTS

| U.S. Pat. No. | Kind Code | Date Issued | Patentee |
|---|---|---|---|
| 5,135,759 | | 1992 Aug. 4 | Johnson |
| 9,804,153 | B2 | 2017 Oct. 31 | Krug |

II. US PUBLICATIONS

| Publication No. | Kind Code | Date Published | Patentee |
|---|---|---|---|
| 20140234864 | A1 | 2014 Aug. 21 | Krug |
| 20160091410 | A1 | 2016 Mar. 31 | Krug |
| 20120270204 | A1 | 2012 Oct. 25 | Fox et al. |
| 20100081130 | A1 | 2010 Apr. 1 | Lee et al. |
| 20120100546 | A1 | 2012 Apr. 26 | Lowery, J R. et al. |
| 20020034537 | A1 | 2002 Mar. 21 | Schulze et al. |

III. FOREIGN PATENTS

| U.S. Pat. No. | Kind Code | Country Code | Date Issued | Patentee |
|---|---|---|---|---|
| 2014035840 | A1 | WO | 2014 Mar. 6 | Krug |
| 2016090310 | A1 | WO | 2016 Jun. 9 | Krug |
| 2,890,498 | A1 | EP | 2015 Jul. 8 | Krug |
| 0,113,452 | A2 | EP | 1984 Jul. 18 | Benner |

IV. NON-PATENT LITERATURE
U.S. Provisional Application No. 62,584,598, First Named Inventor Krug
WOLF, C. A., The effect of sperm selection by Percoll or swim-up on the sex ratio of in vitro
produced bovine embryos, Anim. Reprod., v.5, n.3/4, p. 110-115, July/December 2008, 6 pages
SCIELO, Sex selection in bovine spermatozoa by using Percoll discontinues density gradient
centrifugation, http://www.scielo.br/scielo, Oct. 17, 2018. 6 pages
PARRISH, et al., Capacitation of Bovine Sperm by Heparin, Biology of Reproduction 38,
1171-1180 (1988), 10 pages
CARTWRIGHT, et al., Separation of bovine X and Y sperm based on surface differences, The
University of Manchester, www.research.manchester.ac.uk/portal/en/publications, Oct. 18,
2018, 3 pages
CHAN, et al., A simple zeta method for sperm selection based on membrane charge, Loma
Linda University School of Medicine, Departments of Gynecology and Obstetrics and
Physiology and Pharmacology, Center for Fertility and In Vitro Fertilization, Techniques and
Instrumentation, Vol. 85, No. 2, February 2006, 6 pages
DOMINGUEZ, et al., Sperm Sexing Mediated by Magnetic Nanoparticles in Donkeys, a
Preliminary In Vitro Study, Journal of Equine Veterinary Science 65 (2018) 123-127, 4 pages
ENGELMANN, et al, Separation of human X and Y spermatozoa by free-flow
electrophoresis, Wiley Online Library, Gamete Research/Volume 19, Issue 2, First published:
February 1988; https://doi.org/10.1002/mrd.1120190205 Cited by: 24, Oct. 18, 2018, 3
pages
KOOIJ, et al., Determination of sex ratio of spermatozoa with a deoxyribonucleic acid-probe
and quinacrine staining: a comparison, Urology-andrology Fertility and Sterility, Vol. 58, No.
2, August 1992, 3 pages
IQBAL, et al., Comparison of Various Bovine Sperm Capacitation Systems for Their Ability
to Alter the Net Negative Surface Charge of Spermatozoa, University of Minnesota,
Department of Animal Science, 1995 J Dairy Sci 78: 84-90, 7 pages
KANEKO, et al., Human X- and Y-Bearing Sperm Differ in Cell Surface Sialic Acid Content,
Biochemical and Biophysical Research Communications Vol. 124, Pages 950-955, No. 3,
Nov. 14, 1984, 6 pages
NEVO, et al., “Electrophoretic properties of bull and of rabbit spermatozoa,” Experimental
Cell Research, 23: 69-83 (1961)
IQBAL et al., “Comparison of Various Bovine Sperm Capacitation Systems for Their Ability
to Alter the Net Negative Surface Charge of Sperm,” J Dairy Sci 78: 84-90 (1995)
HAMMERSTEDT et al., “Use of amphiphilic spin labels and whole cell isoelectric focusing
to assay charge characteristics of sperm surfaces,” Arch. Biochem. Biophys., 194: 565-580
(1979)
FAROOQUI, “Biochemistry of sperm capacitation,” Int. J. Biochem., 15: 463-468 (1983)
YANAGIMACHI, R and Usui, “Calcium dependence of the acrosome reaction and activation
of guinea pig spermatozoa,” Experimental Cell Research, 89: 161-174 (1974)
FOCARELLI et al., “Sialylglycoconjugates release during in vitro capacitation of human
spermatozoa. J. Andrology. Vol. 11, No. 2: 97-104 (1990)
MURPHY AND YANAGIMACHI, “The pH dependence of motility and acrosome reaction of
guinea pig spermatozoa,” Gamete Res. 10: 1 (1984)
SCHENK J L AND GE SEIDEL JR. Cryopreservation of Flow-Sorted Bovine Spermatozoa.
Theriogenology 52: 1375-1391 (1999)
IQBAL I AND HUNTER G, “Comparison of Various Bovine Sperm Capacitation Systems for
Their Ability to Alter the Net Negative Surface Charge of Spermatozoa, J. Dairy Science
78: 84-90 (1995)
ROGERS, “Mammalian Sperm Capacitation and Fertilization In vitro: A Critique of
Methodology,” Gamete Research, 1: 165-223 (1978)
PARRISH, et al., Effect of Bovine Sperm Separation by Either Swim-Up or Percoll Method
on Success of In Vitro Fertilization and Early Embryonic Development, University of
Wisconsin, Department of Meat and Animal Science, received for publication: Sep. 8,
1994, Accepted: Mar. 3, 1995, 11 pages
MACHADO, et al., Effect of Percoll volume, duration and force of centrifugation, on in vitro
production and sex ratio of bovine embryos, ScienceDirect, Theriogenology 71 (2009) 1289-
1297, Received 13 Sep. 2008; received in revised form 11 Dec. 2008; accepted 8
January 2009, 9 pages
SCHENK, et al., Cryopreservation of Flow-Sorted Bovine Spermatozoa, Elsevier, received for
publication: Aug. 26, 1999, Accepted: Oct. 12, 1999, 17 pages
HAQUE, et al., Sperm Sexing and its Application in Livestock Sector, International Journal of
Current Microbiology and Applied Sciences ISSN: 2319-7692 Special Issue-7 pp. 259-272;
Journal homepage: http://www.ijcmas.com, Int. J. Curr. Microbiol. App. Sci (2018) Special Issue-
7: 259-272, January 2018, 15 pages
VREDENBURGH-WILBERG, et al., Intracellular pH of Bovine Sperm Increases During
Capacitation, University of Wisconsin, Department of Meat and Animal Science, Molecular
Reproduction and Development 40: 490-502 (1995), Received Jul. 6, 1994; accepted
Sep. 16, 1994, 13 pages
JOHNSON AND HUNTER, “Seminal Antigens: Their Alteration in the Genital Tract of
Female Rabbits and during Partial In Vitro capacitation with Beta Amylase and Beta
Glucuronidase,” Biology of Reproduction, 7: 332-340, (1972)
LANGLAIS AND ROBERTS, “A molecular membrane model of sperm capacitation and the
acrosome reaction of mammalian spermatozoa. Gamete Research. 12: 183-224 (1985)
U.S. patent application No. 13,974,139, First Named Inventor Krug
U.S. patent application No. 14,960,096, First Named Inventor Krug
U.S. patent application No. 15,713,391, First Named Inventor Krug
U.S. Provisional Pat. application No. 61,694,756, First Named Inventor Krug
U.S. Provisional Pat. application No. 62,088,425, First Named Inventor Krug
International Patent Application No. PCT/US2013/056526, Search Report and Written
Opinion, mailed Feb. 7, 2014, 47 pages.
International Patent Application No. PCT/US2015/064098, Search Report and Written
Opinion, mailed Feb. 12, 2016, 8 pages.

Thus, the applicant(s) should be understood to have support to claim and make a statement of invention to at least: i) each of the utilized devices as herein disclosed and described, ii) the related methods disclosed and described, iii) similar, equivalent, and even implicit variations of each of these devices and methods, iv) those alternative designs which accomplish each of the functions shown as are disclosed and described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products, substances, mixes, and animals produced by such process, methods, systems or components, ix) each system, method, and element shown or described as now applied to any specific field or devices mentioned, x) methods and apparatuses substantially as described hereinbefore and with reference to any of the accompanying examples, xi) an apparatus for performing the methods described herein comprising means for performing the steps, xii) the various combinations and permutations of each of the elements disclosed, xiii) each potentially dependent claim or concept as a dependency on each and every one of the independent claims or concepts presented, and xiv) all inventions described herein.

With regard to claims whether now or later presented for examination, it should be understood that for practical reasons and so as to avoid great expansion of the examination burden, the applicant may at any time present only initial claims or perhaps only initial claims with only initial dependencies. The office and any third persons interested in potential scope of this or subsequent applications should understand that broader claims may be presented at a later date in this case, in a case claiming the benefit of this case, or in any continuation in spite of any preliminary amendments, other amendments, claim language, or arguments presented, thus throughout the pendency of any case there is no intention to disclaim or surrender any potential subject matter. It should be understood that if or when broader claims are presented, such may require that any relevant prior art that may have been considered at any prior time may need to be re-visited since it is possible that to the extent any amendments, claim language, or arguments presented in this or any subsequent application are considered as made to avoid such prior art, such reasons may be eliminated by later presented claims or the like. Both the examiner and any person otherwise interested in existing or later potential coverage, or considering if there has at any time been any possibility of an indication of disclaimer or surrender of potential coverage, should be aware that no such surrender or disclaimer is ever intended or ever exists in this or any subsequent application. Limitations such as arose in Hakim v. Cannon Avent Group, PLC, 479 F.3d 1313 (Fed. Cir 2007), or the like are expressly not intended in this or any subsequent related matter. In addition, support should be understood to exist to the degree required under new matter laws—including but not limited to European Patent Convention Article 123(2) and United States Patent Law 35 USC 132 or other such laws—to permit the addition of any of the various dependencies or other elements presented under one independent claim or concept as dependencies or elements under any other independent claim or concept. In drafting any claims at any time whether in this application or in any subsequent application, it should also be understood that the applicant has intended to capture as full and broad a scope of coverage as legally available. To the extent that insubstantial substitutes are made, to the extent that the applicant did not in fact draft any claim so as to literally encompass any particular embodiment, and to the extent otherwise applicable, the applicant should not be understood to have in any way intended to or actually relinquished such coverage as the applicant simply may not have been able to anticipate all eventualities; one skilled in the art, should not be reasonably expected to have drafted a claim that would have literally encompassed such alternative embodiments.

Further, if or when used, the use of the transitional phrase “comprising” is used to maintain the “open-end” claims herein, according to traditional claim interpretation. Thus, unless the context requires otherwise, it should be understood that the term “comprise” or variations such as “comprises” or “comprising”, are intended to imply the inclusion of a stated element or step or group of elements or steps but not the exclusion of any other element or step or group of elements or steps. Such terms should be interpreted in their most expansive form so as to afford the applicant the broadest coverage legally permissible. The use of the phrase, “or any other claim” is used to provide support for any claim to be dependent on any other claim, such as another dependent claim, another independent claim, a previously listed claim, a subsequently listed claim, and the like. As one clarifying example, if a claim were dependent “on claim 20 or any other claim” or the like, it could be re-drafted as dependent on claim 1, claim 15, or even claim 25 (if such were to exist) if desired and still fall with the disclosure. It should be understood that this phrase also provides support for any combination of elements in the claims and even incorporates any desired proper antecedent basis for certain claim combinations such as with combinations of method, apparatus, process, and the like claims.

Finally, any claims set forth at any time are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

What is claimed is:

1. A method for separation of X-bearing sperm cells and Y-bearing sperm cells comprising the steps of:
establishing a collection of sperm cells that has both X-bearing sperm cells and Y-bearing sperm cells in said collection;
inducing a sperm cell sex chromosome related differential effect in said collection of sperm cells;
combining sperm cell associatable particles with said collection of sperm cells to establish a fluid combination of sperm cell associatable particles and sperm cells;
associating a desired portion of said collection of sperm cells with at least some of said sperm cell associatable particles in said fluid combination of sperm cell associatable particles and sperm cells;
separating at least some of said X-bearing sperm cells and Y-bearing sperm cells through action of said sperm cell associatable particles in said fluid combination of sperm cell associatable particles and sperm cells.

2. A method of separating cells as described in claim 1 wherein both said X-bearing and Y-bearing sperm cells in said collection prior to separation comprise functionally viable X-bearing and Y-bearing sperm cells.

3. A method of separating cells as described in claim 1 wherein said functionally viable cells usable in practical application for fertilization processes comprise functionally viable cells usable in practical application for artificial insemination processes.

4. A method of separating cells as described in claim 1, wherein both said X-bearing and Y-bearing sperm cells in said collection prior to separation comprise sperm cells in a state where they are practically usable for fertilization without substantial loss of such cells selected from a group consisting of:
after overnight storage,
after shipping in a natural state,
after freezing, shipping and thawing,
after having been frozen,
after having been frozen and then thawed,
after at least about 8 hours for cells held in an unfrozen state in seminal plasma,
after at least about 16 hours for cells held in an unfrozen state in seminal plasma,
after at least about 24 hours for cells held in an unfrozen state in seminal plasma,
after at least about 30 minutes after thawing for cells frozen and then thawed,
after at least about 45 minutes after thawing for cells frozen and then thawed,
after at least about 1 hour after thawing for cells frozen and then thawed,
after at least about 2 hours after thawing for cells frozen and then thawed,
cells that remain practically usable for fertilization without a loss of more than 20% of such cells,
cells that remain practically usable for fertilization without a loss of more than 30% of such cells,
cells that remain practically usable for fertilization without a loss of more than 40% of such cells,
and all permutations and combinations of each of the above.

5. A method of separating cells as described in claim 1 wherein said functionally viable X-bearing and Y-bearing sperm cells comprise capacitation triggered sperm cells.

6. A method of separating cells as described in claim 1, wherein said capacitation triggered sperm cells comprise pre-acrosome reaction initiation sperm cells.

7. A method of separating cells as described in claim 1, and further comprising the step of selecting sperm cells that have never been frozen for said collection of sperm cells.

8. A method of separating cells as described in claim 1, and further comprising the step of selecting sperm cells that have been frozen and then thawed for said collection of sperm cells.

9. A method of separating cells as described in clauses 1, and further comprising the step of selecting human sperm cells for said collection of sperm cells.

10. A method of separating cells as described in clause 1 and further comprising the step of selecting bovine sperm cells for said collection of sperm cells.

11. A method of separating cells as described in clauses 1, and further comprising the step of selecting porcine sperm cells for said collection of sperm cells.

12. A method of separating cells as described in claim 1, wherein said step of inducing a sperm cell sex chromosome related differential effect comprises the step of controlled difference inducing a sperm cell sex chromosome related differential effect.

13. A method of separating cells as described in claim 1, and further comprising the step of subjecting said collection of sperm cells to heparin.

14. A method of separating cells as described in claim 1, wherein said step of subjecting said collection of sperm cells to heparin comprises the step of subjecting said collection of sperm cells to heparin under conditions selected from a group consisting of:
a concentration of about 5 ug heparin per ml of buffer,
a concentration of about 10 ug heparin per ml of buffer,
a concentration of about 15 ug heparin per ml of buffer,
a concentration of about 20 ug heparin per ml of buffer,
a concentration of about 5 ug heparin per ml of buffer for about 120 minutes for sperm cells that have not been previously frozen,
a concentration of about 5 ug heparin per ml of buffer for about 180 minutes for sperm cells that have not been previously frozen,
a concentration of about 5 ug heparin per ml of buffer for from about 180 minutes to about 240 minutes for sperm cells that have not been previously frozen,
a concentration of about 10 ug heparin per ml of buffer for about 120 minutes for sperm cells that have not been previously frozen,
a concentration of about 10 ug heparin per ml of buffer for about 180 minutes for sperm cells that have not been previously frozen,
a concentration of about 10 ug heparin per ml of buffer for from about 180 minutes to about 240 minutes for sperm cells that have not been previously frozen,
a concentration of about 15 ug heparin per ml of buffer for about 120 minutes for sperm cells that have not been previously frozen,
a concentration of about 15 ug heparin per ml of buffer for about 180 minutes for sperm cells that have not been previously frozen,
a concentration of about 15 ug heparin per ml of buffer for from about 180 minutes to about 240 minutes for sperm cells that have not been previously frozen,
a concentration of about 20 ug heparin per ml of buffer for about 120 minutes for sperm cells that have not been previously frozen,
a concentration of about 20 ug heparin per ml of buffer for about 180 minutes for sperm cells that have not been previously frozen,
a concentration of about 20 ug heparin per ml of buffer for from about 180 minutes to about 240 minutes for sperm cells that have not been previously frozen,
a concentration of about 5 ug heparin per ml of buffer for about 30 minutes for thawed previously frozen sperm cells,
a concentration of about 5 ug heparin per ml of buffer for about 60 minutes for thawed previously frozen sperm cells,
a concentration of about 5 ug heparin per ml of buffer for from about 45 minutes to about 60 minutes for thawed previously frozen sperm cells,
a concentration of about 10 ug heparin per ml of buffer for about 30 minutes for thawed previously frozen sperm cells,
a concentration of about 10 ug heparin per ml of buffer for about 60 minutes for thawed previously frozen sperm cells,
a concentration of about 10 ug heparin per ml of buffer for from about 45 minutes to about 60 minutes for thawed previously frozen sperm cells,
a concentration of about 15 ug heparin per ml of buffer for about 30 minutes for thawed previously frozen sperm cells,
a concentration of about 15 ug heparin per ml of buffer for about 60 minutes for thawed previously frozen sperm cells,
a concentration of about 15 ug heparin per ml of buffer for from about 30 minutes to about 60 minutes for thawed previously frozen sperm cells,
a concentration of about 20 ug heparin per ml of buffer for about 30 minutes for thawed previously frozen sperm cells,
a concentration of about 20 ug heparin per ml of buffer for about 60 minutes for thawed previously frozen sperm cells,
a concentration of about 20 ug heparin per ml of buffer for from about 30 minutes to about 60 minutes for thawed previously frozen sperm cells,
until exhibiting an increase of about 0.33 pH,
until exhibiting an increase of about 0.36 pH,
until exhibiting an increase of about 0.39 pH,
until exhibiting an optimal differential effect increase in pH,
until exhibiting an optimal cell viability increase in pH,
and all permutations and combinations of each of the above.

15. A method of separating cells as described in claim 1, and further comprising the steps of:
determining a usable level of sperm cell sex chromosome related differential effect, and
affirmatively effecting said level of sperm cell sex chromosome related differential effect for said collection of sperm cells.

16. A method of separating cells as described in claim 1, wherein said step of determining a usable pH indicated level of sperm cell sex chromosome related differential effect comprises the step of determining a usable pH indicated level of sperm cell sex chromosome related differential effect selected from a group consisting of:
determining a pH increase for the environment of said collection of sperm cells of about 0.33 pH,
determining a pH increase for the environment of said collection of sperm cells of about 0.36 pH, and
determining a pH increase for the environment of said collection of sperm cells of about 0.39 pH.

17. A method of separating cells as described in claim 1, wherein said step of determining a usable level of sperm cell sex chromosome related differential effect comprises the step of timing a cellular process differential transition effect for said collection of sperm cells.

18. A method of separating cells as described in claim 1, wherein said step of timing a cellular process differential transition effect for said collection of sperm cells comprises the step of timing a cellular process differential transition effect for said collections of sperm cells selected from a group consisting of:
utilizing frozen-thawed sperm cells after having been thawed for about 4 hours,
utilizing frozen-thawed sperm cells after having been thawed for about 6 hours,
utilizing frozen-thawed sperm cells after having been thawed for about 8 hours,
utilizing frozen-thawed sperm cells after having been thawed for about 12 hours, and
utilizing frozen-thawed sperm cells after having been thawed for overnight.

19. A method of separating cells as described in claim 1, and further comprising the step of incubating said collection of sperm cells with associatable particles.

20. A method of separating cells as described in claim 1, and further comprising the step of resubjecting said collection of sperm cells to a mixture containing substances selected from a group consisting of:
seminal plasma, and
BSA.

21. A method of separating cells as described in claim 1, and further comprising the step of suspending sperm cell associatable particles with said collection of sperm cells to create a suspension of sperm cell associatable particles in said collection of sperm cells.

22. A method of separating cells as described in claim 1, wherein said step of associating comprises the step of electrostatically associating a desired portion of said collection of sperm cells with said sperm cell associatable particles.

23. A method of separating cells as described in claim 1, wherein said coated sperm cell associatable particles comprise coated sperm cell associatable particles selected from a group consisting of:
carboxyl modified silane coated sperm cell associatable particles,
carbohydrate coated sperm cell associatable particles,
ligand coated sperm cell associatable particles,
Sambucus nigra agglutinin (SNA) coated sperm cell associatable particles,
Monosaccharide coated sperm cell associatable particles,
antibody coated sperm cell associatable particles,
sperm cell differentiatable condition active sperm cell associatable particles,
and all permutations and combinations of each of the above.

24. A method of separating cells as described in claim 1, wherein said step of separating through action of said sperm cell associatable particles comprises the step of separating through action of said sperm cell associatable particles selected from a group consisting of:
iron oxide particles,
glass particles,
silica particles,
silica with aluminum substitution particles,
borosilicate particles,
plastic particles,
PVP particles,
polyvinlypropylene particles,
polyvinylpyrrolidone particles,
polystyrene particles,
melamine particles,
PMMA particles,
polylactide particles,
particles bound to polar molecules,
dextran particles,
functionalized surface particles,
and all permutations and combinations of each of the above.

25. A method of separating cells as described in claim 1, wherein said step of separating comprises the step of magnetically separating at least some of said X-bearing sperm and Y-bearing sperm cells.

26. A method of separating cells as described in claim 1, wherein said step of separating comprises the step of bulk separating said cells.

27. A method of separating cells as described in claim 1, wherein said step of bulk separating comprises the step of bulk separating selected from a group consisting of:
separating said cells more than one-at-a-time,
simultaneously separating a significant quantity of said cells in said collection of cells,
simultaneously separating the majority of the desired type of said cells in said collection of cells,
simultaneously separating substantially all of the desired type of said cells in said collection of cells, and
simultaneously separating at least ten thousand of said cells at a time.

28. A method of separating cells as described in claim 1, wherein said step of selectively impacting comprises the step of causing no substantial effect on one type of cells from said collection of cells.

29. A method of separating cells as described in claim 1, and further comprising the step of separating at least some of one type of cells from said collection of cells while acting substantially passively with respect to another type of said cells.

30. A method of separating cells as described in claim 1, wherein said step of separating at least some of one type of cells from said collection of cells while acting substantially passively with respect to another type of said cells comprises the step of non-cell motility separating at least some of one type of cells from said collection of cells.

31. A method of separating cells as described in claim 1, wherein said step of separating comprises the step of separating selected from a group consisting of:
separating substantially all of a desired type of said cells in said collection of cells,
separating at least about 70% of a desired type of said cells in said collection of cells,
separating at least about 80% of a desired type of said cells in said collection of cells,
separating at least about 90% of a desired type of said cells in said collection of cells,
separating at least about 95% of a desired type of said cells in said collection of cells,
separating at least about 97% of a desired type of said cells in said collection of cells,
separating at least about 98% of a desired type of said cells in said collection of cells,
separating at least about 99% of a desired type of said cells in said collection of cells, and
separating so as to leave no appreciable viable cells of a desired type of said cells in said collection of cells.

32. A method for separation of X-bearing sperm cells and Y-bearing sperm cells comprising the steps of:
establishing a collection of sperm cells that has both X-bearing sperm cells and Y-bearing sperm cells in said collection;
selectively impacting substantially only one type of sex chromosome bearing sperm cells from said collection of sperm cells while leaving the other type of sex chromosome bearing sperm cells from said collection of sperm cells substantially unimpacted;
acting on at least a portion of said only one type of sex chromosome bearing sperm cells that have been selectively impacted; and
separating at least some of said X-bearing sperm cells and Y-bearing sperm cells.

33. A method of separating cells as described in claim 32, wherein both said X-bearing and Y-bearing sperm cells in said collection prior to separation comprise functionally viable X-bearing and Y-bearing sperm cells.

34. A method of separating cells as described in claim 32, wherein said functionally viable cells usable in practical application for fertilization processes comprise functionally viable cells usable in practical application for artificial insemination processes.

35. A method of separating cells as described in claim 32, and further comprising the step of inducing a sperm cell sex chromosome related differential effect.

36. A method of separating cells as described in claim 32, and further comprising the step of separating through action of sperm cell associatable particles.

37. A method of separating cells as described in claim 5, wherein said step of separating comprises the step of bulk separating said cells.

38. A method of separating cells as described in claim 32, wherein said step of bulk separating comprises the step of bulk separating cells while still in said collection of cells.

39. A method of separating cells as described in claim 32, wherein said step of separating comprises the step of separating selected from a group consisting of:
separating substantially all of a desired type of said cells in said collection of cells,
separating at least about 70% of a desired type of said cells in said collection of cells,
separating at least about 80% of a desired type of said cells in said collection of cells,
separating at least about 90% of a desired type of said cells in said collection of cells,
separating at least about 95% of a desired type of said cells in said collection of cells,
separating at least about 97% of a desired type of said cells in said collection of cells,
separating at least about 98% of a desired type of said cells in said collection of cells,
separating at least about 99% of a desired type of said cells in said collection of cells, and
separating so as to leave no appreciable viable cells of a desired type of said cells in said collection of cells.

40. A method for bulk separation of X-bearing sperm cells and Y-bearing sperm cells comprising the steps of:
establishing a collection of sperm cells that has both X-bearing sperm cells and Y-bearing sperm cells in said collection,
acting on at least some of said collection of sperm cells based on said sex differential property of said sperm cells, and
bulk separating at least some of said X-bearing sperm cells and Y-bearing sperm cells based upon said sperm cell sex chromosome related differential effect.

41. A method of separating cells as described in claim 40, wherein both said X-bearing and Y-bearing sperm cells in said collection prior to separation comprise functionally viable X-bearing and Y-bearing sperm cells.

42. A method of separating cells as described in claim 40, wherein said functionally viable cells usable in practical application for fertilization processes comprise functionally viable cells usable in practical application for artificial insemination processes.

43. A method of separating cells as described in claim 40, wherein said capacitation triggered sperm cells comprise pre-acrosome reaction initiation sperm cells.

44. A method of separating cells as described in claim 40, and further comprising the step of affirmatively supporting an intrinsically actionable sperm cell sex chromosome related differential effect for said collection of sperm cells.

45. A method of separating cells as described in claim 40, and further comprising the step of inducing a sperm cell sex chromosome related differential effect.

46. A method of separating cells as described in claim 40, wherein said step of inducing a sperm cell sex chromosome related differential effect comprises the step of controlled difference inducing a sperm cell sex chromosome related differential effect.

47. A method of separating cells as described in claim 40, wherein said step of controlled difference inducing a sperm cell sex chromosome related differential effect comprises the step of triggering capacitation for said collection of sperm cells.

48. A method for bulk separation of cells comprising the steps of:
establishing a collection of cells;
inducing a differential effect for said establishing a collection of cells to establish a collection of differentially exhibiting cells;
acting on at least some of said collection of differentially exhibiting cells based on a differential property of said cells; and
bulk separating at least some of said cells based upon said differential property.
